(19) United States
(12) Patent Application Publication
(10) Pub. No.: US 2024/0183842 A1
(43) Pub. Date: Jun. 6, 2024

(54) Supercharged Biovesicles and Methods of Use Thereof

(71) Applicant: The General Hospital Corporation, Boston, MA (US)

(72) Inventors: Koen Breyne, Reading, MA (US); Stefano Ughetto, Cambridge, MA (US); Xandra O. Breakefield, Newton Center, MA (US)

(21) Appl. No.: 18/287,861

(22) PCT Filed: Apr. 22, 2022
(86) PCT No.: PCT/US2022/071873

§ 371 Date: Oct. 20, 2023

Related U.S. Application Data
(60) Provisional application No. 63/180,489, filed on Apr. 27, 2021.
(60) Provisional application No. 63/178,444, filed on Apr. 22, 2021.

(51) Int. Cl. G01N 33/50; C12N 15/86; C12N 15/88

(52) U.S. Cl. CPC G01N 33/5023; C12N 15/86; C12N 15/88; G01N 33/5076; C12N 2740/15022; C12N 2740/15043

(57) ABSTRACT

Provided herein are compositions comprising positively supercharged proteins (+scProteins), membrane-bound vesicles (EV, VLP, and/or LVV membranes), and cargo, and methods of use thereof to deliver the cargo to the intracellular space of target cells. Also provided are reporter constructs that can be used to detect genome editing events, and methods of use thereof.

### CLAIM OF PRIORITY

This application claims the benefit of U.S. Provisional Application Ser. Nos. 63/178,444, filed on Apr. 22, 2021, and 63/180,489, filed on Apr. 27, 2021. The entire contents of the foregoing are incorporated herein by reference.

### FEDERALLY SPONSORED RESEARCH OR DEVELOPMENT

This invention was made with Government support under Grant Nos. CA232103 and CA069246 awarded by the National Institutes of Health. The Government has certain rights in the invention.

### TECHNICAL FIELD

Provided herein are compositions comprising positively supercharged proteins (+scProteins), cell membrane-derived vesicles (extracellular vesicles (EV), virus-like particles (VLP)s, and lentiviral vectors (LVV)), and cargo, and methods of use thereof to deliver the cargo to the intracellular space of target cells. Also provided are reporter constructs that can be used to detect genome editing events, and methods of use thereof.

### BACKGROUND

Delivery of biomolecules and other cargo to intracellular spaces has been a challenge. Cell membrane-based biovesicles (BVs) are important candidate drug delivery vehicles and comprise extracellular vesicles, virus-like particles, and lentiviral vectors. Also described are compositions and methods for detecting CRISPR-based nucleic acid modifications.

### SUMMARY

Provided herein are compositions comprising biovesicles, wherein the biovesicles comprise a lipid bilayer surrounding an aqueous lumen; a positively supercharged protein; and one or more cargo molecules complexed with the positively supercharged protein. In some embodiments, the biovesicles are extracellular vesicles (EVs), lentiviral vectors (LVVs), or virus-like particles (VLPs).

In some embodiments, the EVs are obtained from a biofluid or tissue obtained from a living animal, preferably a mammal, more preferably a human.

In some embodiments, the EVs are obtained from media in a cell culture (i.e., a cell culture of donor cells that “donate” the EVs). In some embodiments, the positively supercharged protein comprises supercharged eGFP or a variant thereof, preferably +scmCerulean3 or +scGFP. In some embodiments, the +scmCerulean3 is at least 95% identical to SEQ ID NO:4, and has a predicted surface charge of at least +11. In some embodiments, the +scGFP is at least 95% identical to SEQ ID NO:4, and has a predicted surface charge of at least +11.

In some embodiments, the cargo comprises a nucleic acid, a protein, a Ribonucleoprotein (RNP), a combination of DNA and protein, or a small molecules. In some embodiments, the nucleic acid is DNA or RNA. In some embodiments, the cargo is a protein. In some embodiments, the protein is a genome editing or epigenome modulating protein. In some embodiments, the genome editing or epigenome modulating protein is or comprises a CRISPR based nuclease, zinc finger (ZF) or a TALE, optionally fused to an epigenome modulator.

In some embodiments, the positively supercharged protein and the protein cargo are in a fusion protein, optionally wherein the positively supercharged protein is fused to the N or C terminus of the cargo, optionally with a polypeptide linker therebetween.

In some embodiments, the protein is or comprises a CRISPR based nuclease, and the cargo further comprises at least one guide RNA complexed with the CRISPR based nuclease.

Also provided herein are methods for delivering a cargo to a cell or tissues, the method comprising contacting the cell or tissue with a composition as described herein. The cell or tissue can be in vivo (in a living animal), or can be ex vivo or in vitro.

Additionally, provided herein are reporter constructs that comprise a transmembrane domain protein; at least one reporter gene fused to an intracellular portion of the transmembrane domain protein; an affinity tag fused to an extracellular portion of the transmembrane domain protein; optionally at least one reporter gene fused to an extracellular portion of the transmembrane domain protein; and a nucleotide sequence complementary to a target gRNA sequence, preferably comprising a variation, e.g., a pathological variation such as a frame shift or a premature stop codon, wherein the sequence prevents expression of the reporter construct.

In some embodiments, the transmembrane domain protein comprises at least two transmembrane domains and at least one extracellular loop, and wherein the affinity tag is disposed in the at least one extracellular loop. In some embodiments, the transmembrane domain is CD63 tetraspanin.

In some embodiments, the reporter genes comprise one or both of a fluorescent tag and a bioluminescent tag; optionally the fluorescent tag is intracellular and the bioluminescent tag is extracellular.

For example, in some embodiments two gRNAs can be used to cut out a sequence from the extracellular loop. That sequence can have a stop codon or a poly A sequence in-between the 2 gRNAs targets and interrupts construct translation. In that case you don't need a frame shift or stop codon, all guide RNA sequences would work and only when the two gRNAs cut simultaneously would the construct be expressed.

Also provided are methods for detecting CRISPR editing of a target sequence, e.g., a target sequence comprising a variation such as a premature stop codon, in a genome of a cell, the method comprising: expressing in the cell with a reporter construct as described herein; isolating extracellular vesicles EVs from the cell; and detecting expression of the reporter construct by detecting a signal from the reporter gene. In some embodiments, the method further comprises isolating the EVs or cells expressing the construct by contacting the cells or EVs with an affinity-tag binding reagent, and isolating cells or EVs that comprise the affinity tag.

The term “about” is used herein to mean ±10%. In some embodiments, about means ±5%. When about is present before a series of numbers or a range, it is understood that “about” can modify each of the numbers in the series or range. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-N. The number of Lys/Arg surface residues are key for a protein's potential to associate with and enter extracellular vesicles.
(A) Protein surface mutagenesis. Proteins can be genetically reengineered to display different numbers of lysine (Lys) or arginine (Arg) residues on the solvent exposed surface. Green fluorescent protein (GFP) with reduced or increased levels of Lys/Arg residues on the surface are called negative supercharged GFP (−scGFP) or positive supercharged GFP (+scGFP), respectively.
(B) Arginine/Lysine content in supercharged proteins. Number of Lys and Arg incorporated in the supercharged GFP proteins are indicated, respectively.
(C) Recombinant supercharged protein pull-down. Ni-NTA resin pull-down of free recombinant +GFP, GFP or, −GFP to deplete supernatant of GFP fluorescence.
(D) In extracellular vesicle (EV)-free solutions, GFP fluorescence was compared between supernatants (S) after incubation with recombinant −scGFP (n=3), GFP (n=4), or +scGFP (n=4) proteins and the Ni-NTA resin pellet (R). Data are represented in percentage whereby the total fluorescence of the supernatant (S) and Ni-NTA resin pellet (R) represents 100 percent.
(E) +GFP within EVs is not pulled down by Ni-NTA resin. Interaction of recombinant GFP proteins with EVs was investigated through comparing fluorescent levels of EV-associated vs -unassociated free recombinant protein post-GFP affinity pull-down with Ni-NTA resin.
(F) +scProtein generates supercharged EVs (scEVs). Post-incubation of HEK293T EVs with −scGFP (n=3), GFP (n=4) or +scGFP (n=4), we added Ni-NTA resin to evaluate whether GFP loaded HEK293T EVs would remain in the supernatant as scEVs. GFP fluorescence was quantified in both supernatant (S) and resin (R). Data are represented in percentage whereby the total fluorescence of the supernatant (S) and Ni-NTA resin pellet (R) represents 100 percent.
(G) Size exclusion chromatography (SEC) profile of fluorescent recombinant +scGFP. Profile when resolving recombinant +scGFP (n=6) with SEC. A fluorescent signal was detected in the late protein fractions (F) i.e., F14 to F28. Graph represents GFP fluorescence in each SEC fraction. Graph represents GFP fluorescence in each SEC fraction.
(H) SEC profile of non-fluorescent SEC-purified EVs. Non-fluorescent HEK293T EVs were not detectable (n=15). Graph represents GFP fluorescence in each SEC fraction. A transmission electron microscopic image of a non-fluorescent EV used for +scProtein loading is shown. Scalebar=100 nm.
(I) SEC profile of scEVs post-Ni-NTA cleanup. HEK293T scEVs (n=6) were generated through combining +scGFP, non-fluorescent HEK293T EVs and Ni-NTA cleanup. scEVs had a distinct SEC profile whereby GFP fluorescence was observed in the lower SEC fractions, i.e. F7-F12, showing incorporation into EVs.
(J) Correlation of +scProtein content and EV number post-scEV purification in scEV solution. +scProtein loading into EVs was dependent on the number of SEC-purified EVs in solution. Red line demonstrates the non-linear correlation (R2=0.9934) between +scProtein fluorescence and the number of HEK293T scEVs 10\hat{ }5 EVs (n=16) in solution.
(K) Single scEV analysis with Exoview. Schematic of Exoview method that measures +scProtein and tetraspanin content, as well as particle size on single scEVs.
(L) +scProtein fluorescence colocalizes with EV markers in scEVs. HEK293T scEVs were captured by CD81 antibodies and detected with CD81 and CD63 antibodies. +scProtein detection occurred in the blue channel. Fluorescent intensities were used to quantify CD63, CD81 and +scProtein levels while interferometric measurements informed about the size of the scEV. Shown are representative fluorescent images of αCD81 capture spots whereby we identified +scProteins, CD63, CD81 in loaded (scEVs) and unloaded (control EVs) from HEK293T EV samples. Overlap of all markers is visualized in white, overlap of αCD63 and αCD81 is in light grey.
(M) Exogenous loaded +scProtein content in scEVs is EV size dependent. In scEVs, we compared particle size measured with interferometry with +scProtein content through fluorescent intensity. The correlation between scEV size and +scProtein fluorescence was R2=0.81.
(N) Endogenous loaded tetraspanin content is not EV size dependent in scEVs. For tetraspanin content in scEVs, we compared tetraspanin fluorescence with particle size. The correlation of particle size and detection antibodies CD63 or CD81 was 0.32 and 0.34, respectively. +scProtein, CD63 and CD81 were detected in scEVs between 50 nm and 200 nm, with 50 nm being the detection limit of the Exoview method. The linear model is indicated.

Data are presented with mean and SEM (error bars) and analyzed with unpaired t-test. **** and * represent p-values of <0.0001 and <0.5, respectively. R2 represents the statistical measure of how close the data are to the fitted regression line.

FIGS. 2A-L. Membrane glycosylation is crucial for +scProteins to associate with extracellular vesicles.
(A) Biobeads SM-2 (biorad) were not able to pull-down +scProtein. Illustration that recombinant proteins, such as +scProteins, remain in the supernatant when incubated with Biobeads, which are known to interact with hydrophobic lipids.
(B) Effect of Biobeads SM-2 incubation on recombinant +scProtein. Bar graphs represent the percentage of +scProtein fluorescence in Biobeads supernatant (n=6) or Biobeads pellet (n=6) after incubation with a +scProtein suspension without EVs.
(C) Pull-down of scEVs with Bio-Beads SM-2. Illustration that scEV post-Ni-NTA incubation are pelleted by Biobeads due to their membrane content.
(D) Effect of Biobeads SM-2 incubation on scEVs. Bar graphs represent the percentage of +scProtein fluorescence in Biobeads supernatant (n=6) or pellet (n=6) after incubation with scEVs. scEVs were generated with Ni-NTA cleanup and are derived from HEK293T cells.
(E) Illustration of use of Ni-NTA resin to detect +scProtein and EV or liposome association.
(F) +scProteins interact with EVs but not with liposomes. Bar graph represent the +scProtein fluorescence in the Ni-NTA supernantant in following conditions: +scProtein without EVs (no vesicle control, n=4), +scProtein with negatively charged DPPC-PEG(2000)-DSPE-cholesterol liposomes (synthetic liposomes, n=5), and +scProtein with HEK293T EVs (EVs, n=6).
(G) Graphical representation of the negatively charged glycosylated moieties on the surface of EVs as a potential +scProtein loading platform. Sulfated domains of heparan sulfate or oligosaccharides of glycoproteins can be removed by Heparinase [H] and PGNase [P], respectively.
(H) EV loading with +scProtein is inhibited by enzymatic deglycosylation. HEK293T EVs (n per group=6) were treated with PGNase F, Heparinase I, Heparinase II, or Heparinase III and exposed to Ni-NTA affinity resin. Bar graphs represent the +scProtein fluorescence in supernatant post-Ni-NTA pull-down.
(I) scEV loading with +scProteins is EV glycosylation dependent. Bar graphs represent the +scProtein fluorescence in Ni-NTA pellet post-Ni-NTA pull-down shown in H.
(J) Track position of +scProtein in scEVs with deglycosylation assay. Possibility 1 illustrates scEVs as a platform for +scProteins whereby the latter stick to the outer surface. Deglycosylation of scEVs in this possibility 1 results in +scProtein capture by Ni-NTA resin.
(K) Track position of +scProtein in scEVs with deglycosylation assay. Possibility 2 illustrates scEVs as a stable scProtein-EV assembly whereby deglycosylation does not result in Ni-NTA resin capture of +scProtein fluorescence.
(L) Deglycosylation of scEVs does not disrupt +scProtein-EV assembly. Bar graph represents the percentage of +scProteins fluorescent signal in Ni-NTA supernatant vs Ni-NTA pellet in following conditions: only +scProtein (n=4), untreated HEK293T scEVs (n=8), and deglycosylated HEK293T scEVs (n=4). These results confirm possibility 2. HEP=Heparinase.

Data is presented as mean and SEM (error bars) and analyzed with unpaired t-test or one-way ANOVA. ** , *** and **** represent a p-value of ≤0.01, <0.0001 to 0.001 and <0.0001, respectively.

FIGS. 3A-J. Monitoring uptake of supercharged EVs (scEVs) with HEK293T recipient cells. (A) Bodipy TR-scEVs to study the interaction of scEVs with recipient cells.
(A) Monitoring EV uptake and scProtein kinetics with Bodipy TR-scEVs. Cartoon showing that the membrane of scEVs can be labelled with Bodipy TR Ceramide to study the uptake of scEVs by recipient cells. scProtein fluorescent signal can be monitored to study whether +scProtein is protected from cellular degradation when associated with EVs (exposure of scEVs vs scProtein without EVs). Bodipy TR signal increases with increased cellular take up, while scProtein fluorescence decreases with increased cellular degradation.
(B) scEV uptake by cells. Bodipy TR fluorescent signal was monitored to measure difference in uptake between supercharged and non-supercharged EVs (exposure of scEVs vs EVs). HEK293T Bodipy TR-scEVs (scEVs, n=3) and HEK293T BODIPY TR-EVs (EVs, n=3) were incubated with HEK293T cells and Bodipy TR fluorescence was measured in cells. +scProtein (control, n=3) exposure without EVs was monitored as a noise control for Bodipy TR fluorescence. Fluorescence measurements were performed every 10 min for 200 min. Curves indicate are nonlinear regression models for scEVs (R2=0.85) and EVs (R2=0.92). Each datapoint was normalized to its starting value.
(C) scProtein kinetics post-scEV uptake. scProtein fluorescent signal was monitored to measure the cellular degradation between unassociated (free scProtein) and EV-associated scProtein (scEVs). HEK293T Bodipy TR-scEVs (scEVs, n=3) and +scProtein without EVs (scProtein, n=3) were incubated with HEK293T cells and scProtein fluorescence was measured over time. HEK293T Bodipy TR-EVs without scProtein (control, n=3) was monitored as a noise control for +scProtein fluorescence. Fluorescence measurements were performed every 10 min for 200 min. Curves indicate are nonlinear regression models for scEVs (R2=0.92) and +scProtein (R2=0.72). Each datapoint was normalized to its starting value.
(D) Effect of inhibitors on Bodipy TR-scEVs uptake by cells. HEK293T cells were pretreated with DMSO (sham, n=5), v-ATPase blockers −200 nM Bafilomycin A1 (Baf, n=5) and 200 nM Concanamycin A (ConA, n=5), 10 uM lysosomotropic Chloroquine (CHL, n=5), or the 26S proteasome inhibitor 5 uM Bortezomib (Bort, n=5) 1 h before incubation with HEK293T Bodipy TR-scEVs. After 24 h of scEV exposure, Bodipy TR fluorescence in the recipient cells was acquired with flow cytometry.
(E) Blocking scProtein kinetics with inhibitors. Effect of inhibitors on scProtein degradation by cells post-Bodipy TR-scEVs uptake. Conditions are explained in (D). scProtein fluorescence in the recipient cells was acquired with flow cytometry after 24 h of scEV exposure.
(F) Generating scEVs with a different particle size. Separation of Bodipy TR-scEVs based on particle size by serial filtration with different cut-off filters. Each bar was normalized to 100% and represents the volume in the flow through (white) and the volume remaining in the filter (grey).
(G) Particle size does not influence scEV uptake. HEK293T cells were incubated with different sizes of HEK293T Bodipy TR-scEVs. For each particle size scEV subpopulation (n=3) Bodipy TR fluorescence was imaged every 10 min for 24 h. Each datapoint was normalized to the starting value and the residue volume. Curves indicate nonlinear regression models for 100 nm<Y<220 nm (R2=0.75), 30 nm<Y<100 nm (R2=0.30), and 10 nm<Y<30 nm (R2=0.73).
(H) Particle size of scEVs affect stability of scProtein in the recipient cell. +scProtein fluorescence post-scEV uptake by HEK293T cells was measured in conditions explained in (G). Curves indicate nonlinear regression models for scProtein fluorescence for 100 nm<Y<220 nm (R2=0.24), 30 nm<Y<100 nm (R2=0.12), and 10 nm<Y<30 nm (R2=0.21).
(I) Effect of EV origin on scEV kinetics. Bodipy TR-scEVs and BodipyTR-EVs derived from HEK293T (n=3) [autologous EVs], Hela (n=3) [heterologous EVs], GL261(n=3) [heterologous EVs] cells were incubated with HEK293T cells for 200 min (see also FIG. 9). Bars represent Bodipy TR and scProtein fluorescence after 200 min to model scEV uptake and scProtein kinetics, respectively.
(J) Kinetics of supercharged Virus-like particles (scVLPs). VLPs were generated by expressing VSV G (n=3), GAG (n=3), or VSV G/GAG (n=3) in HEK293T cells and labelled with Bodipy TR. scVLP uptake and scProtein kinetics was measured as explained in (I).

Fluorescence in time measurements was normalized to t=0. Data is presented as mean and SEM (error bars) and analyzed with unpaired t-test, Kruskal-Wallis test or one-way anova test. **** , *** , ** and * represent a p-value of <0.0001, <0.001, <0.01, and <0.05, respectively. R2 represents the statistical measure of how close the data are to the fitted regression line.

FIGS. 4A-K. Downstream fate of +scProtein post-scEVs uptake.
(A) scProtein delivered with scEVs leak from the endolysosomal compartments post-scEVs uptake. HEK293T scEVs were exposed for 3 days to HEK293T cells prior to endosomal compartment labelling with Lysotracker Red DND 990 (50 nM). White arrows highlight fluorescent +scProtein signal that is not overlapping with Lysotracker Red.
(B) Lysosomal escape of +scProtein post-scEV uptake by cells. A line profile of Lysotracker Red (top) and +scProtein (bottom) fluorescent intensities observed in the grey square in FIG. 4A were analyzed and represented in the graphs on the right. +scProtein fluorescence graph displayed two peaks of fluorescence that overlap with Lysotracker Red graph and a third one (middle peak) that does not overlap with the Lysotracker Red signal. Scalebar is 20 μm.
(C) Nuclear translocation of NLS-scProtein post-scEVs uptake. HEK293T EVs were loaded with a +scProtein coupled to a nuclear localization signal (NLS-scProtein). Fluorescent images of HEK293T cells exposed to NLS-scEVs for 3 days were screened for NLS-scProtein fluorescence (Fitc channel-cyan) in the cell nucleus. Actin filaments in recipient cells were stained with phalloidin (Cy3 channel). White arrows highlight fluorescent NLS-scProtein signal in nucleus (DAPI channel), which does not colocalize with Cy3. Scalebar is 10 μm.
(D) Nuclear cell fraction isolation to quantify nuclear translocation of NLS-scProtein post-scEVs uptake. Cell nuclei were isolated from HEK293T cells which had been exposed for 3 days to different scEVs, including HEK293T scEVs, HeLa scEVs, GL261 scEVs, VSV G-scVLPs, GAG-scVLPs, and VSV G/Gag-scVLPs (n per condition=3). Bars represent fluorescent intensity of NLS-scProtein from isolated cell nuclei of HEK293T cells exposed to either BVs without scProtein (white) or supercharged BVs (grey).
(E) Functional cargo delivery with scEVs. Plasmid DNA (pDNA) mediated delivery of reporter transgene Nanoluciferase (pDNA-Nanoluc) with pDNA-scEVs. Cartoon illustrating pDNA association with purified EVs through +scProtein to generate pDNA-scEVs. When pDNA-scEVs are taken up by recipient cells, successful delivery of pDNA generates a bioluminescent signal through its encoded Nanoluc transgene.
(F) DNA is protected from DNAse when assembled into pDNA-scEVs. 1 μg pDNA was associated with pDNA-scEVs and used to assess pDNA protection against DNAse I degradation (15 min). DNAse I treated vs untreated pDNA-scProtein and pDNA-scEVs samples were loaded on an 0.75% agarose gel and visualized with GelRed Nucleic Acid dye.
(G) pDNA expression in recipient cells mediated by pDNA-scEVs. pDNA-scEVs generated with 1 μg pDNA and HEK293T EVs were incubated with HEK293T cells for 4 days and compared to conditions lacking either one or two components of the pDNA-scEV assembly (i.e., pDNA, +scProtein, or EVs, n=3 each). Bars represent the bioluminescence measured in the cell media of recipient cells resulting from transgene expression.
(H) pDNA-scEVs mediated delivery with EVs derived from different donor cell species. pDNA-scEVs generated with 1 μg pDNA and HEK293T (n=3), HeLa (n=3), or GL261 (n=3) EVs were incubated with HEK293T cells for 4 days. Bars represent the bioluminescence measured in the cell media of recipient cells resulting from pDNA expression among conditions with pDNA and EVs without +scProtein (white) or with +scProtein (grey).
(I) pDNA mediated delivery with pDNA-supercharged virus-like particles (scVLPs). pDNA-scVLPs generated with 1 μg pDNA and VLPs derived from HEK293T cells expressing VSV G (n=3), GAG-VLPs (n=3), or VSV G/GAG-VLPs (n=3) were incubated with HEK293T cells for 4 days. Bars represent the bioluminescence measured in the cell media of recipient cells resulting from pDNA expression between conditions with pDNA and BVs without +scProtein (white) or with +scProtein (grey).
(J) Nanoluc transgene expression post-pDNA delivery mediated by supercharged lentiviral vectors (scLVVs) transporting a mCherry transgene. pDNA-scLVVs generated with 1 μg pDNA (Nanoluc-transgene) and HEK293T LVVs (mCherry-transgene) were incubated with HEK293T cells for 3 days and compared to conditions lacking one component of the pDNA-scLVV assembly (i.e., pDNA, +scProtein, or LVVs). pDNA-Nanoluc expression was highly increased when the 3 components of the pDNA-scLVV assembly were present. Bars represent the bioluminescence measured in the cell media of recipient cells resulting from pDNA expression (N=5). Different shades of grey represent the increase of bioluminescent signal in time.
(K) Fluorescent images of recipient pDNA-scLVV cells. 72 h post-pDNA-scLVVs exposure of HEK293T cells pDNA-Nanoluc transgene expression is shown (anti-FLAG), with LVV transgene (mCherry) and +scProtein (scProtein). Scalebar represents 10 μm.

Data presented with mean and SEM (error bars) and analyzed with unpaired t-test, or one-way anova test. When needed, data was transformed to log10 to qualify for test assumptions. **** , *** , ** and * represent a p-value of <0.0001, <0.001, <0.01, and <0.05, respectively.

FIGS. 5A-J. Two component delivery with supercharged lentiviral vectors (scLVVs) to single cell demonstrated through bioluminescent reporter activity in vitro and in vivo.
(A) FLEx reports on complex payload delivery with pDNA-scLVVs. Illustration describing that a plasmid (pDNA) encoding for CRE is associated with our supercharging procedure to a lentiviral vector (LVV) encoding for a FLEx reporter. The FLEx reporter encodes for an inverted version of Nanoluc DNA between the LoxP sites (FLEx-OFF). A bioluminescent signal in a recipient cell necessitates delivery of both CRE and FLEx transgenes by our pDNA-scLVV modality to a single cell. CRE acts on the FLEX (FLEx-OFF) reporter transgene to flip Nanoluc in frame with the promotor (FLEx-ON).
(B) pDNA-scLVV delivers both Cre and FLEx transgenes to recipient cells. 1 ug of pDNA was loaded into pDNA-scLVVs whereby the CRE was preceded by the mammalian promoter EF1α(mCre, N=4), which is active in mammalian cells or the bacterial active lac promoter (bCre, n=4), which is inactive in mammalian cells. Graph represents bioluminescence readings over time after pDNA-scLVVs exposure to HEK293T cells.
(C) FLEx activity post-pDNA-LVVs uptake by recipient cells confirmed with qPCR. Schematic representation showing FLEx reporter analysis on genomic DNA with primer pairs distinguishing between the floxed and unfloxed state of the FLEx reporter (ON- vs OFF-state). Agarose gel illustrating the 615 bp floxed amplicon and the 534 bp unfloxed amplicon of the FLEx reporter following 7 days of pDNA-scLVVs exposure. Floxed and unfloxed FLEx amplicons were quantitatively compared with qPCR between cells exposed to pDNA(mCre)-scLVV (n=3) or pDNA(bCre)-scLVV (n=3).
(D) Delivery of multi-component payload to the brain with pDNA-scLVVs. Cartoon describing intracranial injection of the pDNA-scLVVs platform containing pDNA-Cre and LLV-FLEx in Ai9 reporter mice. Successful delivery of pDNA-scLVVs encoded transgenes can be verified with FLEx and Ai9 reporter in striatal cells through activation of nanoluciferase and tdTomato, respectively.
(E) Activation of pDNA-scLVV delivered FLEx reporter in the brain. 80 μm sections were taken for analysis from Ai9 mice that received pDNA(mCre)-scLVV ranging from anterior to posterior brain regions were taken (n=5) to measure Nanoluc bioluminescence as an indicator for FLEx activation. The peak of bioluminescence indicates the injection site.
(F) FLEx reporter activity at pDNA-scLVV injection site. Brain sections at injection site were compared based on bioluminescence between mice exposed to pDNA(mCre)-scLVV (test, n=5), pDNA(bCre)-scLVV (control, n=4), or sham (n=4). Significant differences were only observed at the injection site.
(G) FLEx reporter activity outside pDNA-scLVV injection site. Brain sections at other regions outside injection site were compared similar to (F).
(H) Mouse Ai9 reporter to demonstrate local delivery of pDNA-scLVV transgenes in striatum. pDNA-scLVV recipient cells were identified through GFP encoded by the FLEx reporter and verified for Cre activity through tdTomato fluorescence because of Ai9 reporter activation. GFP and tdTomato were detected with αGFP and αRFP, respectively. Scalebar is 50 μm.
(I) tdTomato expression levels as quantitative measure of Ai9 reporter activity with pDNA-scLVVs in the brain. tdTomato mRNA levels were measured in brain sections at injection site from mice injected with either pDNA(mCre)-scLVV (n=5) or pDNA(bCre)-scLVV (n=4). Data was normalized against GAPDH.
(J) Floxing events to detect Ai9 reporter mice post-pDNA-scLVVs mediated delivery of Cre. Primer sets discriminating between the floxed and unfloxed Ai9 reporter mRNA levels were used as indicated in the cartoon. Floxing events of the Ai9 reporter were quantified in brains sections at injection site of mice injected with pDNA(mCre)-scLVV (n=5) or pDNA(bCre)-scLVV (n=4). Bars represent a ratio between floxed vs unfloxed Ct-values.

Data presented with mean and SEM (error bars) and analyzed with unpaired t-test, or one-way anova test. **** , *** , ** and * represent a p-value of <0.0001, <0.001, <0.01, and <0.05, respectively.

FIGS. 6A-B. Mutagenesis of amino acids in functional site of scGFP alters fluorescent detection. (A) Graph showing the correlation between protein concentration and the fluorescence signal when excited at 485 nm. (B) Graph showing the correlation between protein concentration and the fluorescence when excited at 433 nm. The original scGFP protein is shown as open circles/dashed line while the newly generated scmCerulean3 is displayed as closed circles/solid line.

FIGS. 7A-C. Bulk scEV characterization. (A) Purified EVs assemble with +scProtein to generate scEVs. Top, Schematic of how scEVs were isolated with Ni-NTA resin from a mixture of positive supercharged Protein (+scProtein) and purified EVs. Middle, Green fluorescence under ultraviolet light of solutions that were exposed to Ni-NTA resin. The conditions from left to right are PBS (EV diluent), +scProtein and HEK293T EVs, HEK-293T EVs alone, and +scProtein alone. On the bottom of the tube, we observed the pelleted Ni-NTA resin. Bottom, scEVs after Ni-NTA cleanup could be pulled down with αCD63 affinity beads co-immunoprecipitating +scProtein. On the left single fluorescent signals are shown, including αCD63 immunoaffinity beads alone, with EVs and αCD63-APC, and with HEK293T scEVs alone without αCD63-APC. On the right side αCD63 immunoaffinity beads with HEK293T scEVs and αCD63-APC are shown. Dotted line represents +scProtein loaded EVs. Data was acquired with flow cytometry. (B) Effect of Ni-NTA cleanup on scEV integrity demonstrated with size exclusion chromatography (SEC). Top, SEC aids in resolving unloaded +scProtein from scEVs in solutions, as shown in the schematic. Big particles such as HEK293T EVs were detected in the early SEC fractions, while small particles, such as free +scProtein come off in later SEC fractions. Middle, +scProtein could be detected in both early and late SEC fractions in solutions with +scProtein mixed with EVs (n=3). Bottom, Post-Ni-NTA resin cleanup, we detected the +scProtein only in the early SEC fractions (n=3). Graphs represent green fluorescence derived from the recombinant +scProtein. (C) Effect of Ni-NTA cleanup on scEV integrity demonstrated with Nanosight analysis. Top, Nanosight analysis of HEK293T EVs alone (n=5). Middle, HEK293T EVs and +scGFP (n=5). Bottom, HEK293T EVs and +scGFP after Ni-NTA resin cleanup (n=5). Data are presented with mean and SEM (error bars).

FIGS. 8A-D. Single scEV characterization with Exoview. (A) Number of scEVs (after Ni-NTA cleanup) captured on an Exoview chip through αCD63, αCD81 or αCD9 binding spots compared to an MIgG control spot (n=3). (B) Heatmap of scEVs bound to either αCD63, αCD81 and αCD9 binding spots based on fluorescence of +scProtein and αCD63 and αCD81 detection antibodies. The samples tested were HEK293T EVs without (control HEK293T EVs) and with +scProtein association and Ni-NTA cleanup (HEK293T scEVs). In the latter sample we distinguished EVs that were positive for +scProtein (scEVs) and EVs that were negative for +scProtein. Data represents average colocalization percentage. (C) +scProteins were preferentially loaded into larger sized EVs in the CD63+CD81+subpopulation. Left, We selected EVs captured by the αCD81 spot that were positive for both αCD63 and αCD81 and compared these hits among our different conditions (control HEK293T EVs, HEK293T scEVs loaded with scProtein, HEK293T scEVs not loaded scProtein). We compared the number of events with a certain particle size through interferometric measurements. Right, Graphs representing our three conditions (control HEK293T EVs, HEK293T scEVs loaded with scProtein, HEK293T scEVs not loaded with +scProtein) based on the fluorescence of the αCD63 detection antibody in the CD63+CD81+EVs subpopulation. (D) +scProtein loading capacity of EVs. Y-axis represents the number of +scProteins per EV while the x-axis represents the number of EVs that were loaded with +scProtein cargo. Graph represents the +scProtein fluorescence increase per EV hit on the Exoview chip. Data are presented as the mean with SEM (error bars). Data analyzed with one-way ANOVA with Tukey's multiple-comparisons test. **** represents a p-value of <0.0001.

FIGS. 9A-H. Uptake of scBVs by cells in vitro. (A) Simplified schematic illustration of the scEV labeling procedure with Bodipy TR membrane dye. (B) Bodipy TR membrane dye cleanup procedures. Top, Size exclusion chromatography (SEC) eliminates free Bodipy TR dye. Profile of Bodipy TR fluorescence when a labeled EVs solution was applied to a SEC column. BODIPY TR labelled EVs (n=3) were retrieved in early SEC fractions, while in the later fractions free dye was observed. The left band represents EV fractions, while the right hand band represents protein and free dye fractions. Middle, Free scProtein contaminants were removed with a sized cut-off filter. Our scProtein had a size of 31 kDa, hence it was retained in the 3 nm/30 kDa cut-off filter after serial filtration. The volume of liquid retained in the 3 nm/30 kDa filter is shown. Data is represented as percentage of volume, in which volume in both filter and flow through represent 100%. (C) Bodipy TR-labelled scEVs were bound to a −CD63-bead for detection with flow cytometry. Representative analysis of samples containing EVs with Bodipy TR dye without +scProtein, scEVs without Bodipy TR, and no EVs on the left. A representative sample with scEVs and Bodipy TR can be seen on the right. The dotted line delineates the +scProtein loaded EV sample. (D) HEK293T scEV uptake with inhibitors. HEK293T cells were preincubated with v-ATPase blockers −200 nM Bafilomycin A1 (Baf, n=5), 200 nM Concanamycin A (conA, n=5) or 10 uM of the lysosomotropic Chloroquine (CHL, n=5) together with DMSO (white bars) with or without the 26S proteasome inhibitor −5 uM Bortezomib (grey bars) 1 h before the addition of HEK293T scEVs. After 24 h scEV exposure, Left, Bodipy-TR and Right, +scProtein fluorescence was acquired with flow cytometry. (E) Nanosight analysis of Left, EVs derived from HEK293T, Hela, and GL261 cells show a similar profile and size distribution while Right, Virus-like particles (VLPs) derived from HEK293T with VSV G, GAG, and VSV G/GAG had different size distributions. (F) Uptake of scEVs of different origin by HEK293T recipient cells. Top, HeLa or Bottom, GL261 Bodipy TR labelled scEVs (n=3), +scProtein (n=3) and Bodipy-TR labelled EVs (control, n=3) were incubated with HEK293T cells. When scEVs were taken up by HEK293T cells, Bodipy TR signal increased in red fluorescence as a result of more internalization by HEK293T cells, while cyan fluorescence of +scProtein decreased over time due to degradation. Curves indicate nonlinear regression models; for Top-Left: scEVs R2=0.79 and EVs R2=0.87 ; for Top-Right: scEVs R2=0.50 and scProtein R2=0.73; for Bottom-Left: scEVs R2=0.99 and EVs R2=0.84; for Bottom-Right: scEVs R2=0.08 and scProtein R2=0.73. Bodipy TR (left) and scProtein (right) fluorescence was imaged every 10 min for 200 min. Each datapoint was normalized to the starting value. (G) Bodipy TR-labelled VLPs were generated from HEK293T with VSV G (n=3), GAG (n=3), or both VSVG/GAG (n=3) and incubated with HEK293T cells. Fluorescence was acquired as described in (F). Curves indicate nonlinear regression models; for Top-Left: VLPs/scVLPs R2=0.82, Top-Right: scVLPs R2=0.70, Middle-Left: VLPs/scVLPs R2=0.49, Middle-Right: scVLPs R2=0.40, Bottom -Left: VLPs/scVLPs R2=0.66. Bottom-Right: scVLPs R2=0.51. (H) scEVs and EVs were incubated with HEK293T cells for 24 h and fluorescence was acquired with flow cytometry. Top, Cells that were not exposed to EVs (left dot plot) or scEVs (left dot plot), and Bodipy TR labeled EVs (left dot plot) could be discriminated from cells that had taken up Bodipy TR labeled scEVs (right dot plot). Bottom, After 24 h incubation of HEK293T cells with either Bodipy TR labelled HEK293T (n=3), HeLa (n=3) or GL261 (n=3) scEV or EVs, Bodipy TR (top) and scProtein fluorescence was compared with flow cytometry. Following page, Bar graphs show that Bodipy TR fluorescent signal differed between EV donor cell type (left), while the scProtein fluorescent signal did not (right). Data represent the median fluorescent signal of scEVs compared to the unloaded EV control. Data was analyzed with one-way ANOVA test. **** , *** , ** and * represent a p-value of <0.0001, <0.001, <0.01, and <0.05, respectively. R2 represents the statistical measure of how close the data are to the fitted regression line.

FIG. 10. pDNA and DNAse I. DNAse I treatment degrades pDNA when it is not assembled in pDNA-scEVs. 1 μg pDNA suspensions without or with EVs were incubated for 15 min with DNAse I. Samples were loaded on an 0.75% agarose gel and visualized with GelRed Nucleic Acid dye.

FIGS. 11A-B. CD63 construct expressed in cells. (A) Cartoon explaining exemplary NoMi (Nanoluc Outside and mCherry Inside) design to detect EVs secreted into cell media. NoMi used CD63 as a template to connect the exposed surface (Flag tag and nanoluciferase) with the inner surface (mCherry) of the cell or EVs. TM=transmembrane domain. (B) HEK293T cells expressing the NoMi construct with green fluorescence (copGFP) expressed in cytoplasm and red fluorescence (mCherry) expressed in NoMi-EV generating membrane compartments within the cells. Scale bar represents 20 μm.

FIGS. 12A-C. Exemplary methods for cell selection with Flag tagged NoMi-construct. (A) NoMi operation to isolate NoMi-expressing cells. A Flag tag on the outer surface of the cells can be used to select transduced NoMi-cells through antibody based magnetic bead affinity selection. A NoMi containing single cell suspension is incubated with Flag-Tag affinity beads, whereby NoMi-cells are captured from the cell mixture by their Flag-tag on the cell surface. After washing off non-NoMi expressing cells, NoMi cell-bound beads are cultured in a culturing flask. Following three days of culturing, cells are trypsinized and exposed to a magnet to ensure bead removal from the NoMi cells in the next culturing step. (B) Flag-tag affinity magnetic bead size dictates efficiency of capture of HEK293T cells expressing NoMi construct. Representative fluorescent microscopy images of bead-captured NoMi cells at step 5 of the NoMi operation with 4% agarose beads (top) and Pre-coated Dynabeads (bottom). (C) Significantly more cells (copGFP fluorescence) were captured by 4% Agarose beads when compared to pre-coated Dynabeads. Data represents four experiments (N=4) and are presented as mean with SEM (error bars) and *p<0.01 Unpaired t test. Scale bar is 100 μm in low and 25 μm in high magnification.

FIG. 13A-E. NoMi construct enables EV purification from complex biofluids. (A) NoMi operation to isolate NoMi-expressing EVs. NoMi construct expressing HEK293T cells expose a Flag tag to the outer surface, a characteristic adopted by their NoMi-EV progenies. This affinity tag can be used to isolate NoMi-EVs. We collected the conditioned media after 3 days of culturing NoMi-cells, depleted the cells by centrifugation and concentrated the media with a 100 kDa cut-off filter. After incubation of Flag-Tag affinity beads, NoMi-EVs get captured from the concentrated media by their Flag-tag exposure. The bead bounded NoMi-EVs are washed to eliminate non-NoMi-EV constituents when the suspension is under a magnetic field. Elution with a Flag-Tag (DYDKDDDK, SEQ ID NO:8) peptide ensures elution of NoMi-EVs and generates a dense NoMi-EV suspension. The beads are removed from the NoMi-EV suspension by a magnet. (B) Elution of EVs with Flag-Tag peptide enables recovery of 75% of EVs from the magnetic beads determined with bioluminescent nanoluciferase signal in NoMi (n=4). (C) Schematic of how EVs can be characterized based on their particle size with size exclusion chromatography (SEC). Big particles, including EVs, elute first while smaller particles such as proteins elute later. This method can be used to verify the presence of contaminating protein in EV suspensions. (D) EV isolation of non-NoMi EVs. Top, Schematic of how conditioned media of HEK293T cells was concentrated with a 100 kDa cut-off filter and brought on a SEC column before being characterized based on protein content. Bottom, The predominant SEC peak was observed in the free protein fractions, while little signal was detected in the fractions in the particle size range where we expect to retrieve EVs. This measurement was performed with a protein assay and demonstrates the difficulty to resolve EVs from free protein. (n=3) (E) NoMi-EVs isolated with NoMi operation were brought on SEC column. Top, Schematic showing that prior to elution, bead captured NoMi-EV were either detergent (n=2) or sham (n=2) treated prior to washing with PBS and elution with Flag-tag peptide. Bottom, When SEC-resolved, sham treated intact-NoMi-EVs were predominantly retrieved in the early EV particle size range fractions, while the detergent treated lysed-NoMi-EVs were found in the later free protein size range fractions. Detection of our NoMi construct was performed with NoMi-Nanoluc bioluminescence.

FIGS. 14A-B. Detection of rare brain derived NoMi-EVs in the blood compartment of mice. Left, Intracranial injection of lentivirus vector encoding NoMi-construct within the striatal region. Right, A spatial gradient of NoMi-Nanoluc signal was visualized through coronal sections with a peak of luminescence corresponding to the injection site and being lower to both anterior and posterior regions (N=1 control and N=3 NoMi). Left, NoMi-labelling method ensures NoMi-EV isolation from serum upon RNA extraction from NoMi-EVs pulled down with Flag-tag beads and Digital droplet PCR (ddPCR). Right, Confirmation of brain-derived NoMi-EV isolation and detection from serum with NoMi-method based on copGFP mRNA detection by ddPCR (N=3 control and N=6 NoMi mice). Data are presented as the mean with SEM (error bars) *p<0.05. Unpaired t test.

FIGS. 15A-C. Implanted NoMi-hNPCs and hNPCs derived EVs are recovered from mouse brain. Protocol of recovering of 14-day implanted hNPCs from a mouse brain with the NoMi-method. (B) CopGFP amplicon is detected in NoMi-hNPCs recovered 14 days after transplantation, after coronal sectioning and proteolytic digestion followed by incubation with anti-flag beads (N=4). (C) Brain-derived EVs were isolated from serum 7 and 14 days after NoMi-hNPCs implantation using the beads-based method. CopGFP amplicon in NoMi-EVs was detected by ddPCR with increasing amounts from 7 to 14 days (N=4, data normalized against GAPDH). Data are presented as the mean with SEM (error bars).

FIG. 16. A schematic illustration of an exemplary CRISPR-editing reporter construct as described herein. Gene editing events can be detected through EVs by a transgenic construct without the need for lysing the gene edited cell. The NoMi construct can be modified to encode upstream of the tags a genomic DNA target sequence for detection of a desired gene editing event is incorporated. The latter generates a premature stop codon withholding expression of the affinity and luciferase tags. Only when the desired gene editing event occurs, the stop codon is corrected and overcomes truncations by genomic nonsense, missense or frameshift mutations in the reporter. Once the gene edit correct the construct, the abilities described for the NoMi construct are valid. In essence, a gene edit generates a bioluminescent signal, as it then allows the CD63 tethered luciferase reporter to be translated. The CD63 construct is incorporated in EVs secreted by the gene edited cell. Construct-containing EVs can be isolated and purified by the affinity tag exposed on the surface of the EVs and detected by bioluminescence.

FIG. 17. CRISPR reporter activation reports on gene editing efficiency. In (A) a plasmid encoding Cas9, sgRNA targeting the reporter and mCherry are expressed in reporter cells, while in (B) a a homology damage repair DNA molecule next to plasmid encoding Cas9, sgRNA targeting the reporter, and mCherry are expressed in reporter cells. Mcherry reports on the expression of cas9 and sgRNA (bottom left), eGFP expression reports on the expression of the crispr reporter (bottom middle) while bioluminescence expresses the gene editing efficiency (bottom right). Only if both mcherry and gfp are expressed in cells, bioluminescence is generated. With condition (B) we generated higher bioluminescent signal than with (A).

FIGS. 18A-B. Activation of CRISPR reporter with supercharged EVs (A) Supercharged EVs containing Cas9 and sgRNA activate CRISPR-editing reporter. Schematic representation that EVs loaded through supercharging with only synthetic sgRNA do not activate the CRISPR Reporter [TOP]. Schematic representation that EVs loaded with Cas9 recombinant protein and synthetic sgRNA through supercharging activate the CRISPR Reporter [BOTTOM]. (B) Exposing CRISPR reporter cells to Cas9/sgRNA-scEVs, generated bioluminescent signals indicating that the CRISPR reporter was activated by the gene editing events of scEVs induced in recipient cells. Conditions tested are explained in (A).

### DETAILED DESCRIPTION

Cell-derived biovesicles (BVs) comprise a large group of bioentities present in the extracellular space ranging from extracellular vesicles (EVs), including exosomes and microvesicles¹, viral-like particles to enveloped viruses², including lentiviral vectors (LVVs). The common elements among all these BV types are their natural capacity to incorporate biomolecules (endogenous cargo or endocargo) from a parent cell, their subcellular scale (nano to micro), their release into the extracellular space, and protection of their luminal contents by a lipid membrane^{3.4}.

Non-infectious EVs have gained more and more attention as a potential therapeutic-delivery vehicle option⁵⁻⁷. EVs have the potential to counter major drawbacks of virus-based vectors, such as immunogenicity, potentially allele-disruptive genome integration, small capacity for additional non-viral encoded biocargo, and potential cytotoxicity. Biomarker studies have demonstrated that the host tolerates high numbers of EVs produced by virtually every cell in the body in biofluids and extracellular spaces, including both from diseased and normal tissues⁸. Limiting factors in EV therapeutic development are our understanding of defined mechanisms of biomolecule sorting from the donor cells into EVs and their fate in recipient cells^{3,9}. The bioactivity of EV cargo acting upon recipient cells has been the subject of debate in recent years, due in part to experimental limitations at the single EV level¹⁰ and quantitation of functional cargo¹¹. The overall observed effect of the EV endocargo in recipient cells is seemingly low and might need additional boost signals to improve therapeutic relevance or multiple EV exposures.

Here, we explore a strategy that is not limited by the donor cell's ability to package a desired payload into EVs. This avenue is called exogenous EV loading, which others have pursued for loading synthetic RNAs and proteins with lipofection agents or electroporation^{12,13}. Our strategy exploits synthetically reprogrammed proteins with positively charged amino acids, such as arginine and lysine, exposed on the outside of the protein structure, while their functional amino acids remain unchanged from their parental structure¹⁴. The biophysical properties of these positively supercharged proteins (+scProteins) enable association with and migration through the cell membranes of living cells¹⁵. Here we demonstrate that +scProteins utilize EV properties to load negatively charged cargo, including DNA and RNA species, as well as fused proteins and aid in the functional delivery of the these latter.

In some of the present methods and composition, +scProteins and EV, VLP, and/or LVV membranes are used to achieve combinatorial functional delivery of cargo to recipient cells. Larger EVs were more prone to +scProtein loading compared to smaller ones, indicating that more luminal volume and interaction surface promote incorporation of +scProteins. The surface of EVs consists of negatively charged lipids, such as ceramides²⁴ and phosphatidylserine³⁴ restricting association with negative supercharged scProteins (−scProteins) and non-supercharged proteins (scaffold). This ensures that larger membrane-encompassed vesicles exert a higher negative charge (from −12.3.0 mV to −16.0 mV), compared to smaller EVs (−9.0 m V to −12.3 mV) and non-membranous exomeres (−2.7 mV to −9.7 mV)³⁵. As an important factor in cargo uptake by EVs, we noticed that our +scProtein did not associate with −11 mV synthetic liposomes in contrast to SEC-purified EVs. This observation is in line with earlier reports that the charge of lipids is less important for docking of Arg-rich peptides in contrast with sugar moieties on biomembranes³⁶. It is known that the EV surface contains heavily glycosylated proteins that influences their uptake by cells³⁷. EV adopt sugars from their originating both plasma membrane and endocytic cell membrane compartments³⁸. In this regard, the extracellular domain of tetraspanins are equipped with N-linked glycosylation sites³⁹ important for endocytic membrane trafficking^{40,41}. We've demonstrated that deglycosylated EVs were not able to integrate +scProteins and a high number of tetraspanins on the EV surface is propitious for +scProtein association. In terms of the % EVs loaded in a SEC purified EV sample, we expect that our supercharging method could be improved by +scProtein loading of EV subpopulations rich in tetraspanins such as CD63⁺CD81⁺EVs, with large particle size, with a high glycosylation status, and with a negative surface charge.

Supercharging of HEK293T EVs did not influence uptake but did increase +scProtein half-life following uptake by HEK293T cells. Improving scEV uptake through using scEVs from different donor cell sources did not increase +scProtein levels when exposed to the same cell type, indicating the scProtein half-life is dependent on processes dictated by the recipient cell. Through lysotracker red experiments scEVs were found to be taken up into low pH cell compartments, indicating that scEVs are exposed to endolysosomal conditions. Without wishing to be bound by theory, it was hypothesized that the stability of scEV assembly inside cells aids in the scEV-mediated delivery of pDNA and other cargo to a recipient cell. Supercharging of proteins protects them against proteolysis and other physical stresses, such as denaturation by temperature or denaturation and aggregation by chemicals like 2,2,2-trifluoroethanol^{15,30}. The resilience to many hazardous factors and tolerance of scProteins⁴² compared to amphipathic cell penetrating peptides⁴³ makes them ideal in delivery of associated cargo into a living cell or organism. As demonstrated herein, assembly formation through supercharging of EVs protects the EVs against deglycosylation and protects their cargo against DNAse activity and degradation in recipient cells. Delivery of scEV assemblies containing a higher level of +scProteins, such as large scEVs (220 nm to 100 nm) compared to smaller scEVs (<100 nm) was accompanied with an increased half-life of cargo. Longer stability implies longer interaction with the endosomal compartments, therefore boosting the +scProteins ability to escape from endosomal compartments^{44,45}. We confirmed nuclear translocation of the +scProtein, as well as delayed cytosolic +scProtein degradation, using the 26S proteasome inhibitor Bort in combination with Chloroquine (CHL), Bafilomycin A1 (Baf), and concanamycin A (conA). More importantly, pDNA delivery and expression after cell uptake was increased with pDNA-scEVs compared to +scProtein and pDNA alone.

Formation of pDNA-scEVs is built upon the potential of +scProteins to adhere to/form complexes with genetic material mainly through their lysine residues⁴⁶, while at the same time, protecting the associated DNA from degradation¹⁵. We utilized this +scProtein property to piggyback DNA-scProtein complexes for entrance into EVs. Nanoluc expression in HEK293T cells confirmed transgene delivery by pDNA-scEVs through the detection of bioluminescence. We adapted this readout to corroborate whether supercharging of BVs might be applicable for delivery of multiple types of biomolecules through scLVVs. pDNA-scLVVs generated a nanoluc signal whereby two components CRE and a FLEx-OFF reporter were delivered to the same cell generating bioluminescent readout. The LVV component provided viral RNA encoding a non-active floxed reporter, while the pDNA component encoded a CRE enzyme able to activate the floxed reporter. Apart from nanoluc activity, secondary downstream analysis with qPCR confirmed FLEx reporter editing by Cre. This functional delivery of multiple biomolecule types by means of a single carrier was not only shown in cell culture but substantiated by activation of the Ai9 reporter in mouse brain cells.

Nanoscopic vesicles derived from cells in culture provide a valuable route for supercharging to enhance cargo loading, cellular uptake and functional delivery of cargo. Delivery of multiple types of biomolecules⁴⁷ is a highly valuable tool for next-generation research and modern medicine⁹. The supercharging methods described herein can overcome some of the hurdles seen with packaging EVs through natural and transgenic routes with donor cells, including payload inconsistency, reduced options for multicargo transport, limited control over the loading process, and the need for oversaturating recipient cells with loaded EVs to achieve functional responses⁴⁸.

Described herein are customizable BV, and BV-loading technique that can be:
a two-step procedure (approx. 1 hour),
easily scalable (for in vitro and in vivo purposes),
occurs under physiological conditions (no need for compromising the vesicle integrity or introducing non-physiologic pH, osmotic changes or membrane disruption by detergents),
very efficient (focuses on intact membrane vesicles and not on free protein contaminants),
applicable to all natural glycosylated enclosed biovesicles (e.g., EVs, LVVs or VLPs),
applicable to every BV source (no need for transgenic EV-donor cells),
delivery efficacy of pDNA is tunable by varying vesicle properties, and/or
serves as combinatorial carrier of different types of biomolecules including nucleic acids and proteins, e.g., LVV transgene or pDNA, RNA species and proteins, that are functional in the recipient cell.

Thus, provided herein are compositions comprising positively supercharged proteins (+scProteins), membrane-bound vesicles (EV, VLP, and/or LVV membranes), and cargo, and methods of use thereof to deliver the cargo to the intracellular space of target cells.

### Biovesicles

The present methods and compositions can include the use of any glycosylated enclosed biovesicles (i.e., naturally-derived biovesicles, such as extracellular vesicles (EVs), lentiviral vectors (LVVs), or virus-like particles (VLPs), e.g., GAG-VLPs, VSVG-VLPs GAG/VSV G-VLPs). VSVG VLPs are described, e.g., in Campbell et al., Mol Ther. 2019 Jan 2;27(1): 151-163. GAG GLPs are described, e.g., in Ashley et al., Cell. 2018 Jan 11;172(1-2):262-274.e11. In general, the exosomes are about 30 nm-150 nm; microvesicles are about 100nm-1 um; and lentiviral vectors are about 80-120 nm in diameter. The biovesicles include a bilayer, e.g., similar to a cell membrane, that surrounds an inner aqueous lumen that can contain soluble components. The bilayer is surrounded by a glycocalyx comprising membrane-anchored negatively charged glycoproteins. For example, extracellular vesicles can be comprised of a phospholipid bilayer (and associated glycocalyx) obtained from a donor cell, e.g., an animal cell, e.g., a mammalian cell, or a bacterial cell, comprising various negatively charged glycoproteins. See, e.g., Thery et al., Nat Rev Immunol. 2009 Aug;9(8):581-93; Wang et al. Nature Communications 9, 1-7 (2018); Lainscek et al. ACS Synthetic Biology 7, 2715-2725 (2018)). Methods known in the art for obtaining biovesicles from donor cells can be used. For example, the biovesicles can be obtained from culture media in which donor cells, e.g., primary or immortalized cultured cells, are maintained in vitro. Wang et al., STAR Protocols, Volume 2, Issue 1, 19 March 2021, 100295. Biovesicles can also be obtained from biofluids, e.g., blood, urine, cerebrospinal fluid (CSF), tears, saliva, breast milk, ascites, etc. or from tissues (e.g., from brain, lung, tumor, or adipose tissue-derived mesenchymal stem/stromal cells; see, e.g., Hurwitz et al., J Vis Exp. 2019 Feb 7; (144): 10.3791/59143; Kaur et al., Int. J. Mol. Sci. 2021, 22, 11830; Lee et al., Int J Mol Sci. 2020 Jul; 21(13): 4774). Biovesicles can be concentrated or isolated from a biological sample using size exclusion chromatography, density gradient centrifugation, differential centrifugation, nanomembrane ultrafiltration, immunoabsorbent capture, affinity purification, microfluidic separation, anion exchange exodisk, asymmetrical flow field-flow fractionation, tangential flow and/or gel permeation chromatography (for example, as described in U.S. Pat. Nos. 6,899,863 and 6,812,023), iodixanol and sucrose density gradients, organelle electrophoresis (for example, as described in U.S. Pat. No. 7,198,923), magnetic activated cell sorting (MACS), with a nanomembrane ultrafiltration concentrator, polymer-based precipitation, or immunological separation; see, e.g., Yakimchik, Exosomes: isolation and characterization methods and specific markers, 2016-11-30, dx.doi.org/10.13070/mm.en.5.1450, and references cited therein. An exemplary polymer based exosome precipitation system is the ExoQuick from System Biosciences. Various combinations of isolation or concentration methods can be used. See, e.g., Witwer et al., J Extracell Vesicles. 2021 Dec; 10(14):e12182 for EV nomenclature, sample collection and pre-processing, EV separation and concentration, characterization, functional studies, and reporting requirements/exceptions.

+scProteins

The present methods and compositions include positively supercharged proteins, which have, or have been engineered to have, a surface charge of at least +11; in some embodiments, the protein is at least +15, +20, +25, +27, +30, +32, +35, +36, or +38. Surface charge can be predicted using bioinformatics, including RaptorX (Xu et al., Nature Machine Intelligence 3:601-609 (2021), available at raptorx.uchicago.edu) and I-TASSER(Yang et al., Nature Methods, 12: 7-8 (2015), and Yang et al., Nucleic Acids Research, 43: W174-W181 (2015), available at zhanggroup.org//I-TASSER/) that can be used to generate a Predicted Solvent Accessible (PSA) Amino Acids based on a or PSA score. Exemplary positively supercharged proteins can include +scmCerulean3 and +scGFP (sequences provided below). The surface charge of a protein can be increased by the addition of amino acids with a positive charge, e.g., amino acids that are presented on the surface of the protein. For example, the solvent exposed surface of a beta-barrel protein, such as mCerulean3, can be engineered to contain increased levels of positively charged amino acids—Lysine, Arginine or Histidine—on the outside of the barrel. Proteins with beta-barrel structures include human retinol-binding protein, porins, lipocalins, and translocases. Other enzymes or proteins with a functional site can also be supercharged if the positively charged amino acid residues are incorporated at distal sites to the catalytic cleft on the solvent exposed protein surface. Example of such functional proteins include Cas9, Cre, Talen and zinc finger nucleases.

In some embodiments, the +scProteins comprise +scmCerulean3 (sequence provided below). Also provided herein are the +scProteins themselves, e.g., isolated recombinant +scmCerulean3, as well as nucleic acids encoding the +scmCerulean3, vectors comprising the nucleic acids, and host cells comprising and optionally expressing the +scmCerulean3. A vector is a nucleic acid molecule capable of transporting another nucleic acid to which it has been linked and can include a plasmid, cosmid or viral vector. The vector can be capable of translation (RNA), autonomous replication (RNA or DNA) or it can integrate into a host DNA. Viral vectors include, e.g., replication defective retroviruses, adenoviruses and adeno-associated viruses.

A vector can include a +scmCerulean3 nucleic acid in a form suitable for expression of the nucleic acid in a host cell. Preferably, the recombinant expression vector includes one or more regulatory sequences operatively linked to the nucleic acid sequence to be expressed. The regulatory sequences can include promoters, enhancers and other expression control elements (e.g., polyadenylation signals). Regulatory sequences can include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by +scmCerulean3-encoding nucleic acids as described herein (e.g., +scmCerulean3 proteins, fusion proteins, and the like). The expression vectors described here for +scmCerulean3 can also be used for other purposes herein.

### Exemplary Protein Sequences:

AA sequence eGFP=GFP

(SED ID NO:1)
MVSKGEELFTGVVPILVELDGDVNGHKFSVRGEGEGDATNGKLTLKFI
CTTGKLPVPWPTLVTTLTYGVQCFSRYPDHMKRHDFFKSAMPEGYVQER
TISFKDDGTYKTRAEVKFEGDTLVNRIELKGIDFKEDGNILGHKLEYNF
NSHNVYITADKQKNGIKANFKIRHNVEDGSVQLADHYQQNTPIGDGPVL
LPDNHYLSTQSVLSKDPNEKRDHMVLLEFVTAAGITHGMDELYK

AA sequence positive supercharged eGFP=+scGFP

(SED ID NO:2)
MASKGERLFRGKVPILVELKGDVNGHKFSVRGKGKGDATRGKLTLKF
ICTTGKLPVPWPTLVTTLTYGVQCFSRYPKHMKRHDFFKSAMPKGYVQE
RTISFKKDGKYKTRAEVKFEGRTLVNRIKLKGRDFKEKGNILGHKLRYN
FNSHKVYITADKRKNGIKAKFKIRHNVKDGSVQLADHYQQNTPIGRGPV
LLPRNHYLSTRSKLSKDPKEKRDHMVLLEFVTAAGIKHGRDERYK

AA sequence negative supercharged eGFP=−scGFP

(SED ID NO:3)
MSKGEELFDGVVPILVELDGDVNGHEFSVRGEGEGDATEGELTLKFIC
TTGELPVPWPTLVTTLTYGVQCFSDYPDHMDQHDFFKSAMPEGYVQERT
ISFKDDGTYKTRAEVKFEGDTLVNRIELKGIDFKEDGNILGHKLEYNFN
SHDVYITADKQENGIKAEFEIRHNVEDGSVQLADHYQQNTPIGDGPVLL
PDDHYLSTESALSKDPNEDRDHMVLLEFVTAAGIDHGMDELYK

AA sequence of supercharged mCerulean3=+scmCerulean3

(SED ID NO: 4)
MVSKGERLFRGKVPILVELKGDVNGHKFSVRGKGKGDATRGKLTLKF
ICTTGKLPVPWPTLVTTLSWGVQCFARYPKHMKRHDFFKSAMPKGYVQE
RTISFKKDGKYKTRAEVKFEGRTLVNRIKLKGRDFKEKGNILGHKLRYN
AIHGKVYITADKRKNGIKAKFGLNCNVKDGSVQLADHYQQNTPIGRGPV
LLPRNHYLSTRSKLSKDPKEKRDHMVLLEFVTAAGIKLGRDERYK

AA sequence of wild type mCerulean3

(SEQ ID NO:5)
MVSKGEELFTGVVPILVELDGDVNGHKFSVSGEGEGDATYGKLTLKFI
CTTGKLPVPWPTLVTTLSWGVQCFARYPDHMKQHDFFKSAMPEGYVQER
TIFFKDDGNYKTRAEVKFEGDTLVNRIELKGIDFKEDGNILGHKLEYNA
IHGNVYITADKQKNGIKANFGLNCNIEDGSVQLADHYQQNTPIGDGPVL
LPDNHYLSTQSKLSKDPNEKRDHMVLLEFVTAAGITLGMDELYK

In some embodiments, the +ScProtein is at least 80%, e.g., at least 85%, 90%, 95%, 97%, or 98% identical, or is 100% identical, to a reference sequence provided herein (e.g., SEQ ID NO: 2 or 4). To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

In some embodiments, additional positive charge can be added by the addition of one or more positively charged peptides such as a nuclear localization signal (NLS). Exemplary NLS include SV40 NLS (PKKKRKV; SEQ ID NO:6) or nucleoplasmin NLS (KRPAATKKAGQAKKKK; SEQ ID NO:7). Other NLSs are known in the art; see, e.g., Cokol et al., EMBO Rep. 2000 Nov 15; 1(5): 411-415; Freitas and Cunha, Curr Genomics. 2009 Dec; 10(8): 550-557. The +scProteins can be produced recombinantly using methods known in the art. For example, +scProteins can be expressed in E. coli, insect cells (e.g., using baculovirus expression vectors), yeast cells or mammalian cells. Suitable host cells, methods of expression, and methods of purification are known in the art, see, e.g., Goeddel, (1990) Gene Expression Technology: Methods in Enzymology 185, Academic Press, San Diego, CA; Wingfield, Curr Protoc Protein Sci. 2015; 80: 6.1.1-6.1.35; Young et al., Biotechnol J. 2012 May;7(5):620-34; Rosane and Ceccarelli, Front. Microbiol., 17 April 2014, doi.org/10.3389/fmicb.2014.00172. In some embodiments, the +scPRotein includes an affinity tag attached at the N- or C-terminus, and the tag is used in purification; see, e.g., Mishra, Curr Protein Pept Sci. 2020;21(8):821-830. Affinity tags can include FLAG, hemagglutinin, myc, streptavidin, polyhistidine (e.g., hexahistidine), GST, and so on. Alternatively, the recombinant expression vector can be transcribed and translated in vitro, e.g., using T7 promoter regulatory sequences and T7 polymerase.

### Cargo

“Cargo” can include biomolecules, e.g., nucleic acids (e.g., DNA, RNA), a combination of DNA and RNA, Ribonucleoproteins (RNPs), a combination of DNA and proteins, proteins, or natural or synthetic small or large molecules. The cargo can be used or intended for use, e.g., as a therapeutic or diagnostic; in some embodiments, the cargo is used for the applications of genome editing, epigenome modulation, and/or transcriptome modulation. In some embodiments, plasmids of up to 11, 12, 13, 14, or 15 kB are delivered with this method. Several different kinds of cargo can be included, e.g., RNA or DNA, proteins, and small or large molecules.

One of skill in the art will appreciate that these are examples and are non-limiting. In some embodiments, the cargo is for genome editing, epigenome modulation, and/or transcriptome or translational modulation, and includes delivering proteins, or nucleic acids encoding proteins, that can effect genome editing, epigenome modulation, and/or transcriptome or translational modulation, e.g., Clustered Regularly Interspaced Palindromic Repeat (CRISPR) based nucleases or nickases, base editors, and other proteins that comprise CRISPR based nucleases to direct an effector protein to target DNA, e.g., that comprise a CRISPR based nuclease (as used herein, “CRISPR based nucleases” includes proteins that have no nuclease activity, or have only nickase activity). Exemplary CRISPR based nucleases include Cas9 (e.g., SpCas9 or SaCas9), xCas9, Cas12a (Cpf1), Cas13, and others. RNA in this context can be, e.g., a single guide RNA (sgRNA), CRISPR RNA (crRNA) and tracrRNA (e.g., for use with CRISPR based nucleases, and/or mRNA coding for cargo. Cargo developed for applications of genome editing also includes, e.g., nucleases and base editors. CRISPR based nucleases are described, for example, in United States Patent Publications U.S. Pat. No. 8,697,359B1; US20180208976A1; International Publications WO2014093661A2; WO2017184786A8; and Anzalone et al., Nature Biotechnology 38:824-844 (2020). Other nucleases include, e.g., FokI and Acul zinc finger nucleases (ZFNs) and Transcription activator-like effector nucleases (TALENs). ZFNs are described, for example, in United States Patent Publications 20030232410; 20050208489; 20050026157; 20050064474; 20060188987; 20060063231; and International Publication WO 07/014275. TALENs are described, for example, in United States Patent Publication U.S. Pat. No. 9,393,257B2; and International Publication WO2014134412A1. Base editors or prime editors can also be delivered, include any CRISPR based nuclease orthologs (wild type, nickase, or catalytically inactive (CI)), e.g., fused at the N-terminus to a deaminase or a functional derivative thereof with or without a fusion at the C-terminus to one or multiple uracil glycosylase inhibitors (UGIs) using polypeptide linkers of variable length. Base editors are described, for example, in United States Patent Publications US20150166982; US20180312825; U.S. Pat. No. 10,113,163; and International Publications WO2015089406; WO2018218188; WO2017070632; WO2018027078; and WO2018165629. Prime editors are described, e.g., in WO2020191248, Anzalone et al., Nat. Biotechnol., 38: 824-844 (2020); Anzalone et al., Nature, 576: 149-157 (2019); Song et al., Nature Communications 12:5617 (2021), and Chen et al., Cell 184(22): 5635-5652.e29, 28 October 2021. sgRNAs can optionally be complexed with genome editing reagents during production within producer cells. Cargo could refer to AAV (e.g., AAV protein capsid and ITR-flanked DNA cargo). Cargo designed for the purposes of epigenome modulation can include CRISPR based nucleases, zinc fingers (ZFs) and TALEs fused to an epigenome modulator or combination of epigenome modulators or a functional derivative thereof connected together by one or more variable length polypeptide linkers. Cargo designed for the purposes of transcriptome editing can include CRISPR based nucleases or any functional derivatives thereof or CRISPR based nucleases or any functional derivatives thereof fused to deaminases by one or more variable length polypeptide linkers.

The cargo can also include any therapeutically or diagnostically useful nucleic acid, DNA, RNA, protein, RNP, or combination of DNA, protein and/or RNP. See, e.g., WO2014005219; U.S. Pat. No. 10,137,206; US20180339166; U.S. Pat. No. 5,892,020A; EP2134841B1; WO2007020965A1. Exemplary RNAs can include miRNAs, mRNAs, small guide RNA (sgRNA), long non-coding RNAs (IncRNAs), and In some embodiments the cargo is an inhibitory nucleic acid, e.g., antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

Preferably, the cargo is complexed with the +scProtein, e.g., by a charge interaction (e.g., the cargo is negatively charged), or by being conjugated to the +scProtein, e.g., by a chemical bond or other affinity interaction, or (in the case of protein cargo) by being expressed as a fusion protein with the +scProtein. In some embodiments, the cargo comprises a protein for genome editing or epigenome modulation, and the +scProtein can be fused to the N or C terminus of the cargo, optionally with a polypeptide linker therebetween. The cargo complex then flips/is loaded across the EV membrane.

Methods of Loading the Cargo into Biovesicles

Further provided herein are methods for loading cargo into nanosized cell membrane derived biovesicles as described herein. The methods including contacting a sample comprising biovesicles with +scProtein/cargo complexes, under conditions that allow the +scProtein/cargo complexes to be taken across the lipid bilayer into the biovesicles. The methods can include mixing the +scProteins and the cargo to form +scProtein/cargo complexes, e.g., in cases where the cargo and +scProteins are not expressed as a fusion protein. As one example, a fusion protein comprising a CRISPR based nuclease (or a protein comprising a CRISPR based nuclease such as a base editor) linked to a +scProtein as described herein is expressed and purified, e.g., in a bacterial or mammalian expression system, and then mixed with a desired guide RNA, and formation of ribonucleoprotein (RNP) complexes comprising the guide RNA and CRISPR based nuclease is allowed to occur. The RNP complexes are then combined with biovesicles as described herein, and complexes are translocated into the biovesicles into the biovesicle lumen. The methods can include contacting the biovesicles with +scProtein/cargo complexes for at least 15 minutes, e.g., at least 1 hour, 2 hours, 4 hours, 6 hours, 12 hours, 18 hours, or 24 hours, e.g., up to 24, 36, or 48 hours or more. In some embodiments, where the methods include contacting the biovesicles with +scProtein/cargo complexes for 15 minutes to 2 hours, the contacting is performed at 25-27° C.; where the contacting step is performed for longer, e.g., for 18-24 hours, the contacting is performed at about 4° C.

The methods can further include a step of purification to remove any complexes that are not taken into the biovesicles. For example, where the biovesicles containing the +scProteins and/or cargo include an affinity tag exposed on the membrane surface, immunoaffinity purification using an antibody directed to the tag, e.g., affixed to beads or a solid surface, can be used.

Generally speaking, the loading methods can be performed under physiological or near-physiological conditions, e.g., at a pH of 6.5-8, or 6.5-7.5; in the absence of detergents (e.g., Tween, triton ×100, digitonin, and/or saponin); in isotonic solutions (e.g., 280-320 mOsm/L, preferably 280-315, 280-312, 300-312, or 300-310 mOsm/L, for biovesicles derived from mammals), or in solutions that are not hypotonic (e.g., at least 280, 285, 290, 295, or 300 mOsm/L). In some embodiments, the methods do not include sonication or other mechanical disruption of the biovesicles.

### Methods of Use

In some embodiments, the biovesicles are obtained from a subject who is in need of a treatment that would beneficially be delivered using a method described herein, and the methods can include obtaining biovesicles from the subject, loading the biovesicles with +scProtein/cargo complexes, and then re-administering the loaded biovesicles back to the subject, wherein the cargo is a therapeutic agent that is useful in treating the subject. Thus provided are methods of delivering a cargo, e.g., as described herein, e.g., a cargo comprising a therapeutic or diagnostic agent, to a subject in need thereof.

### Reporter System for Gene-Editing Events in Cells

Gene editing is a very powerful technique, but it is somewhat hampered by biological and technical requirements. For example, all components (gene editing enzyme, sgRNA, genomic target sequence) need to be spatially and temporally present/expressed in one cell. Therefore, gene editing is often an inherently low occurrence event. Rare cells that underwent the desired mutation are hard to select out of a pool of wild type cells. Gene editing is often impaired by off-target effects, and genomic repair mechanisms post-cutting by Cas9 and Cas9-like gene editing enzymes can induce undesired frame-shifts or deletions.

To counter these restrictions, single cell sorting is commonly used to select out the desired genetically engineered clones. In this technique, each sorted single cell has to be grown to a high number of cells so as to lyse and subsequently isolate gDNA for sequencing. The latter is necessary to retrieve cells with only the desired genomic correction and low-to-no off-target effects in other regions of the genome. This has proven to be a lengthy method that is concomitant with the loss of many appropriately gene-edited cells.

To facilitate this process, we designed a construct that doesn't require flow cytometry sorting, reports if gene editing events have occurred in a pool of unsorted cells, and preselects for cells that have a significantly higher chance to contain the desired correction in the genome. To accomplish these premises, we designed a transmembrane construct, e.g., comprising a transmembrane domain such as CD63 tetraspanin, that contains a fluorescent tag, bioluminescent tag, and an affinity tag. Expression of these tags is interrupted by a nucleotide sequence complementary to the target gRNA sequence. Our data confirms that when the novel CD63 construct is expressed, the transgenic protein is present in the cell membrane and in extracellular vesicles. On the extracellular side of the membrane the bioluminescent tag and the affinity tag are present while on the intracellular side a fluorescent and/or bioluminescent tag resides. The fluorescent tag enables detection with microscopy or in fixed tissues. The bioluminescent tag, such as nanoluc luciferase or -to a lesser extent firefly luciferase-enables very high discrimination of EVs in the cells or media or biofluids derived from such cells that express the construct. The affinity tags, such as Flag-tag or HA-tag, enable selection of construct-expressing cells with affinity columns or construct-expressing EV selection with affinity Dynabeads (sigma) or affinity resin (sigma). The construct has been shown to allow EV selection from specific cell populations in vivo in both dissociated tissues and biofluids, and to discriminate them from EVs produced by other cells that don't contain the construct. For example, glioma cells or hNPCs (human neuronal progenitor cells) implanted in the mouse brain and expressing the construct release reporter-containing EVs. Rare tumor-derived EVs are detectable in blood, liver, lung, etc. by use of the affinity-tag and the luminescent protein. Similarly, when injected in a mouse, reporter-expressing EVs are trackable with their affinity/bioluminescent tag in blood, liver, lung, heart etc. in vivo and postmortem in harvested organs. Release or recovery of construct expressing EVs or construct expressing cells from beads is possible with high affinity peptides such as Flag-tag peptide or HA-peptide.

In some embodiments, an interchangeable cassette in the construct expressing the CD63 construct can be used to introduce sequences complementary to gRNA targeting sequences. This cassette contains a stop codon and impedes expression of the affinity, bioluminescent, and fluorescent tag. As shown herein, only if the Cas9 and a specific sgRNA to the gRNA targeting sequence is functional in the recipient cell, the stop codon is altered and only if the construct is correctly reassembled post-gene-editing, will the cells become bioluminescent. Extra or fewer nucleotides can optionally be introduced in the sgRNA sequence to select for cells that implemented frameshifts post-gene editing. Moreover, by using both firefly and nanoluc luciferase constructs it is possible to perform multiplexing assays as both luciferases can be discriminated from each other in a single assay. Similarly, antibodies against the incorporated affinity tags can immobilize (for example, printed antibodies on a microfluidic chip) or enable detection (detection antibody with fluorescent dye) of the EVs, without the need for compromising the EV membrane layer.

When both reporter and sgRNA are oriented to gDNA targets, when cells are transfected with a plasmid that encodes both the reporter and the sgRNA (the gene editing enzyme can be introduced as protein, mRNA and/or plasmid) the following events will occur, preferably in the following order:
1. Cells will only express the new CD63 reporter construct if all components (sgRNA/Cas9 and gDNA target) are spatially and temporally present in that cell;
2 When gene editing occurs in a cell, the reporter can only be expressed if the correct repair has occurred;
3. It is significant more likely that the gene editing event occurred not only in the reporter but also in the gDNA target and not vice versa, as the plasmid-reporter DNA target is present at higher levels than the gDNA target;
4. As soon as gene editing events occur, the reporter will lead to the release of EVs with detectable bioluminescence in the supernatant/biofluids, which is a sensitive readout;
5. Cell populations post-editing that have bioluminescence signal in the media express the reporter that underwent correct gene editing and will express the affinity tag on the extracellular side;
6. As the tag is embedded in a protein template and therefore less prone to trypsin or other protease activity, successfully edited cells can be trypsinized and run over an affinity column. Only cells that express the reporter (gene edited cells) will remain in the column the others will not interact with the column. Elution of the affinity-captured cells can be performed, e.g., with peptides. This provides a population enriched for only gene edited cells, which can then be cultured or used, e.g., for ex vivo gene editing therapies.
7. After a couple of cell divisions the reporter plasmid will be diluted/no longer expressed and only the gDNA changes will remain and cells can be used for downstream analysis.

In some embodiments, this whole selection process can be performed in 48 h-72 h. The sooner the immune affinity selection occurs, the lower the chances that there will be off-target gene editing events. The technology is also adaptable to preclinical animal models. In this case, delivery methods for sgRNA/gene editing enzymes can be tested in animals. For example, transgenic tumor cells can be implanted in animals or transgenic animals can be made with the reporter construct. Hereby, researchers can anticipate the time and number of correct CRISPR/Cas9 events in a living animal.

Thus, described herein is a transmembranal protein construct that can be used to detect and isolate EV-producing cells that harbor specific gene-editing events in their genome, comprising a transmembrane domain protein, such as CD63 tetraspanin, that contains a reporter gene, e.g., a fluorescent tag and/or a bioluminescent tag; an affinity tag; and a nucleotide sequence complementary to the target gRNA sequence.

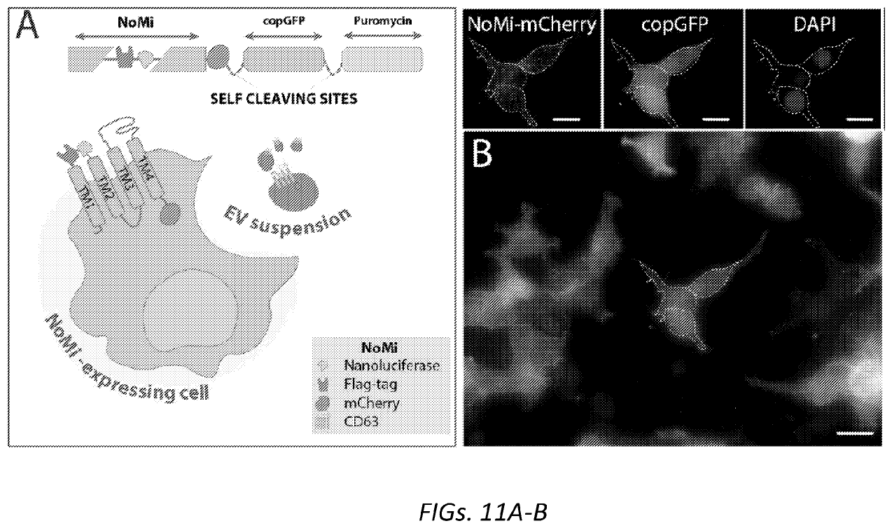

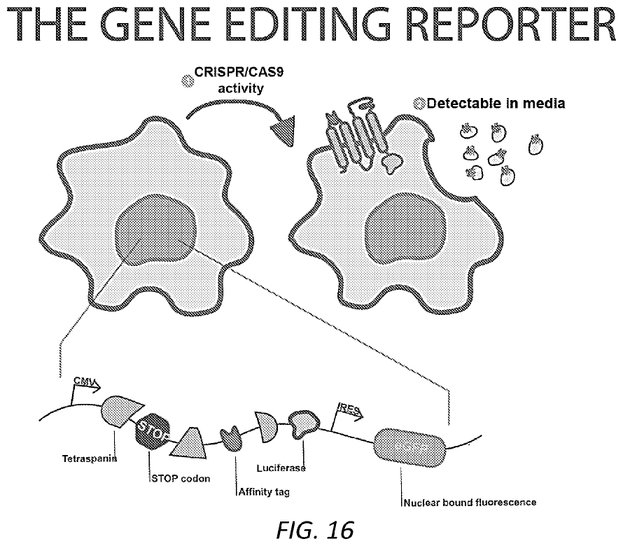

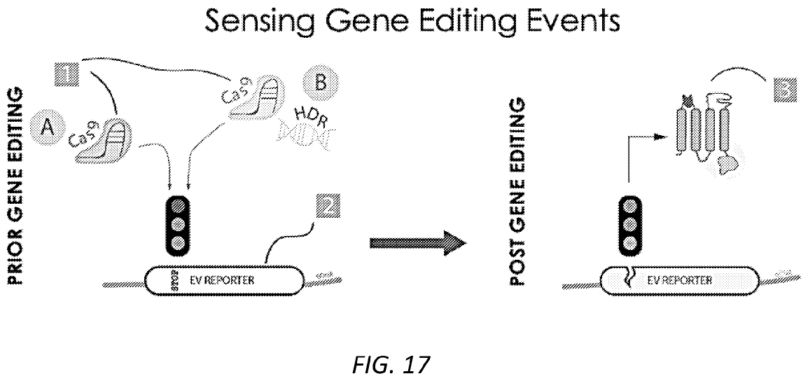

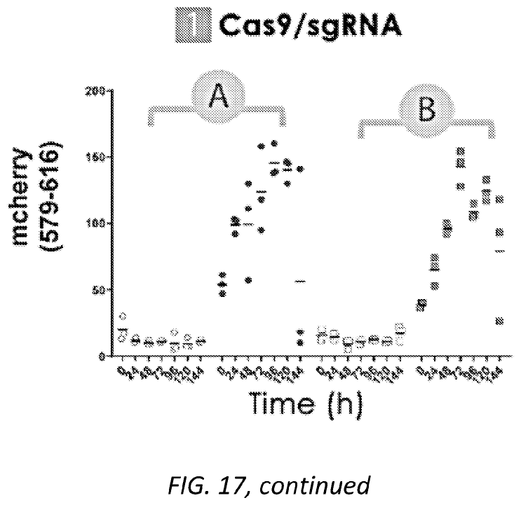

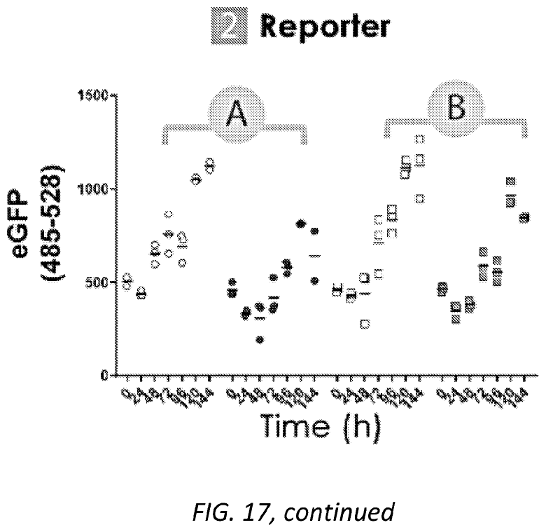

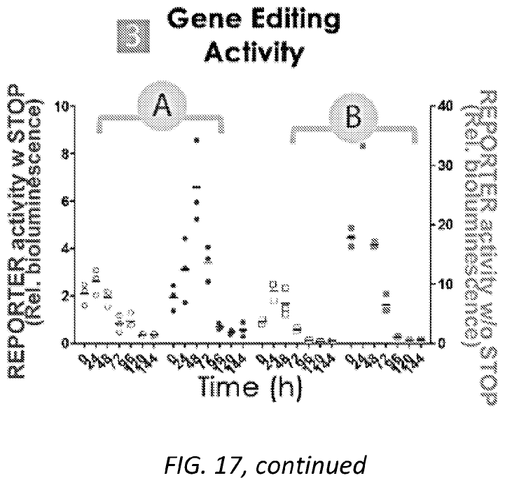

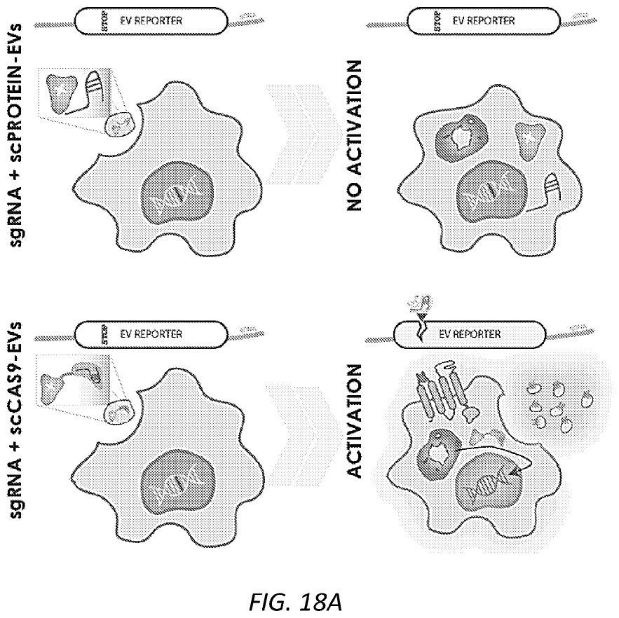

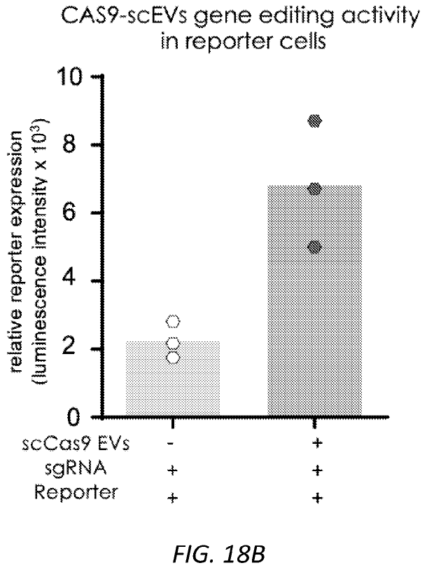

A number of transmembrane proteins can be used in the present constructs, including any of the tetraspanins, e.g., CD63, CD9, CD81. As shown in FIG. 11A and FIG. 16, preferably one of the extracellular loops of the transmembrane proteins is modified to include an affinity tag, e.g., as described herein. Alternatively, a single transmembrane domain can be linked to an extracellular domain comprising an affinity tag, and an intracellular domain comprising a reporter gene.

In addition, the sequence encoding the reporter construct includes a sequence that is identical or complementary to a gRNA targeting sequence in the genome of a cell, wherein the gene editing target harbors a variation, e.g., a pathological variation, e.g., a stop codon, e.g., a premature stop codon, that results in a pathology, e.g., a loss of expression or a loss or gain of function. Multiple guide sequences targeting the EV construct sequence can also be used. For example, in some embodiments two gRNAs can be used to cut out a sequence from the extracellular loop. That sequence can have a stop codon or a poly A sequence in-between the 2 gRNAs targets and interrupts construct translation. In that case you don't need a frame shift or stop codon, all guide RNA sequences would work and only when the two gRNAs cut simultaneously would the construct be expressed.

The construct includes at least one reporter gene that is expressed on the inside and/or at least one reporter gene that is expressed on the outside of the cell, e.g., a fluorescent and/or bioluminescent reporter gene, and thus on the inside of EVs that are produced by the cell. Fluorescent reporter genes can include green fluorescent protein or a derivative thereof, cyan fluorescent protein (CFP), red fluorescent protein (RFP), mCherry, Tag-RFP, etc.). Useful fluorescent proteins also include mutants and spectral variants of these proteins that retain the ability to fluoresce. See e.g., Shaner et al., Nat. Biotech. 22:1567 (2004), Tag-RFP (Shaner, N. C. et al., 2008 Nature Methods, 5(6), 545-551), Other fluorescent proteins that can be used in the methods described include, but are not limited to, AcGFP, AcGFP1, AmCyan, copGFP, CyPet, dKeima-Tandem, DsRed, dsRed-Express, DsRed-Monomer, DsRed2, AmCyan1, AQ143, AsRed2, Azami Green, Azurite, BFP, Cerulean, CFP, CGFP, Citrine, dTomato, dTomato-Tandem, EBFP, EBFP2, ECFP, EGFP, Emerald, EosFP, EYFP, GFP, mBanana, mCerulean, mCFP, mCherry, mCitrine, mECFP, mEmerald, mGrape1, mGrape2, mHoneydew, Midori-Ishi Cyan, mKeima, mKO, mOrange, mOrange2, mPlum, mRaspberry, mRFP1, mRuby, mStrawberry, mTagBFP, mTangerine, mTeal, mTomato, mTurquoise, mWasabi, PhiYFP, ReAsH, Sapphire, Superfolder GFP, T- HcRed-Tandem, HcRedl, JRed, Katuska, Kusabira Orange, Kusabira Orange2, mApple, Sapphire, TagCFP, TagGFP, TagRFP, TagRFP-T, TagYFP, tdTomato, Topaz, TurboGFP, Venus, YFP, YPet, ZsGreen, and ZsYellowl, all of which are known in the art, e.g., described in the literature or otherwise commercially available. Bioluminescent proteins include aequorin, and firefly or nanoluc luciferase, and variants thereof (see, e.g., Rowe et al., Anal Chem. 2009 Nov 1; 81(21): 8662-8668; Wang et al., ACS Chem. Neurosci. 2018, 9, 4, 639-650).

Also provided methods that can include expressing the reporter construct in cells, along with a CRISPR based nuclease and guide RNA, e.g., by contacting the cells with a vector or vectors comprising sequences encoding the reporter construct, nuclease, and gRNA, or a vector or vectors comprising sequences encoding the reporter construct and nuclease/gRNA RNP, or a vector or vectors comprising sequences encoding the reporter construct and gRNA and nuclease proteins. The cells are maintained until editing can occur, and then production of EVs comprising the reporter construct is monitored, e.g., by assaying culture media (for cells or tissues in vitro) or biofluids (for cells or tissues in vivo). Affinity purification using the affinity tags can be used to isolate cells and/or EVs that comprise the reporter construct.

### EXAMPLES

The invention is further described in the following examples, which do not limit the scope of the invention described in the claims.

### Example 1. Exogenous Loading of Extracellular Vesicles, Virus-Like Particles, and Lentiviral Vectors with Supercharged Proteins

Cell membrane-based biovesicles (BVs) are important candidate drug delivery vehicles and comprise extracellular vesicles, virus-like particles, and lentiviral vectors.

Here, we introduce a non-enzymatic assembly of purified BVs, supercharged proteins, and plasmid DNA called pDNA-scBVs. This multicomponent vehicle results from the interaction of negative sugar moieties on BVs and supercharged proteins that contain positively charged amino acids on their surface to enhance their affinity for pDNA. pDNA-scBVs were demonstrated to mediate floxed reporter activation in culture by delivering a Cre transgene. We introduced pDNA-scBVs containing both a CRE-encoding plasmid and a BV-packaged floxed reporter into the brains of Ai9 mice. Successful delivery of both payloads by pDNA-scBVs was confirmed with reporter signal in the striatal brain region.

Overall, we developed a more efficient method to load isolated BVs with cargo that functionally modified recipient cells. Augmenting the natural properties of BVs opens avenues for adoptive extracellular interventions using therapeutic loaded cargo.

### Materials and Methods

CELL CULTURE. Human embryonic kidney 293 (HEK293T), GL261 cells and HeLa cells were obtained from the American Type Culture Collection and were cultured at 37° C. in a 5% CO2 humidified incubator. Culture media was comprised of Dulbecco's modified essential medium (DMEM) with L-glutamine (Corning) supplemented with penicillin (100 units/ml), streptomycin (100 mg/ml) (P/S) (Corning) and 10% fetal bovine serum (FBS) (Gemini Bioproducts). Stock cells were passaged 2-3 times/week with 1:4 split ratio and used within 8 passages. Cells were monthly tested for mycoplasma contamination (Mycoplasma PCR Detection Kit, abm G238) and found negative. Cells grown for EV isolation were cultured in media supplemented with 5% EV-depleted FBS (FBS was depleted of EVs by 16 hrs centrifugation at 160,000×g).

EV AND BIOVESICLE ISOLATION FROM CELLS WITH SIZE EXCLUSION CHROMATOGRAPHY (qEV) COLUMN. EVs isolated from thirty ml of conditioned medium were collected from cells cultured at 70% confluency in two 100 mm plates after 72 h (seeding density 2.2×10⁶ cells/plate). The conditioned media was centrifuged at 300×g for 10 min to remove intact cells, dead cells and cell debris. The medium was then concentrated using a centrifugal concentrator with a 100,000 molecular-weight cutoff (Amicon®Ultra-15 Centrifugal filters), yielding about 0.5 ml concentrate (two spins of 15 ml at 6000×g for 10 min). This concentrate was resolved by passing through IZON qEV original size exclusion columns (SEC) followed by 15 ml of double filtered (0.2 μm) PBS. Five-hundred-microliter fractions were collected. High particle/low protein fractions (from 7 to 11) were pooled and concentrated using Amicon®Ultra-0.5 Centrifugal filters to a final volume of 200 μL at 10,000g for 30 min. The typical yield of an EV isolation was approximately 7.1×10⁷±3.2×10⁷ particles/ml. This method was adapted to isolate EVs, LVVs (transgene plasmid, psPAX2 (Addgene #12260) and pMD2.G (#12259), VSV G-VLPs (pMD2.G (Addgene #12259), and GAG-VLPs (psPAX2 (Addgene #12260)) before being exposed to scProteins. LVVs purified from media of 2.5 million HEK293Tcells transfected with psPAX2 (Addgene #12260) and pMD2.G (Addgene #12259) were isolated with SEC.

LOADING OF BIOVESICLES WITH scPROTEIN. 5×10¹² concentrated EVs (based on Nanosight measurement) were loaded in 50 μl with 283 nM recombinant scProtein and incubated for 15-45 min with gentle agitation on a HulaMixer™ at room temperature. Then 50 μl Ni-NTA agarose resin (Qiagen) or NEBExpress Ni-NTA Magnetic Beads (NEB) in PBS were added, incubated for 15-30 min to 24 h on a HulaMixer™ and compared based on fluorescence to a control of scProtein and agarose resin (Qiagen) without EV suspension. After centrifugation for 30 sec at 14,000×g, the supernatant was collected leaving the resin with the bound scProtein that was not associated with the EVs. Fluorescence in suspension was visually inspected using a UV lamp with black background and quantified with a microplate reader (Synergy H1 Hybrid Multi-Mode Reader, BioTek) at an excitation wavelength of 485 nm for GFP or 433 nm for mCerulean3. Similarly, +scProtein solution was exposed to liposomes (100-200 nm vesicles based on Nanosight) at a concentration of 6.29×10⁸ particles/ml that were kindly provided by Dr. Van Solinge. The liposomes were diluted to match our EV sample with 7.5×10⁷±1.2×10⁷ particles/ml (based on Nanosight). The liposomes used were negatively charged DPPC-PEG(2000)-DSPE-cholesterol liposomes, which have been characterized in depth by Deshantri et al. 2019⁴⁹. This method was also adapted for loading LVVs, GAG-VLPs, VSV G-VLPs GAG/VSV G-VLPs. The carrier LVV was concentrated with spin filters (see above) and 30 μl of this suspension was loaded with both plasmid (1 μg) and scProtein (2.2 mol). The total solution of 90 μl was incubated with gentle agitation on a HulaMixer™ at 4° C. overnight to generate scLVV. Then 90 μl samples were added to cells at a density of 50,000 cells in each well of a 12-well plate. Genomic viral RNA (vRNA)-carrying EVs, GAG-VLPs and VSV G-VLPs were generated similarly to scLVV, but were generated either from cells expressing psPAX2 encoding pol and GAG (GAG-VLPs), or pMD2.G encoding for VSV G (VSV G-VLPs).

scEV CHARACTERIZATION WITH EXOVIEW. A sample of EVs purified from HEK293T cells (see above) was concentrated with spin filter columns (Milipore 30 kDa) to a final volume of 30 μl. These EVs were either loaded with 20 μl 283 nM scProtein (see procedure above) or remained unloaded at 4° C. overnight. According to the guidelines provided by NanoView Biosciences (USA), the samples were incubated on the ExoView Tetraspanin Chip for 16 h at room temperature. After washing the chips three times in 1 ml PBS for 3 min, they were incubated with ExoView Tetraspanin labeling antibodies (1:500 in PBST) with 2% BSA for 2 h. The chips were rinsed with PBS and then imaged with the ExoView R100 reader. Procedure and initial analysis were performed by the ExoView representative.

scPROTEIN PRODUCTION. One Shot® RBL21 Star™ (DE3; Invitrogen) bacteria were transformed with plasmids encoding the scProtein of interest and plated on LB agar plates with appropriate antibiotic (kanamycin 50 μg/mL, Sigma). After overnight incubation at 37° C., a medium sized, isolated colony was picked and inoculated into a 5 mL overnight seed culture of LB media. The seed culture was diluted 1:20 and grown in 2×YT media (Sigma) supplemented with 0.2 um filtered 40 mM MgSO4 (Sigma), 2.5 mM KCI (Sigma), and 20 mM glucose (Sigma) until obtaining OD600 ˜0.1-0.3 and then, for protein expression, the bacteria were induced by adding 0.5 mM isopropyl β-d-1-thiogalactopyranoside (IPTG; Sigma) and incubated overnight at 37° C. Cultures were harvested by centrifugation at 3000×g for 10 min and pellets stored at −80° C. or processed immediately.

BIO-BEADS PULL-DOWN OF scEVs. Bio-Beads SM-2 resin (Biorad) were suspended in PBS to 0.2 g/ml. Subsequently, 50 μl of the Bio-Bead solution was added to a 500 μl scEV suspension generated from a mixture of 7.5×10⁷+/−1.2×10⁷ EV particles/ml and 10 pmol of +scProtein or a scProtein solution without EVs. The samples were incubated overnight at 4° C. degrees with gentle agitation on a HulaMixer™. As a control for Bio-Bead pull-down, a 10% Triton X-100 solution was incubated under the same conditions. The following day, the Bio-Beads were spun down (6000×g for 1 min) and resuspended in 50 μl PBS. 50 μl of the supernatant and the Bio-Bead solution were measured with a Synergy H1 Hybrid Multi-Mode Reader (Biotek).

REMOVAL OF N-GLYCOSYLATED MOIETIES. 7.5×10⁶ EVs in 100 μl solution prior or after scProtein loading were incubated with 10 μl Heparinase I/II/III seperately from Flavobacterium heparinum (Sigma) or with PNGase F (New England Biolabs). For optimal enzyme activity, a buffer solution was added provided by the supplier and the samples were incubated for 24 h at 37° C. or 25° C., respectively, in a HulaMixer Sample Mixer (Invitrogen). Ni-NTA affinity resin was added and pelleted (16,000 g for 5 min) to exclude unloaded scProtein. scProtein fluorescence was measured after incubation for 15-30 min to 24 h in Ni-NTA supernatant and Ni-NTA resin pellet.

CD63 IMMUNOAFFINITY BEADS FOR scEV CHARACTERIZATION. 20 μl of antiCD63 beads (invitrogen) were washed twice with 0.1% BSA inPBS and provided with 100 μl suspensions of HEK293T scEVs, HEK293T EVs, scProteins, HEK293T Bodipy TR labelled scEVs, or HEK293T EVs. The samples were incubated overnight at 4° C. with gentle agitation on a HulaMixer™. Beads were washed twice with 0.1% BSA/PBS and when needed incubated with antiCD63-APC (MEM-259, Invitrogen) in a 100 μl volume with 0.1% BSA/PBS for 1 h at 4° C. with gentle agitation. Samples were washed twice by centrifugation (300 g for 15 min) and dissolved in 250 μl 0.1% BSA/PBS for flow cytometry measurement.

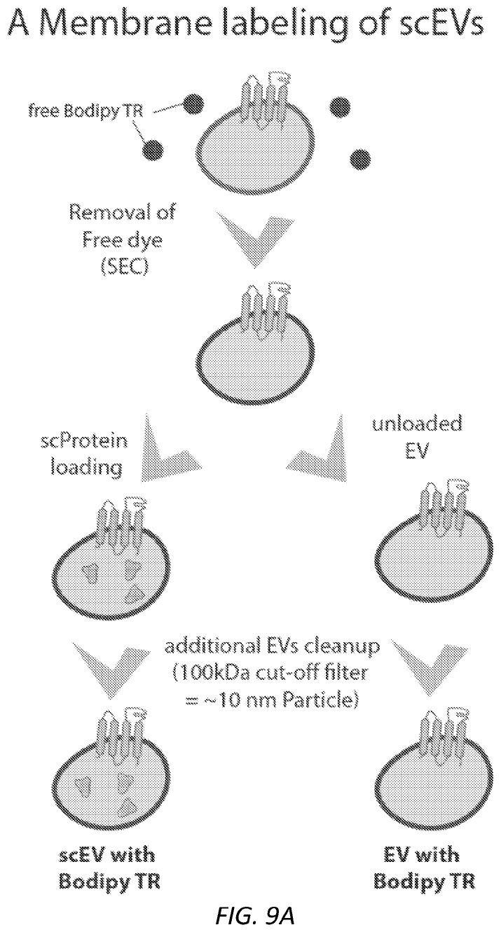

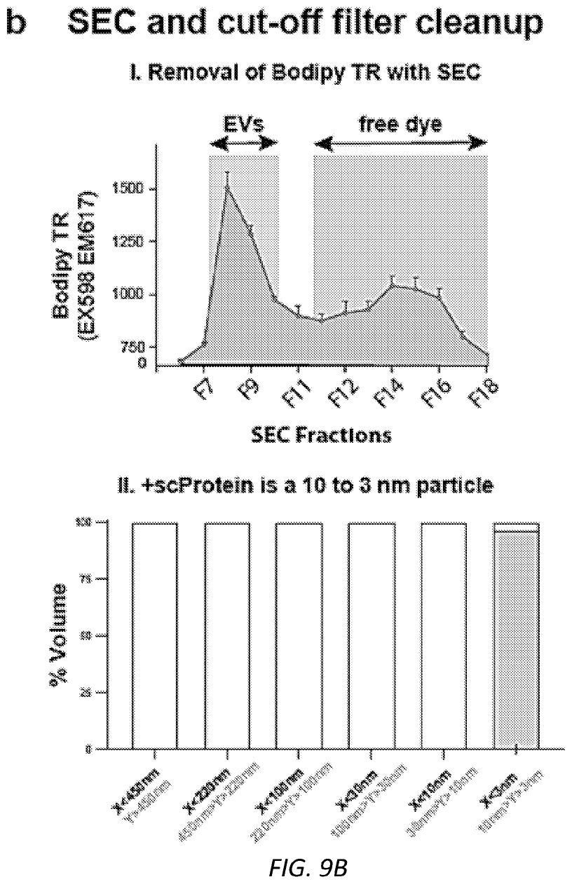

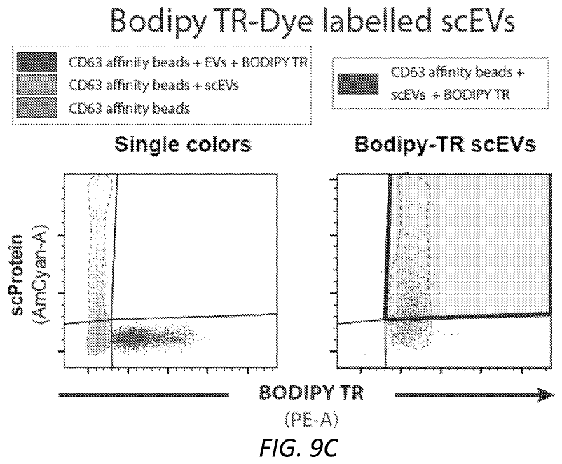

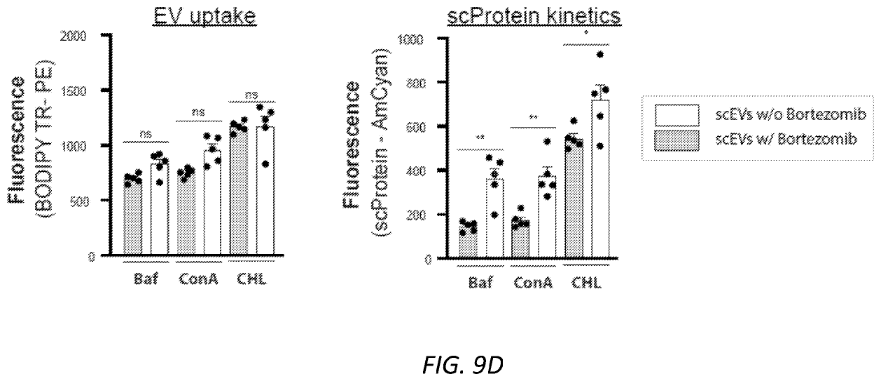

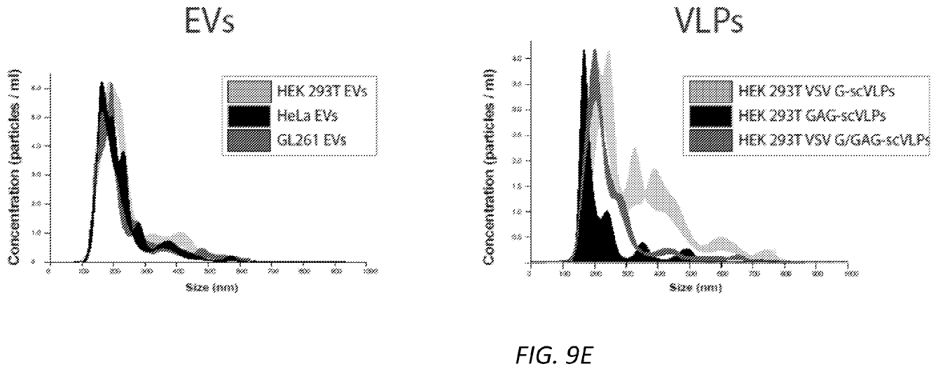

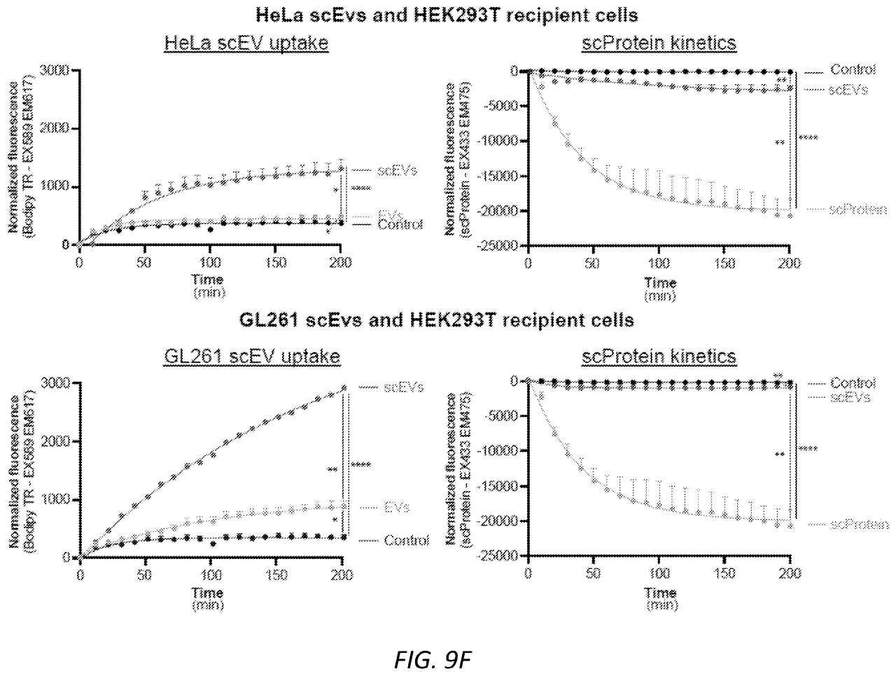

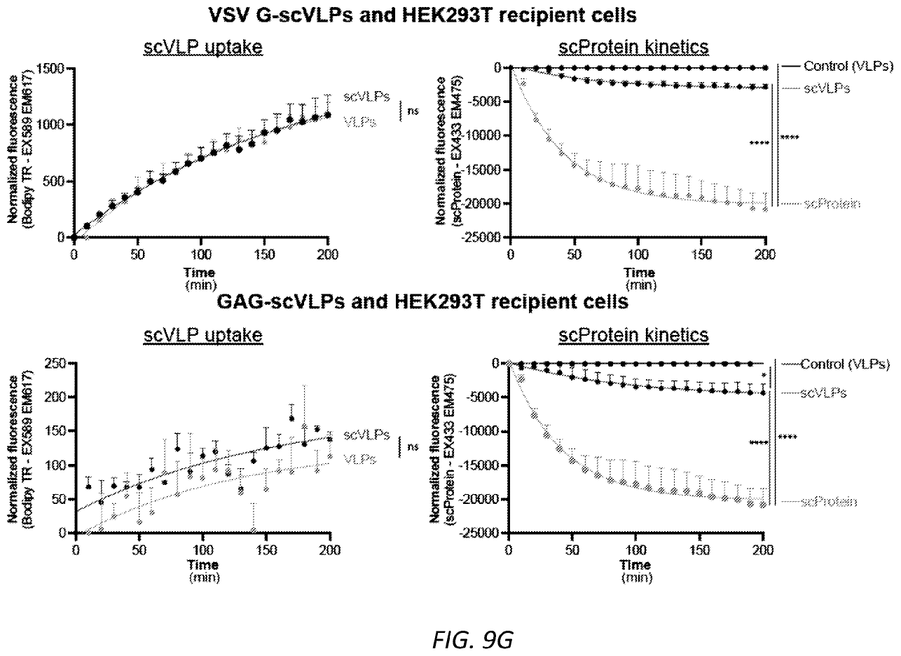

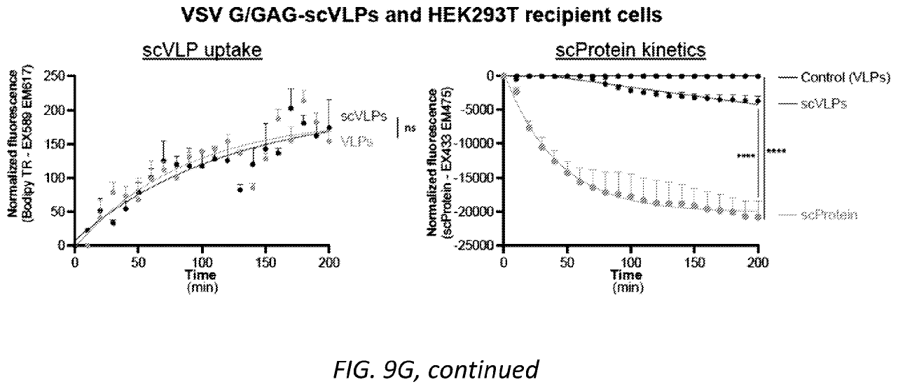

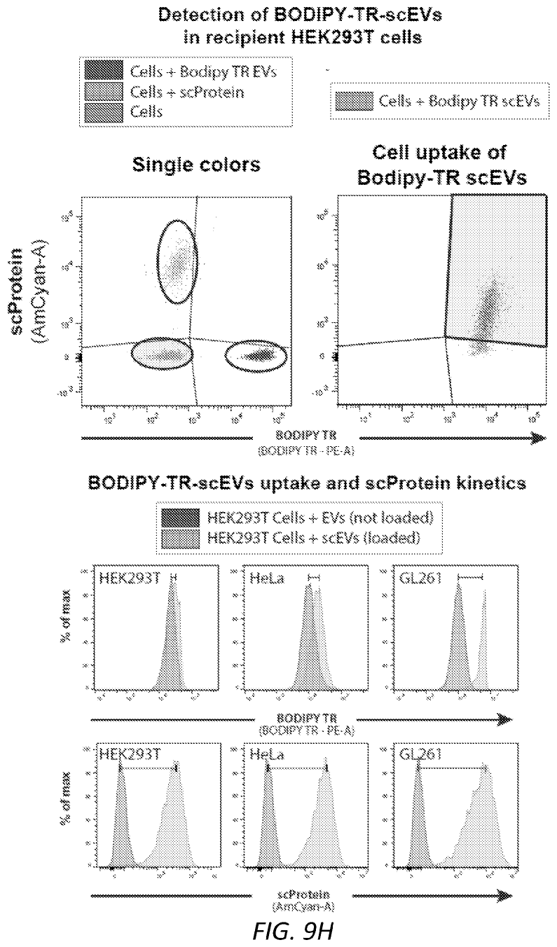

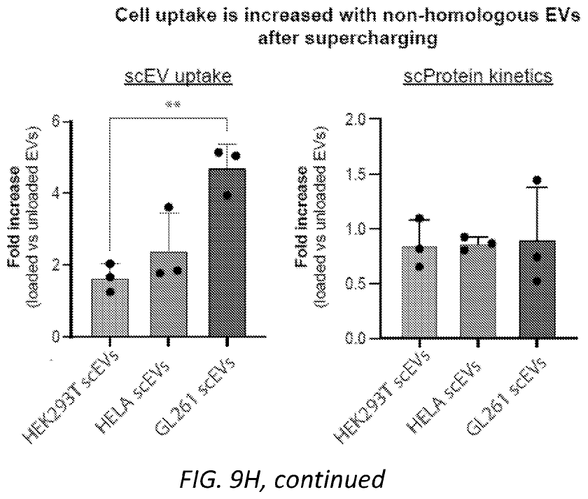

LABELING OF scEVs AND UPTAKE. 1.8-2.0×10⁷ EVs from HEK293T, HeLa, or GL261 cells in 30 μl PBS each were labelled with 10 μM Bodipy™TR-ceramide (ThermoFisher) for 1 h on a HulaMixer at 37° C. (FIG. 9-A). To remove excess dye, EVs were isolated by SEC. Samples were divided such that one part was supercharged with 50 pmol scProtein, as described above, while the other part was incubated in the same volume of PBS. Lastly, the samples were applied to a 10 nm (˜100 kDa) cut-off filter (milipore) as an additional step to eliminate free scProtein (30.1 kDa=˜3 nm). To monitor EV and scEV uptake, HEK293T cells (10,000 cells per well) were seeded in 96-well plates (Falcon) in optimem culture medium (Gibco) overnight in serum-free media. Images were acquired every 10 min for 200 min using a Synergy H1 Hybrid Multi-Mode Reader (BioTek). The average fluorescent intensity of scProtein and Bodipy TR was normalized to the starting value. Flow cytometry experiments of scEV uptake after 24 h were performed, as described below, separately from 200 min monitoring experiments.

For inhibitor treatment before scEV uptake, HEK293T cells (50,000 cells per well) were seeded in 24-well culture plates (Falcon) for 24 h in DMEM with serum. The cells were then pre-incubated with the inhibitors in Optimem without serum. The following inhibitors were used: 200 nM bafilomycin A1 (Sigma-Aldrich), 200 nM concanamycin A (Sigma-Aldrich), 100 μChloroquine (Sigma-Aldrich) or 5 μBortezomib (Millipore). The labelled EVs (10⁷-10⁸ particles) were added each well for 24 h in the presence of the indicated inhibitors. Following incubation, cells were trypsinized and analyzed based on scProtein and Bodipy TR fluorescence on a Beckman SORP 5 Laser BD Fortessa Flow Cytometer in the MGH core facility.

For uptake of different EV sizes, 3×10⁸ per 0.5 ml BODIPY-TR labelled scEVs were serial filtered through 450 nm (Costar), 220 nm (Costar), 100 nm (Costar), 300 kDa (˜30 nm, Millipore), 100 kDa (˜10 nm, Millipore), and 30 kDa (˜3 nm, Millipore) cut-off filters. Images were acquired every 10 min for 200 min using a Synergy H1 Hybrid Multi-Mode Reader (BioTek). The average fluorescent intensity of scProtein and Bodipy TR was normalized to the starting value.

NUCLEUS ISOLATION POST-scEV EXPOSURE OF HEK293T CELLS. 10⁷-10⁸ scEVs and EVs derived from HEK293T, HeLa, and GL261 cells were incubated with 50,000 HEK293T cells per 24-well. After 4 days of incubation, cells were trypsinized and washed with PBS. Cell fractionation kit (Abcam, 109718) was used to extract cytosolic, mitochondrial, and nuclear proteins. scProtein fluorescence was measured with a Synergy H1 Hybrid Multi-Mode Reader (BioTek).

ASSEMBLY OF scEVs WITH PLASMID DNA (pDNA). 10⁷-10⁸ HEK293T EVs, GL261 EVs, HeLa EVs, VSV G-VLPs, GAG-VLPs, VSV G/GAG-VLPs and LVVs were incubated with 50-100 pmol scProtein and 1 μg pDNA overnight at 4° C. Binding of pDNA to complexes was verified on 1% agarose gels. To test the stability of assembly, 2 units of TURBO Dnase I (Invitrogen) was added at 37° C. for 15 min and compared to pDNA/scProtein or pDNA with and without DNase treatment. pDNA used for this experiment has been summarized in Table 1.

TABLE 1
Plasmids used in this manuscript:

| Transgene | Source |
|---|---|
| sfGFP | Addgene #85492 |
| −scGFP | Addgene #62936 |
| +scGFP | Addgene #62937 |
| +scmCerulean3 | Herein |
| +scmCerulean3-NLS | Herein |
| CD63-nanoluc-flag-mCardinal | Herein |
| FLEx-GFP-floxed nanoluc | Herein |
| mCherry LVV plasmid | Addgene #78534 |
| mCre plasmid | Herein |
| bCre plasmid | Herein |

BIOLUMINESCENT AND FLUORESCENT ASSAYS. Recipient cells were trypsinized and seeded in 24-well plates (50,000 cells/well) in 500 μl complete DMEM media. After 24 h, 100 μl of the pDNA-scEV suspension was added to the cultured cells. Each day nanoluciferase³⁷was monitored by removing 50 to 100 μl of media. Nanoluciferase expression was analyzed with the addition of furimazine (Nano-Glo®Luciferase, Promega) diluted in 1×PBS in a range from 1:250 to 1:500. Samples were incubated with the reagent for at least 3 min prior to reading on Synergy H1 Hybrid Multi-Mode Reader (BioTek). For luminescent readings, samples were loaded into white 96-well culture plates (Lumitrac 200). For fluorescent readings, the samples were loaded into black 96-well culture plates (10,000 cells/well). Each sample was loaded in triplicate with a volume of 100 μl in each well. Biovesicles were loaded with pDNA as listed above. Coronal tissue samples from mouse brain corresponding to 150 μm thick sections were homogeneized in 500 μl Nano-Glo® Luciferase Assay Buffer. Bioluminescence were analysed by adding 100 μl sample and 100 μl 1:250 furimazine (Nano-Glo® Luciferase, Promega) in 1×PBS. The excitation laser was shut off, and the emitted light was measured at two different gains: 135 and 200.

ANIMALS. All animal experiments were conducted under the oversight of the MGB Institution Animal Care and Use Committee. Ai9 mice³¹ were maintained with unlimited access to water and food under a 12-hour light/dark cycle. Male and female Ai9 mice ranging from 8-10 weeks in age were randomly assigned to experimental groups (N=5 treated, N=4 control).

STEREOTAXIC INJECTIONS INTO STRIATUM. Mice were all stereotactically injected into the right striatum (coordinates: anteroposterior: +0.6 mm, lateral: ±1.8 mm, ventral: −3.3 mm) with nanobiologicals in a final volume of 4 μl containing scProtein, LVV encoding FLEx-reporter and/or mammalian CRE plasmid. Control animals were injected with 4 μl containing scProtein, LVV encoding FLEx-reporter and bacterial CRE plasmid, all at an infusion rate of 0.25 mL/min using a 10 mL Hamilton syringe. Five min after the infusion was completed, the needle was retracted 0.3 mm and allowed to remain in place for an additional 3 min prior to its complete removal from the mouse brain³⁵.

MOUSE TISSUE PREPARATION FOR IMMUNOHISTOCHEMISTRY, BIOLUMINESCENCE AND RT-PCR. Mice were sacrificed with a 100-200 μl bolus of ketamine (17.5 mg/ml) and Xylazine (2.5 mg/ml) intraperitoneally followed by an intracardiac perfusion with 50 ml PBS. Brains were collected and frozen at −80° C. Coronal sections of the striatum at 16 μm thickness were obtained using a cryostat (LEICA CM3050S, Leica Microsystems). Sections were alternately collected for immunohistochemistry, bioluminescence and RT-PCR.

STATISTICAL ANALYSIS AND REPRODUCIBILITY. Data were analyzed using GraphPad Prism 9, version 9.1.0 (GraphPad Software Inc., La Jolla, CA). All statistical tests were two-sided and a p-value of less than 0.05 was considered statistically significant. Data were presented as the mean ±S.E.M. The statistical tests used are indicated in the figure legends. Multiple comparisons of significance between groups were performed using the Tukey procedure for ANOVA or Dunn's multiple comparison test for Kruskal-Wallis, as indicated in the corresponding figure legends. The statistical analyses for uptake of EVs was modeled with non-linear regression using a one-phase association or one phase-decay equation. Graphical illustrations in figures were done in Adobe Illustrator 26.0.2.

TRANSMISSION ELECTRON MICROSCOPY (TEM). A 20 μl HEK293T EV sample was directly applied on the grid. After 1 min absorbance onto the grid, the excess liquid is blotted off the film surface using a filter paper (Whatman). The grid is floated on a small drop (˜5 μl) of staining solution (0.75% uranyl formate, 1% uranyl acetate or 1-2% PTA). After 20 seconds, the excess stain is blotted off and the sample is air dried briefly before it's examined in the TEM. Images were captured at the HMS electron microscopy core facility using Tecnai G2 Spirit Bio TWIN transmission electron microscope.

BULK BIOVESICLE NUMBER ESTIMATION WITH NANOPARTICLE TRACKING ANALYSIS. Number of BVs diluted in PBS was assayed using Nanoparticle Tracking Analysis Version 2.2 Build 0375 instrument (Nano Sight). Particles were measured for 60 s and the number of particles (30-800 nm) was determined using NTA Software 2.2. Samples were diluted 1:1000 in PBS prior to analysis. The following photographic conditions were used: frames processed (1498 of 1498 or 1499 of 1499); frames per second (24.97 or 24.98 f/s); calibration (190 nm/pixel); and detection threshold (6 or 7 multi).

PLASMID CONSTRUCTION. For production of bacteria expressing recombinant recombinant proteins construct were cloned into the pET28a vector (Addgene #85492). The plasmid was restricted with Ncol and Xhol and used as backbone for Gibson assembly (New England Biolabs) to insert the gBlock (IDT) expression cassettes of scProteins (+scGFP, −scGFP, GFP, +scCER, +NLS−scCER, see amino acid sequences in supplementary data). Each assembly reaction contained approximately 100 ng insert and 50 ng expression vector and was incubated at 50° C. for 30 min-4 hrs following the manufacturer's protocol. After the assembly reaction, the reaction mix was transformed into NEB 5-alpha competent E. coli strain (New England Biolabs) or One Shot®TOP10 Competent Cells (ThermoFisher). After overnight growth at 37° C. on Kanamycin 50 μg/mL (Sigma) containing agar plates [10 ml Bacto agar (Sigma) with LB medium in a 60 mm dish], single colonies were selected and grown with Kanamycin 50 μg/mL (Sigma) containing LB broth (Sigma). Single colony suspensions containing respective plasmid with insert were extracted using a QIAprep Spin Miniprep Kit (Qiagen). To confirm correct insertion, a restriction digest was performed, and fragments electrophoresed in a 1.5% agarose gel and stained with arose gel and stained with GelRed (Biotium). Images were acquired under UV light using Azure Biosystems c300 Image. When correct profiles were detected, complete plasmid sequencing using next-generation sequencing technology (MGH CCIB DNA Core) was performed to validate plasmid integrity. Vectors generated following this procedure and used for this manuscript are summarized in Table 1.

ScPROTEIN PURIFICATION. The protein purification protocol is an adaptation of Thompson et al., 2008. Frozen pellets were thawed and resuspended in 50 mL PBS with 2 M NaCl, 20 mM imidazole, pH 7.5, with one tablet of EDTA-free Complete Protease Inhibitor (Roche). The resuspended pellets were divided into two fractions and lysed by sonication in a Sonic Dismembrator 550 (Fisher Scientific) for 5 min. Cell debris was removed by centrifugation at 4000×g for 10 min. The supernatant was transferred to a new 50 mL conical tube and 1 mL of settled Ni-NTA agarose resin (Qiagen) was added to the bacterial lysate and incubated at 4° C. for 30-45 min with a HulaMixer™. Then the supernatant and the Ni-NTA resin were transferred to a column. The packed resin was rinsed first with 20 mL PBS and 2 M NaCl, then with 15 mL of PBS, 2 M NaCl, and 20 mM imidazole, and finally eluted with 2 mL PBS, 2 M NaCl, and 500 mM imidazole. To remove the imidazole from the protein solution it was dialyzed against 1 L PBS at 4° C. for 1 hr, and subsequently dialyzed overnight with 2 L fresh PBS buffer. Proteins were quantified via fluorescence and BSA protein (Pierce™, ThermoFisher) assay. Purity and protein size was confirmed by SDS-PAGE and nitrocellulose blot transfer. Protein bands were visualized with Pierce™ Reversible Protein Stain Kit for Nitrocellulose (Thermo Scientific). Aliquots were stored at −80° C.

FLOW CYTOMETRY. Flow cytometric analysis was performed on the Beckman SORP 5 Laser BD Fortessa Flow Cytometer in the MGH core facility. Forward and side scatter signals were used to distinguish live cells from polystyrene beads. Proper gating was performed to identify positive fluorescent signals compared to non-stained or single stained controls.

LENTIVIRAL VECTOR (LVV) PRODUCTION AND CELL TRANSDUCTION. LVVs were produced in HEK293T cells with a three-plasmid system, following Addgene recommendations. Briefly, cells were seeded and 24 hrs later, transfected with psPAX2 (#12260) and pMD2.G (#12259) packaging plasmids and the transgene of interest flanked by LTRs. Six hrs after transfection, cells were rinsed with PBS and media was replaced. Lentiviral isolation was performed 72 hrs later by ultracentrifugation at 70,000×g and the pellet was re-suspended in 1% BSA in PBS36. The viral particle content was evaluated by assessing HIV-1 p24 antigen levels by ELISA (Retro Tek, Gentaur, Paris, France). Concentrated viral stocks were stored at −80° C. until use. LVVs were used for generating stable cell lines after selection by either antibiotic resistance or flow sorting. HEK293T and HELA cells were transduced 24 hrs after plating with LVVs (400 ng of P24 HIV antigen per 200,000 cells). Twenty-four hours later, the medium was replaced with DMEM media with antibiotics and FBS, and cells were cultured and expanded under standard conditions. Stable cell lines were obtained and mCherry or GFP fluorescence was monitored at every passage.

IMMUNOHISTOCHEMISTRY. Sections were fixed with 4% paraformaldehyde for 10 min and incubated with blocking solution [0.1% Triton X-100 containing 10% normal goat serum (SigmaAldrich) in PBS] and then incubated overnight at 4° C. in blocking solution with primary antibody: rabbit anti-RFP antibody (1:250Invitrogen, polyclonal). Sections were rinsed with PBS 5 times and incubated for 2 hr at room temperature with the secondary antibody: goat anti-rabbit TRITC 594 (1:1000) diluted in blocking solution. The sections were washed and mounted in VECTASHIELD® Antifade Mounting Medium (Vector Labs Cat# H-1000) on gelatin-coated slides. Immunoreactivity of mouse sections was visualized and analyzed in Keyence BZ-X810 All-in-one Fluorescence imaging microscope.

DNA AND RNA EXTRACTIONS. The DNeasy Blood & Tissue Kit (Qiagen) was used for genomic DNA extraction from cells. QIAprep Spin Miniprep Kit (Qiagen) and EndoFree MaxiPlasmid Kits (Qiagen) were used for plasmid extraction from bacteria. RNA was extracted using the miRNeasy Mini Kit (Qiagen), according to manufacturer's protocol. The RNA concentration and integrity (RIN score) were determined using the Nanodrop (ThermoFischer Scientific) and Agilent 2100 Bioanalyzer Pico-chips (Agilent Technologies), following manufacturer's protocol.

CONFOCAL MICROSCOPY. 10,000 cells were seeded on a 35 mm optical Petri dish (Thermofisher) and incubated with approx. 10\hat{ }6 scEVs for 5 days at 37° C. Prior to imaging, LysoTracker Red DND-99 (Invitrogen) was added to the media to a final working concentration of 50 nM for 1 h. The cells were washed twice with fresh media prior to imaging on a Zeiss LSM710 Laser Scanning Confocal of the MGH Cancer Center Translational Imaging Core. Of note, Lysotracker Red DND-99 was incubated under similar conditions with isolated EVs and did not stain our scEVs. To detect nuclear translocation 10,000 cells were seeded on an optical Petri dish (theromofisher) and incubated with 10\hat{ }6 scEVs for 5days at 37° C. Cells were fixed with paraformaldehyde for 15 min at RT, washed twice with PBS and incubated with a AF594-Phalloidin (Invitrogen) according to manufacturer instructions. After twice washing with PBS, Dapi was provided through VECTASHIELD® Antifade Mounting Medium (Vector Labs Cat# H-1000). ImageJ version 2.1.0/1.53c was used for analysis.

### Example 1.1 Exogenous Loading of Extracellular Vesicles with Positive Supercharged Proteins

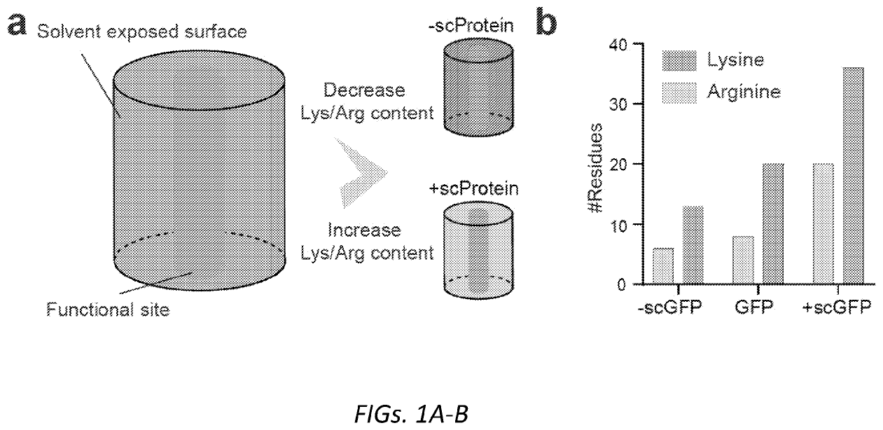

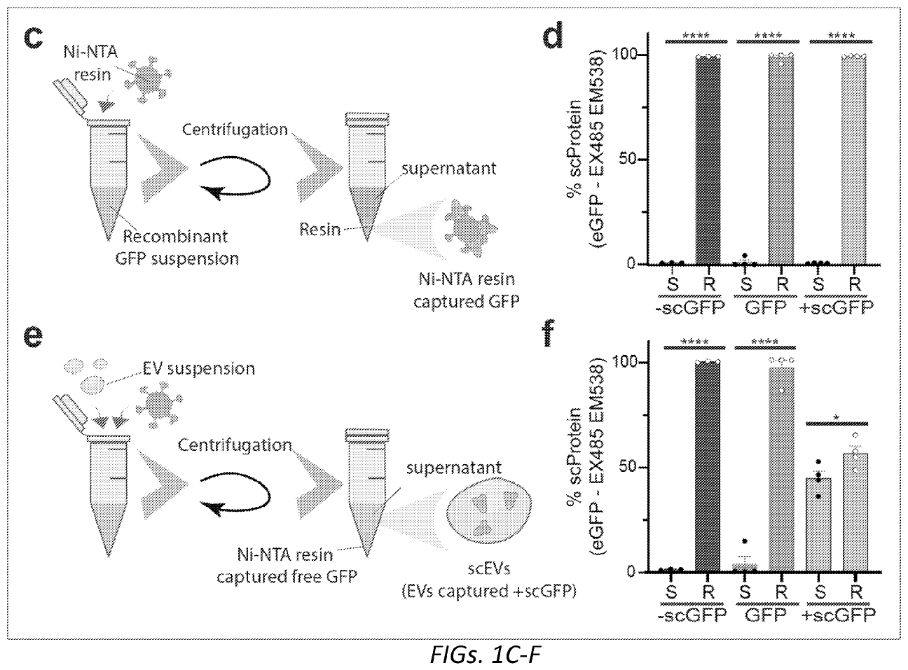

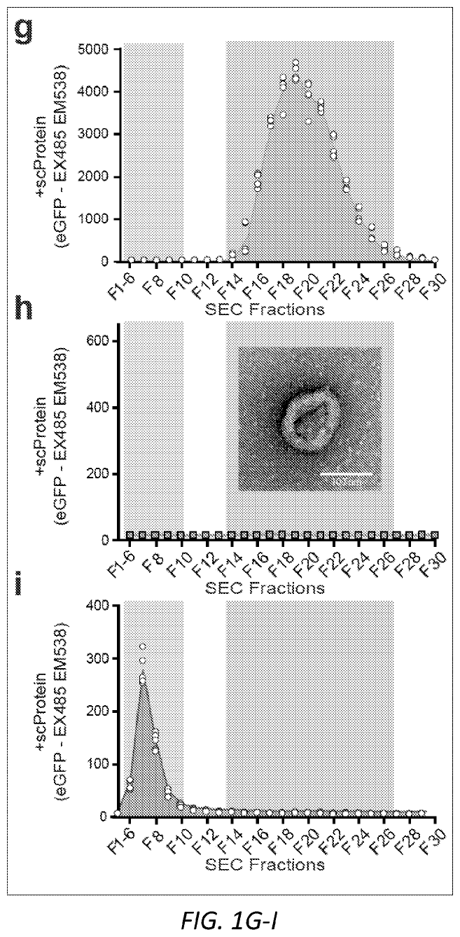

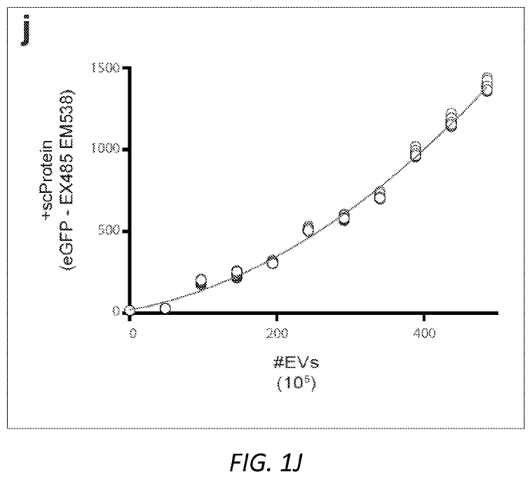

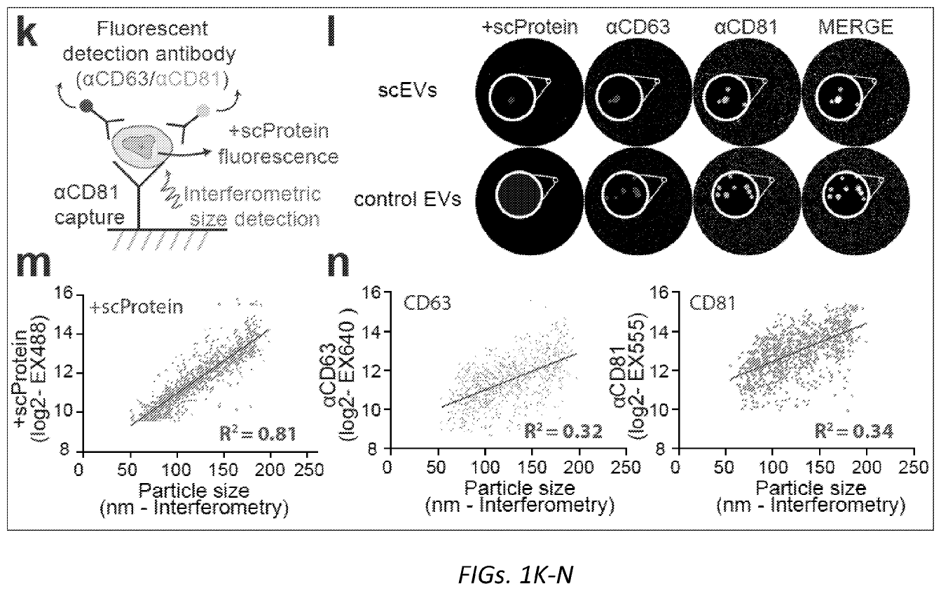

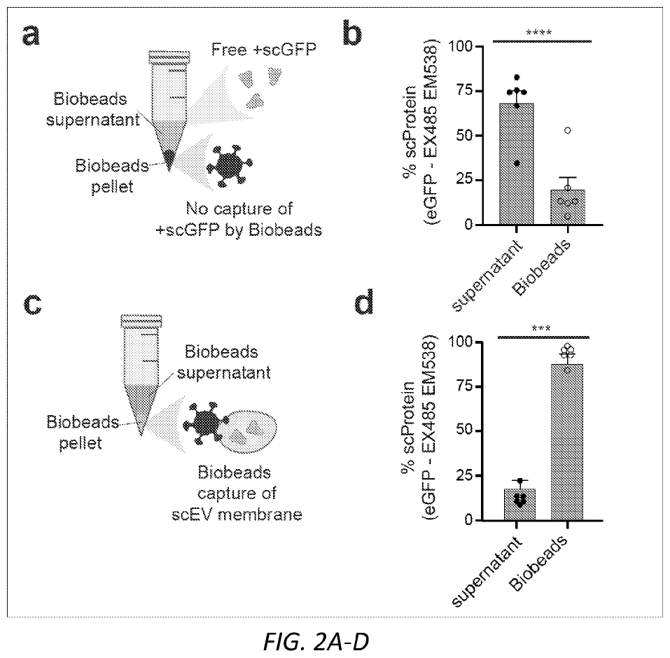

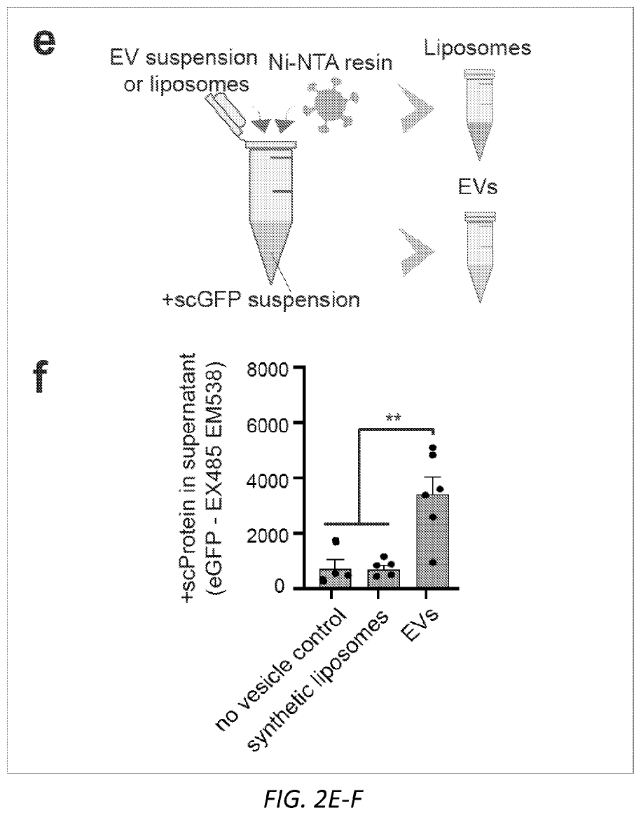

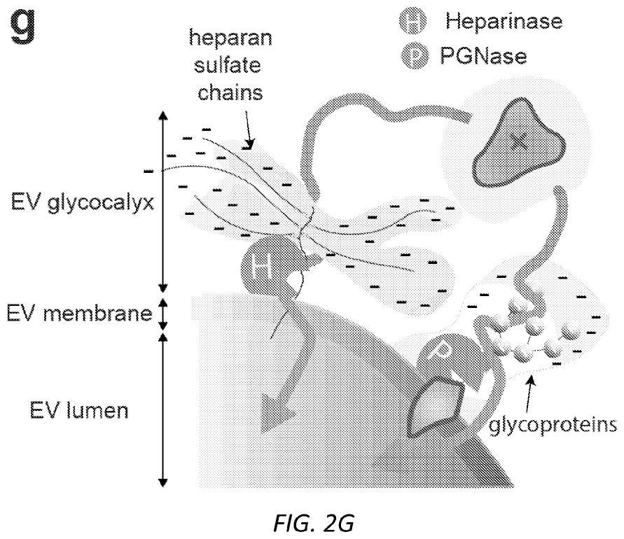

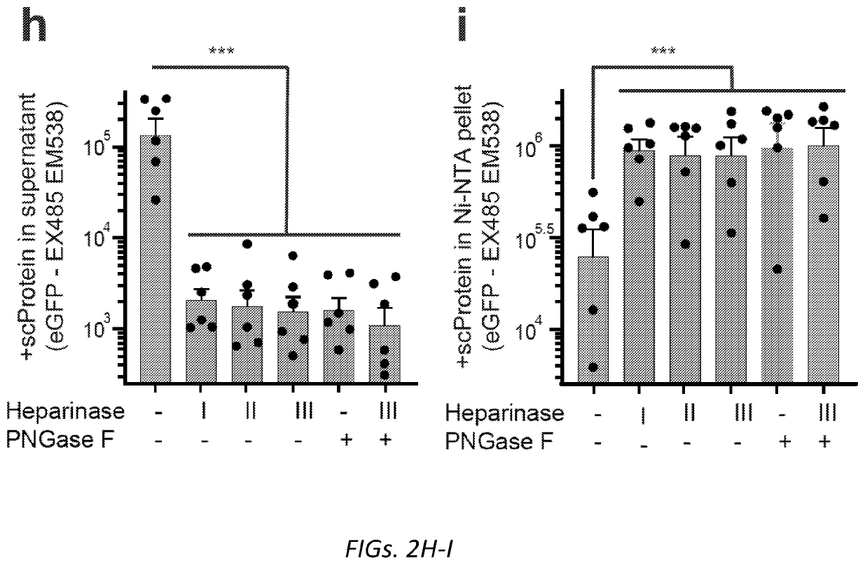

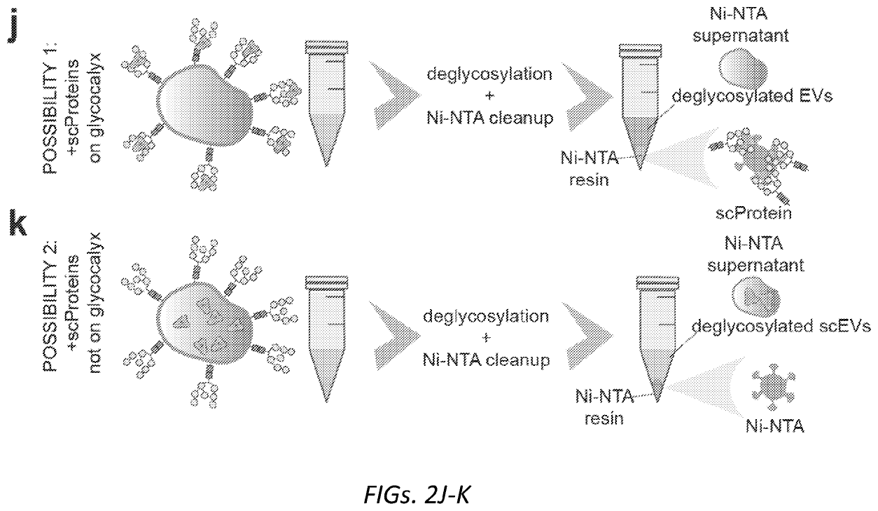

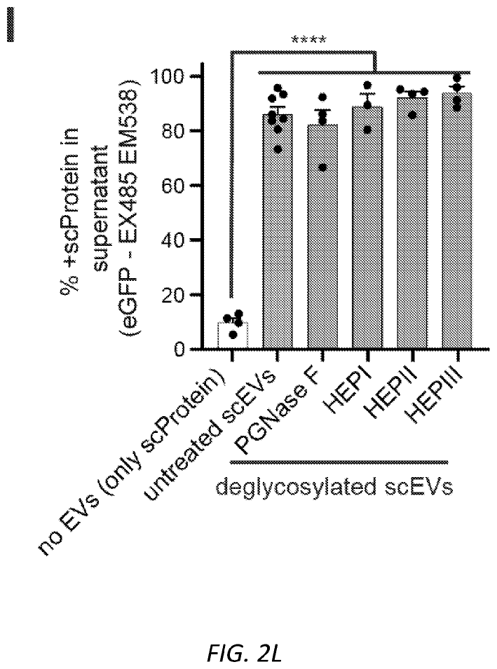

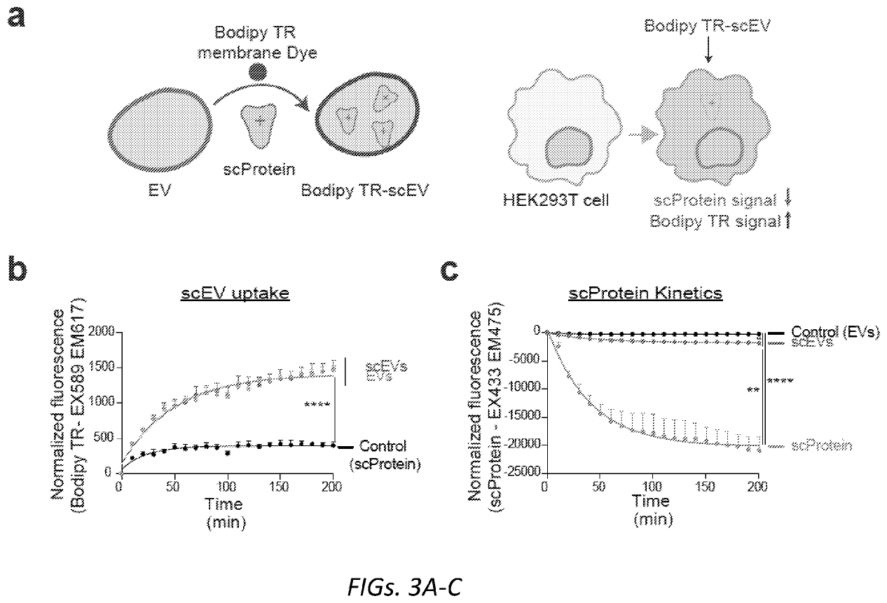

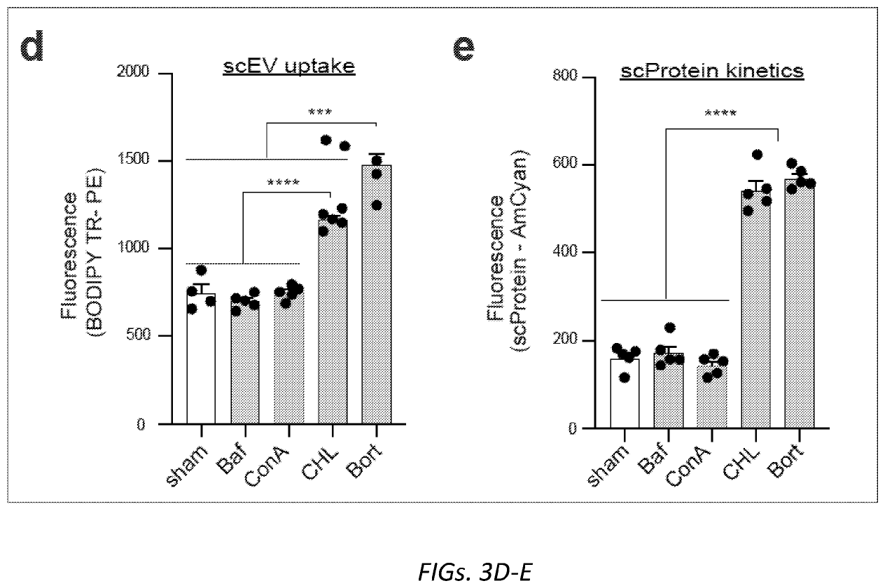

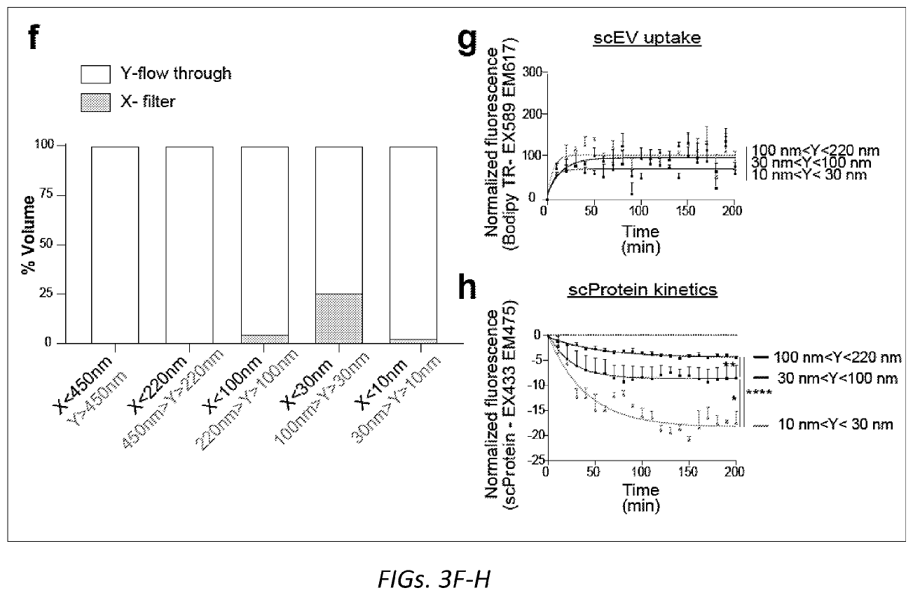

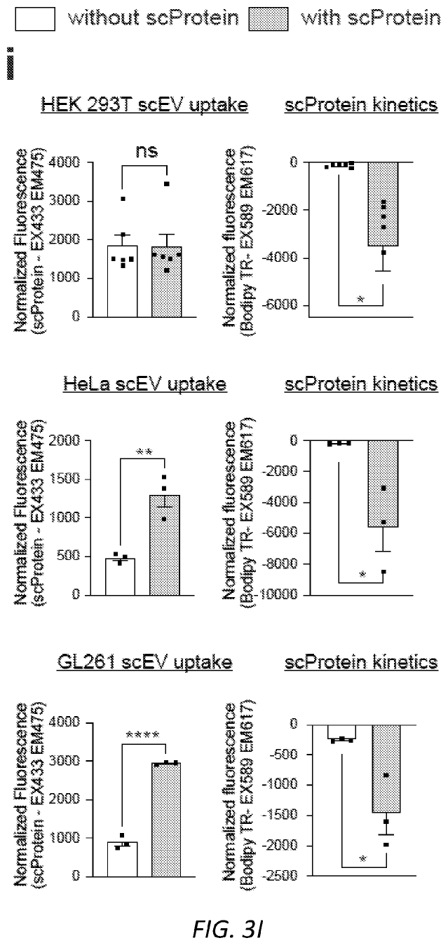

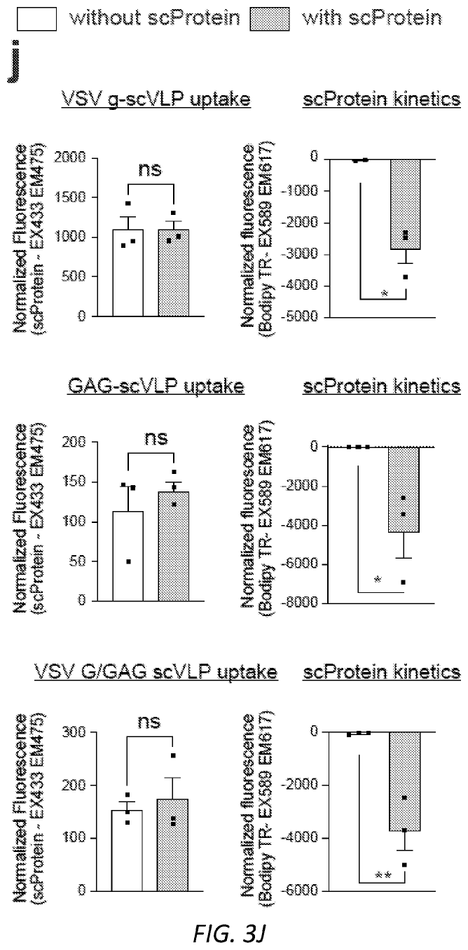

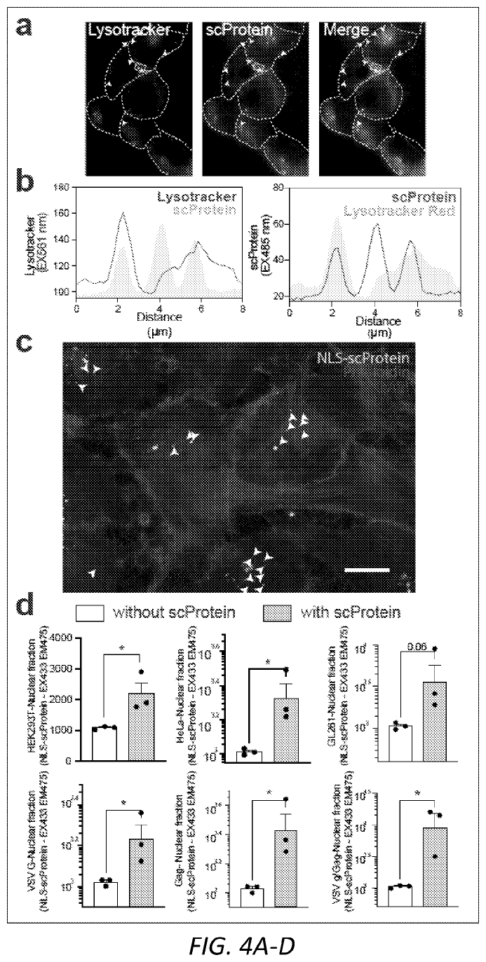

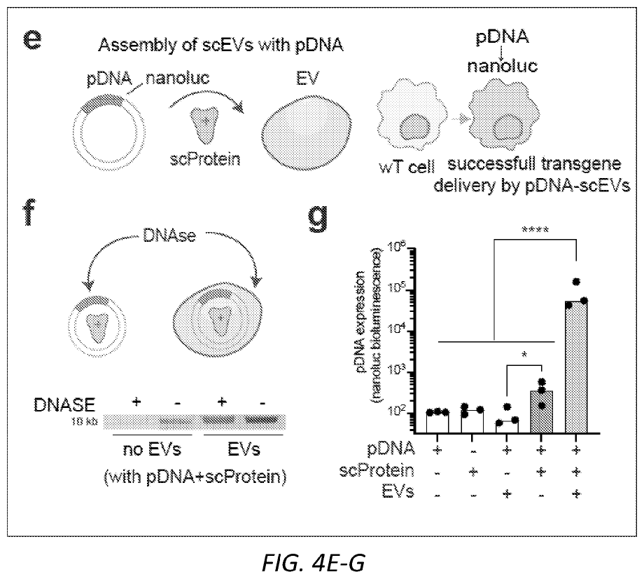

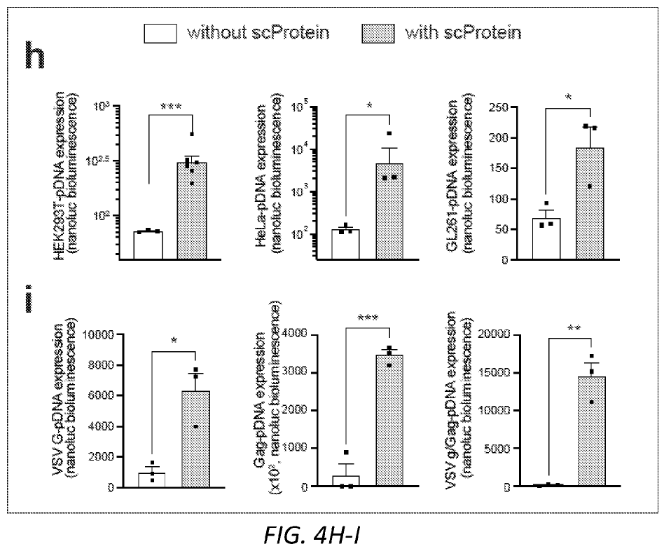

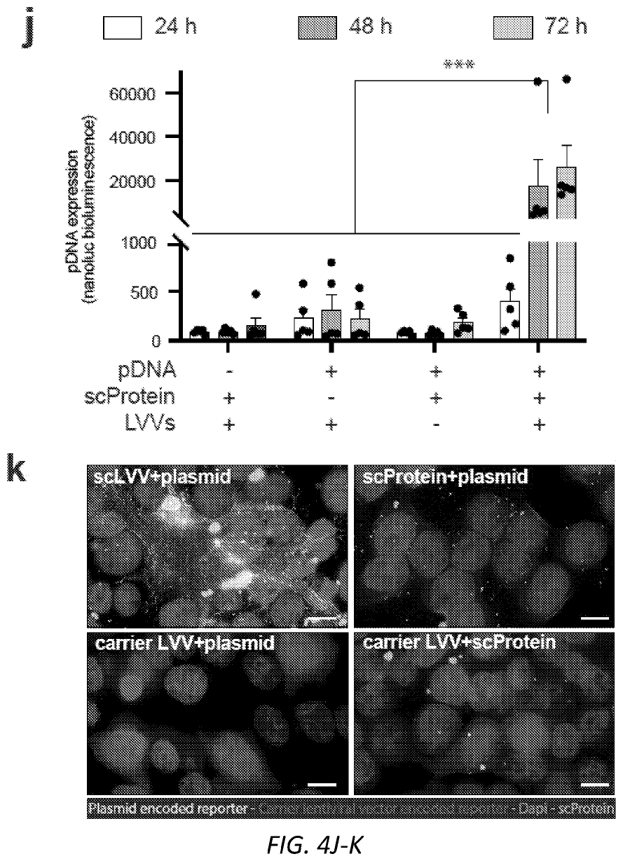

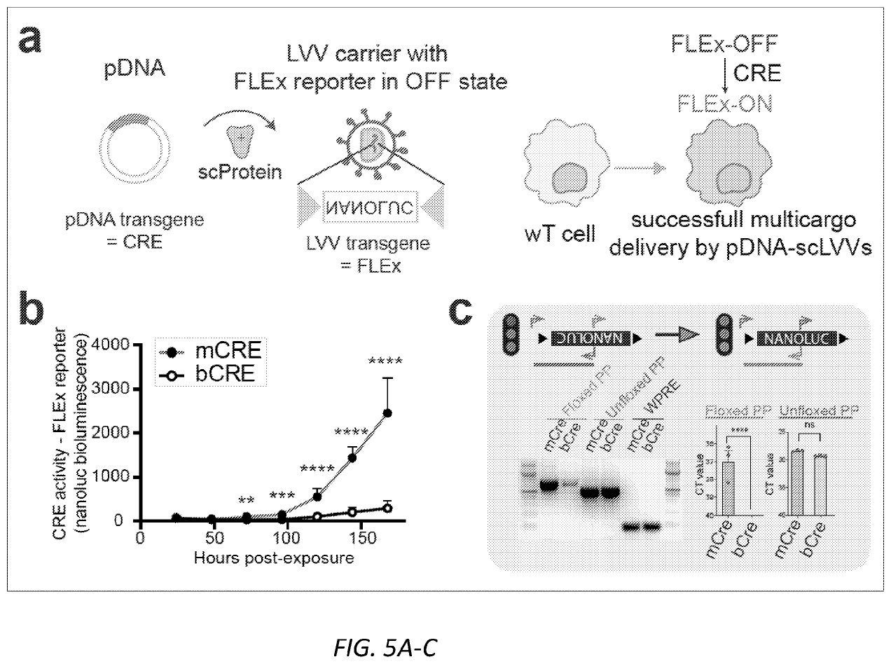

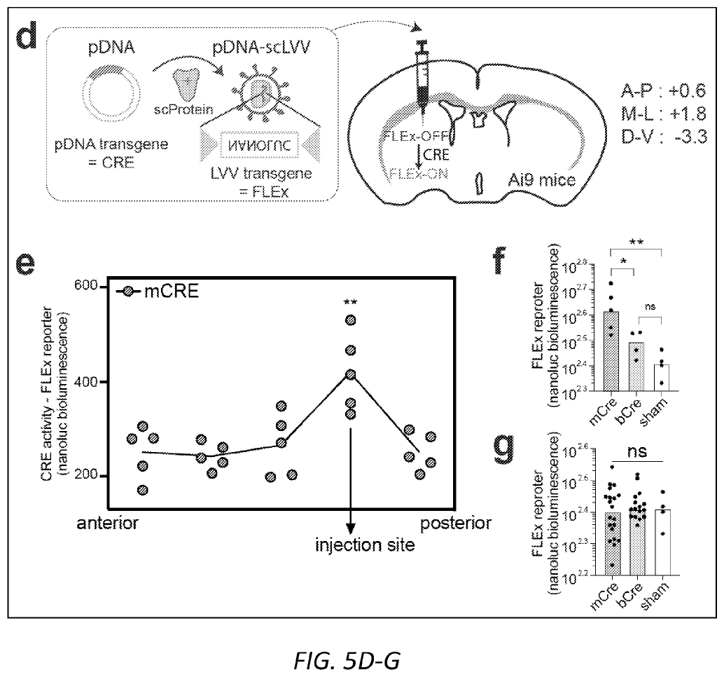

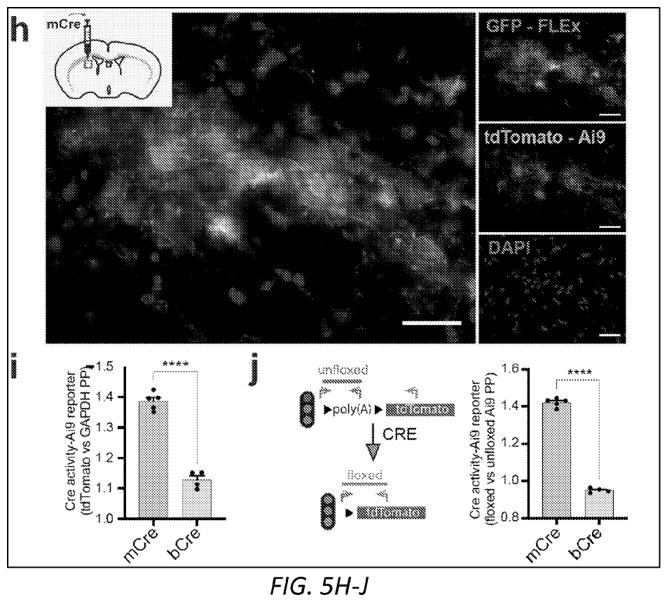

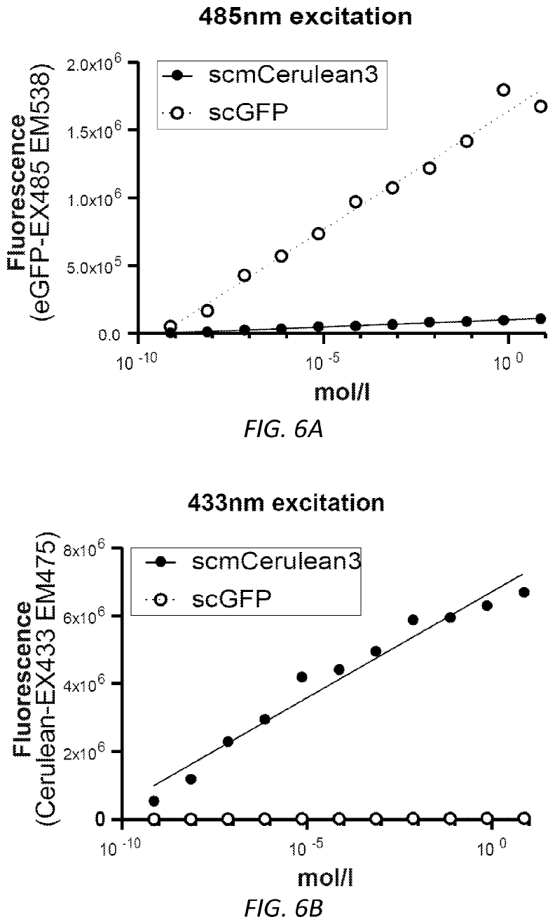

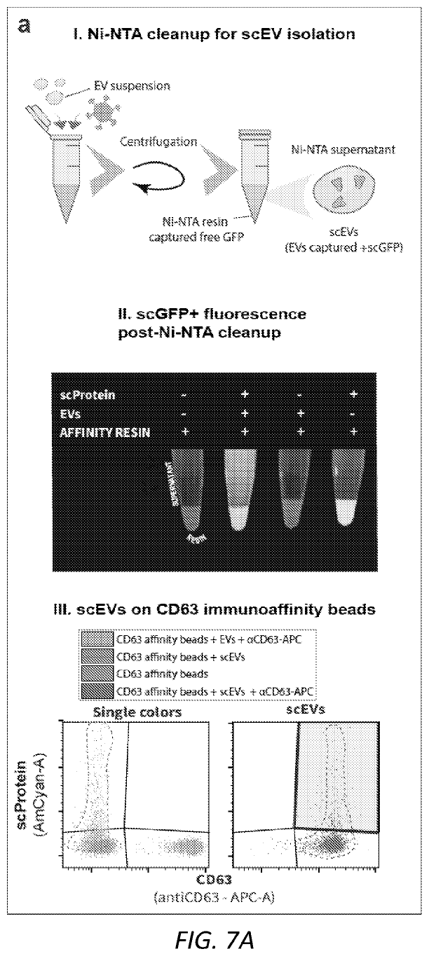

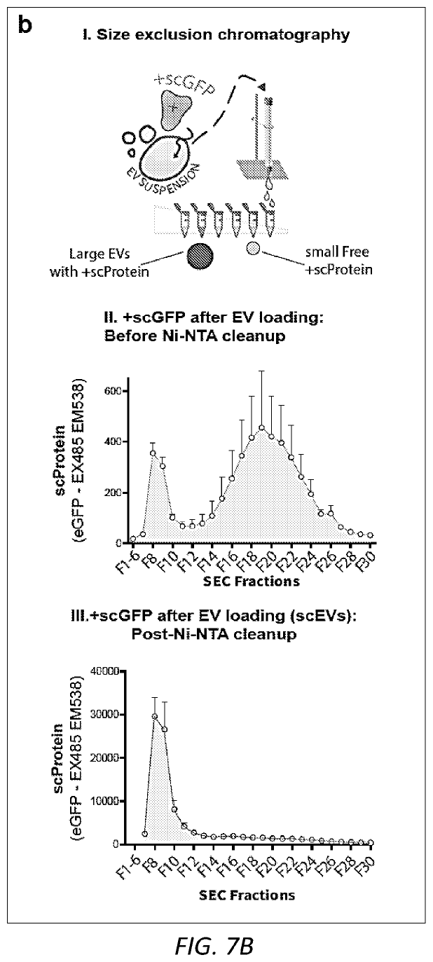

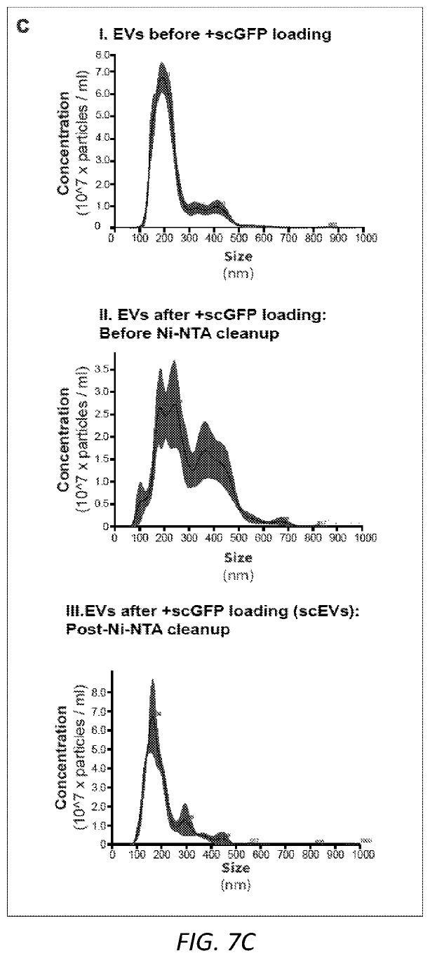

Green fluorescent protein (GFP) has a beta barrel scaffold with a center chromophore¹⁶. Through site-specific mutagenesis of the center structure, the fluorescent spectrum of this fluorescent protein can be switched from green emission (EX485-EM538) to cyan emission (EX433-EM475)¹⁷ (FIG. 6), while site-directed mutagenesis of the exposed surface of GFP altered the biophysical properties of the molecule (FIG. 1-A). The net charge of GFP can be decreased or increased by modulation of the lysine/arginine ratio¹⁸, generating a negatively or a positively supercharged protein (scProtein) (FIG. 1-B). To evaluate the affinity of proteins with different net charges to EVs, three recombinant His-tagged eGFP homologues (−scGFP, eGFP, and +scGFP—amino acid sequences below) were generated and evaluated for their binding with His-tag affinity Ni-NTA resin (FIG. 1-C). The Ni-NTA resin was observed to capture 99.1±0.13%, 98.8±2.1%, and 99.3±0.19% of detectable fluorescence intensity from suspensions of recombinant -scGFP, eGFP, and +scGFP, respectively (FIG. 1-D). However, when −scGFP, eGFP, and +scGFP recombinant proteins were incubated with purified EVs from HEK293T cells (FIGS. 1-E), 99±0.3%, 96.1±7.1%, and 55.9±6.8% of the GFP fluorescence remained in suspension, respectively (FIG. 1-F). We hypothesized that EV association prevented the +scGFP His-tag from binding to Ni-NTA, generating an EV assembly, here called supercharged EVs (scEVs). In essence, the Ni-NTA capture of unloaded free recombinant +scGFP (FIG. 7-A) allowed us to generate a fluorescent +scEV suspension (FIG. 7-A), whereby the +scGFP co-immunoprecipitated with CD63 immuno-affinity beads indicative of +scProtein association with EVs (FIG. 7-A). We verified this hypothesis by resolving the +scGFP fluorescent suspension after Ni-NTA cleanup by Size Exclusion Chromatography (SEC) (FIG. 7-B). EVs are relatively large particles (30-200 nm)¹⁹ that peak in the low-numbered SEC fractions (F7-11), while signal retrieved in the later SEC fractions (F13-F28) represent free +scProteins (2-3 nm). This can be demonstrated when resolving a +scGFP solution (without EVs) with SEC. In this case, the fluorescent signal was only detected in the later fractions (FIG. 1-G). When +scGFP was mixed with non-fluorescent SEC-purified HEK293T EVs (FIG. 1-H) for 45 min prior to SEC, the fluorescence could be detected as two peaks (FIG. 7-B). The F13-F28 SEC fraction peaking in the latter profile was lacking in a SEC profile post-Ni-NTA cleanup (FIG. 1-I & FIG. 7-B). This confirmed our interpretation that Ni-NTA cleanup ensures +scProtein-associated EV purification. Moreover, we explored whether our method also reduced potential aggregate formation in our suspension that might be formed due to mixing of +scGFP with EVs (FIG. 7-C). With Nanosight particle analysis of EVs before (FIG. 7-C) and after +scProtein loading (FIG. 7-C), a shift in profile was seen from a homogenous one peak profile to a two-peak profile. The Ni-NTA cleanup to generate scEVs excluded aggregates resulting in a profile expected for a single vesicle suspension (FIG. 7-C).

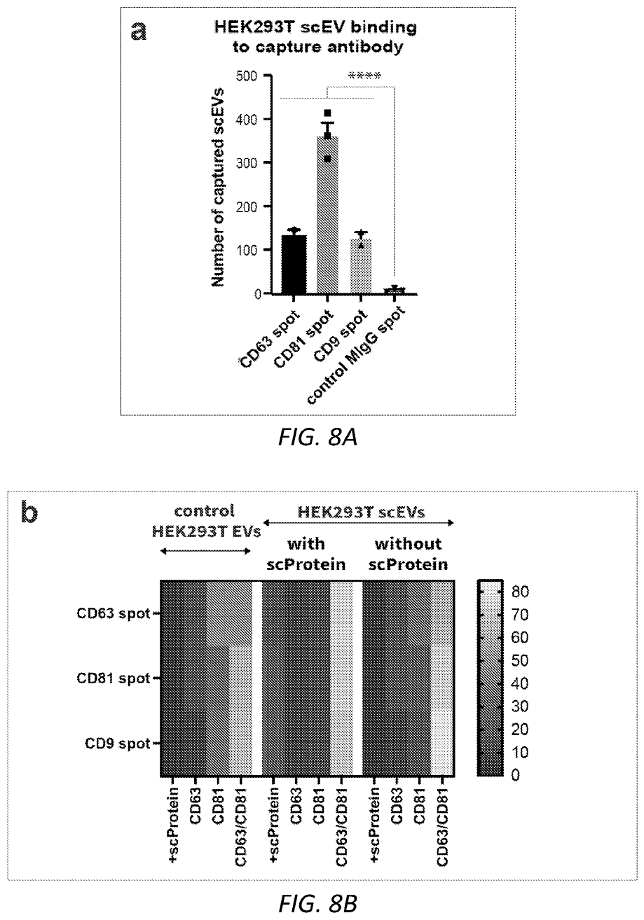

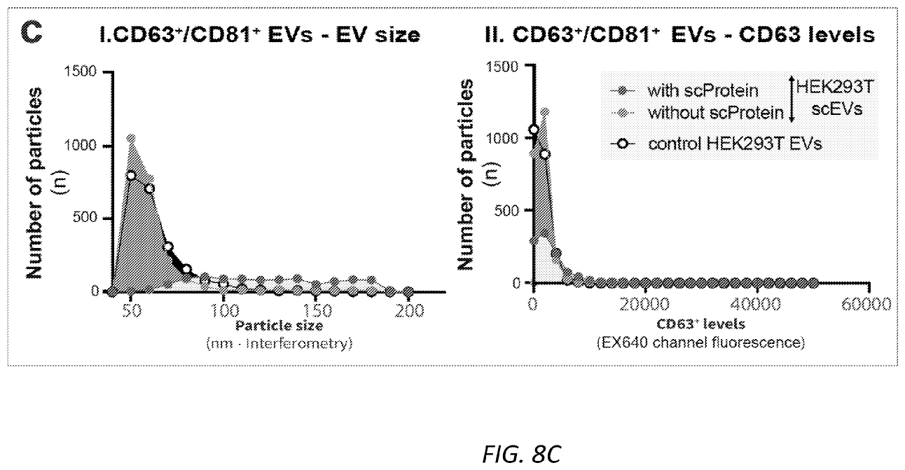

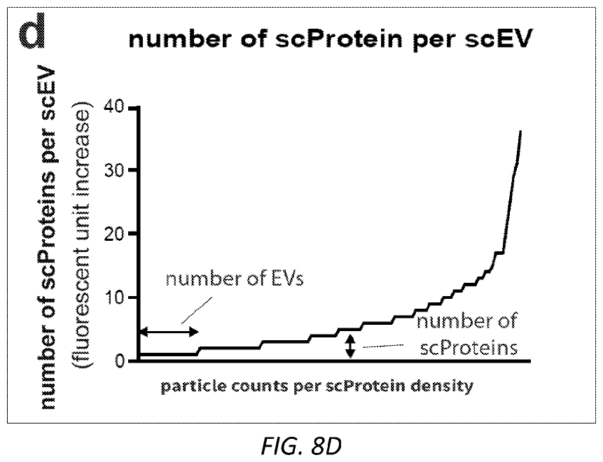

The unique affinity of isolated EVs for +scProteins was further demonstrated by the dependency of +scGFP capture on the number of EVs in solution. When a higher number of SEC-purified HEK293T EVs were provided to the same amount of +scGFP (15 pmol), an increase in +scGFP fluorescence was observed (FIG. 1-J). The correlation between the number of scEVs and the increase in fluorescence due to +scGFP association is Y=20.36+0.8214X+0.004077 X² (R2=0.99; p<0.005), where Y represents the scProtein emitted fluorescence and X the number of 10⁵ EVs. A population view of our resulting scEV suspension was investigated with ExoView® analysis²⁰ (FIG. 1-K). scEVs were captured on anti-CD63, anti-CD81, or anti-CD9 printed in distinguishable spots on an ExoView®Chip and compared to an isotype IgG control spot. On a capturing antibody spot, a single EV is bound allowing a multiparametic comparison through colocalizing fluorescent signals of +scProtein in the blue channel and two detection antibodies, anti-CD63 in the red channel and anti-CD81 in the green channel (FIG. 1-L). Specific scEV binding to our ExoView®Chip was confirmed as 130.5±21.9, 361.3±52.5, 126±21.2 fluorescent +scProtein events detected on the anti-CD63, anti-CD81, anti-CD9 capture spots, respectively compared to 7±4.6 events on the IgG control spot (FIG. 8-A). We also compared a non-loaded HEK293T EV sample with a HEK293T scEV sample (FIG. 8-B). In our scEVs sample, we observed that the sample contained both EVs that had absorbed +scProtein and EVs that had not, with 8.2%±2.0% of the EVs being +scProtein positive. The non-exposed +scProtein exposed EV sample had a similar heatmap profile compared to EVs without +scProtein in our scEV sample. However, EVs with +scProtein in the scEV sample were enriched in an EV subpopulation containing both CD81 and CD63. With interferometric sizing measurements (IM) at each capturing antibody we determined that the level of +scProtein fluorescence is dependent of the size of the loaded EV. The linear correlation is log₂(Y)=0.03*X+7.7 (R2=0.81; p<0.0001), where Y represents the +scProtein emitted fluorescence and X the size of EVs (FIG. 1-M). The slope of this correlation (slope=0.03) was higher than when comparing CD81 (slope=0.019) and CD63 (slope=0.019) detection antibody emitted fluorescence with IM (FIG. 1-N). Also, the R2 value stipulating the strength of the relationship between the fluorescence and the IM was lower for CD81 (R2=0.34) and CD63 (R2=0.32). Altogether, our data indicates that CD63 and CD81 were incorporated in a similar fashion into EVs by the donor cell and that exogenous +scProtein loading is more size dependent compared to endogenous loading of tetraspanins. This observation was confirmed when comparing the EV size indicated by IM (FIG. 8-C) and anti-CD63 probe fluorescence (FIG. 8-C) of CD63⁺CD81⁺events between non-loaded EV samples and loaded scEV samples. Indeed, in the CD63⁺CD81⁺subpopulation the size of single EVs was larger with +scProtein (70-190 nm) compared to EVs without +scProtein and unloaded control EVs (50 nm-90 nm), while the CD63 fluorescence was the same. The latter observation supports inclusion of single EVs in our analysis. We calculated that 7-10% of the EVs in our +scProtein-exposed EV sample were loaded and 81.8% of the EVs with +scProtein had between 1 to 10 scProteins; 13.6% had between 10 to 20 scProteins; 2.7% had between 20 to 30 scProteins; and 1.8% had more than 30 scProteins (FIG. 8-D).

### Example 1.2 Extracellular Vesicles Properties Aid in Generating Supercharged Extracellular Vesicles

Our results support the ability of positively charged residues of +scProtein to associate with BVs, such as EVs. Here, we investigated whether EV properties also aid in +scProtein association. Bio-Beads SM2 are nonpolar polystyrene adsorbents which are not expected to bind to hydrophilic +scGFP (FIG. 2-A) but should be able to pellet phospholipids²¹ present on EVs. When these Bio-Beads were mixed with free +scGFP in solution 71.5±15.2% of the fluorescence stayed in suspension (FIG. 2-B). However, when scEVs were mixed with Bio-Beads SM2 (FIG. 2-C), the fluorescence was reduced to 11.9±13.5% in suspension confirming the capture and pelleting of +scGFP by cell-derived biomembranes present in EVs (FIG. 2-D).

We explored whether the lipids in EV membranes are the main determinant underlying association with +scProtein. Therefore, we compared +scProtein association with liposomes consisting of dipalmitoylphosphatidylcholine-polyethylene glycol 2000—distearoylphosphatidylethanolamine-cholesterol with a known surface charge of −11±1 mV to that of EVs (FIG. 2-E). After Ni-NTA pull-down, the +scProtein fluorescence in suspension with EVs was 6.29-fold higher compared to the liposome condition and 6.06-fold higher than the no vesicle control (scProtein alone) (FIG. 2-F). We hypothesized that negative charges on the surface of BVs might be sugar moieties, which are lacking in liposomes. Therefore, EVs were treated with different enzymes, such as PGNase F or different heparinases (I, II and III), to remove N-glycosylated moieties and thereby reduce the negative charge of their biological membranes²² (FIG. 2-G). These enzyme treatments significantly decreased the +scProtein fluorescence in the supernatant after Ni-NTA pull-down, indicative of the level of +scProtein capture by EVs (FIG. 2-H). Compared to non-treated samples a 74.1-, 69.13-, 84.70-, 92.34-, and 112.5-fold decrease in the supernatant was observed for a pretreatment with heparinases (I, II and III), PGNase F and a mixture of Heparinase III with PGNase F, respectively (FIG. 2-I). As a consequence of the decreased absorbance of +scProtein by EVs, an increase in +scProtein fluorescence in the Ni-NTA pellet was observed for the deglycosylated EV conditions. Compared to non-treated samples a 3.16-, 3.68-, and 3.58-fold increase was observed for a pretreatment with heparinases (I, II and III), PGNase F and a mixture of Heparinase III with PGNase F, respectively. Deglycosylation also enabled us to evaluate the stability of our established scEV complex. Here, we remove sugar moieties after +scProtein association with EVs, instead of our previously performed EV deglycosylation before +scProtein loading. If the sugar moieties on the extraluminal side merely mask the +scProtein interaction with the Ni-NTA, we expected that +scProtein on scEVs would detach after deglycosylation (FIG. 2-J). However, if the association of +scProtein with scEVs was stable, a deglycosylation treatment would have no effect (FIG. 2-K). No significant additional pull-down of Ni-NTA was observed after deglycosylation of scEVs with heparinases (I, II and III) or PGNase F compared to untreated scEVs (FIG. 2-L). These results substantiate that removing oligosaccharides after loading +scProtein does not influence the stability of the scEV assembly.

### Example 1.3 Uptake of Supercharged Extracellular Vesicles in Cultured Cells

Uptake of EVs by recipient cells can be monitored with membrane dye labelled EVs²³. To explore the uptake of scEVs by recipient cells, we fluorescently labelled scEVs with a Bodipy TR ceramide membrane dye²⁴ (FIG. 3-A). To generate our Bodipy TR-scEVs, EV samples were incubated with dye prior to supercharging EVs (FIG. 9-A). Our method included resolving a Bodipy TR labelled EV suspension over a SEC column removing free dye visible in the higher non-EV SEC fractions (FIG. 9-B) and a 100 kDa/˜10 nm cut-off filter treatment to clean up any potential free +scProtein remaining in our suspension. Free +scProtein was demonstrated to pass through a 100 kDa cut-off filter but could be retained by a 30 kDa/˜3 nm cut-off filter (FIG. 9-B). Of note, non-supercharged Bodipy TR-EV controls were generated from the same EV source sample as our Bodipy TR-scEVs avoiding batch-to-batch variations between EV production (FIG. 9-A). In our final suspension, the BODIPY TR-label co-immunoprecipitated with CD63 affinity beads, known to bind CD63⁺EVs, as did the +scProtein fluorescent signal (FIG. 9-C).

When exposing HEK293T Bodipy TR-scEVs to HEK293T cells cultured in EV-free media, an increase in Bodipy TR cell-associated fluorescence was observed over a 200 min (1 measurement/10 min) monitoring experiment (FIG. 3-B). The uptake of the non-supercharged Bodipy TR-EV controls was not significantly different than their Bodipy TR-scEV counterparts. Half of the Bodipy TR signal was reached after 170.8-227.5 min (R2=0.90) and 222.9-433.3 (R2=0.75) min for EVs and scEVs, respectively. Concomitantly with Bodipy TR fluorescence, we monitored +scProtein fluorescence as a measure of the half-life of the recombinant +scProtein (FIG. 3-C). A decrease in +scProtein fluorescence was significantly greater (p<0.01) when the recombinant protein was exposed to HEK293T cells without EV-association than when it was delivered in the scEVs format. The half-life of +scProtein without association with EVs was 25.4-42.00 min (R2=0.53), in contrast to 65.9 to 105.8 min (R2=0.73) with scEVs.

To examine the potential mechanisms of HEK293T scEV uptake, we pretreated HEK293T cells with inhibitors targeting the endolysosomal and autophagic pathways before adding Bodipy TR-scEVs. v-ATPase inhibitors concanamycin A (ConA, 200 nM) and Bafilomycin A1 (Baf, 200 nM) were used to investigate whether scEVs act through a potential pH-dependent uptake mechanism. Inhibitor pretreated HEK293T cells (1 h) were investigated 24 h post-exposure to Bodipy TR-scEVs with flow cytometry. No significant decrease in Bodipy TR was observed compared to sham-pretreated cells (FIG. 3-D). However, pretreatment with a lysosomotropic agent, such as Chloroquine (CHL, 10 μM)²⁵ for 1 h did significantly increase Bodipy TR signal fluorescence (p<0.0001), indicating scEV accumulation in the recipient cells. Bortezomib (Bort, 5 μM), a 26S proteasome inhibitor, which also induces autophagy²⁶ and ER stress^{27,} increased scEV uptake compared to sham (p<0.001). These inhibitors have been interchangeably used to track both particle uptake as lysosomal protease activity in cells²⁸. Therefore, +scProtein fluorescence levels were measured and demonstrated to increase only compared to sham when pretreated with CHL or Bort (p<0.0001, FIG. 3-E). To investigate whether +scProtein fluorescence in cells is solely increased with Bort or CHL pretreatment by an elevated scEV uptake, we combined both treatments (FIG. 9-D). Post-Bodipy TR-scEV uptake, Bodipy TR signal was not affected by the combination pretreatment (FIG. 9-D), while +scProtein fluorescence in cells increased when treated with both Bort and CHL compared to CHL alone (FIG. 9-D, p<0.05). These observations suggest that +scProtein kinetics in a scEV recipient cell are not only dependent of scEV uptake and can be enhanced after scEV uptake by blocking protease activity in recipient cells with Bort. Of note, the cytoplasmic proteasome inhibitor Bort also increased +scProtein fluorescence in combination with Baf and ConA (P<0.01). HEK293T Bodipy TR-scEVs were separated based on particle size through serial filtration with 450 nm, 220 nm, 100 nm, 30 nm, and 10 nm cut-off filters to examine the effect of size (FIG. 3-F). In contrast to free scProtein, which was only retrieved in a 3 nm cut-off filter (FIG. 9-B), an scEV solution could be concentrated by 100 nm, 30 nm, and 10 nm cut-off filters thereby obtaining 100 nm to 220 nm, 30 nm to 100 nm, and 10 nm to 30 nm sized HEK293T Bodipy TR-scEVs, respectively. Based on Bodipy TR fluorescence, no significant difference in cell uptake was observed between different sized-Bodipy TR scEVs (FIG. 3-G). In contrast, +scProtein half-life differed based on Bodipy TR-scEV particle size (FIG. 3-H). In essence, larger (100 nm to 220 nm) scEVs had an intracellular half-life of 25.3 to 61.0 min (p<0.01, R2=0.75) compared to the smaller (10 nm to 30 nm, and 30 nm to 100 nm) scEVs with a half-life of 7.1 to 43.6 min (p<0.0001, R2=0.73). This data suggests that smaller scEVs which carry less +scProtein (FIG. 1-M) are potentially more susceptible to cell endosomal degradation compared to larger scEVs with larger payloads.

In contrast to the use of homologous scEVs, Bodipy TR-scEVs from other cell sources were tested for their internalization potential on common recipient HEK293T cells. HeLa EVs and GL261 EVs had a similar Nanosight Analysis profile before being labelled with Bodipy-TR, supercharged with +scProtein, and exposed to HEK293T cells (FIG. 9-E). Based on Bodipy-TR fluorescence, HeLa EVs (p>0.05) and GL261 EVs (p<0.05) did not well accumulate over a 200 min incubation at 37° C. with HEK293T cells compared to control (grey graph compared to black graph in FIG. 9-F). However, when GL261 EVs (p<0.0001) and HeLa EVs (p<0.0001) were supercharged, their uptake by HEK293T cells was increased significantly compared to unsupercharged GL261 EVs and HeLa EVs (FIG. 9-F). When comparing to homologous uptake of scEVs with heterologous scEV uptake by HEK293T cells, Bodipy TR fluorescence increases in the latter with supercharging (FIG. 3-I—left bar graphs, FIG. 9-F and H) but does not influence the +scProtein fluorescence in the cell (FIG. 3-I—right bar graphs & FIG. 9-H).

To verify whether in addition to the origin of EVs, viral factors could influence the uptake of supercharged BVs, virus-like particles (VLPs) were generated from HEK293T cells by overexpression of VSV G, GAG, or both VSV G and GAG (FIG. 9-E). Similar to what we earlier observed with HEK293T EVs, no significant difference in uptake between Bodipy TR labelled supercharged and non-supercharged HEK293T VLPs was observed when incubated with HEK293T cells (left graphs in FIG. 3-J and FIG. 9-G). +scProtein fluorescent levels delivered with scVLPs did not differ between conditions in recipient HEK293T cells (right graphs in FIG. 3-J and FIG. 9-G).

Altogether, our data indicates that scEV uptake and scProtein kinetics in recipient cells can be influenced by particle size and EV source, but not by endogenous EV loading of viral components.

### Example 1.4 Leakage of scProtein from Lysosomal Compartments of Supercharged Extracellular Vesicles and Supercharged Viral Like Particles in Recipient Cells

Our previous observations with inhibitors of endolysosomal function suggested this route of EV internalization by cells. Here, we marked the low pH compartments in a scEV-recipient HEKT293T cell with LysoTracker Red29 and investigated its position compared to +scProtein (FIG. 4-A). Seventy-two h after scEV exposure, we determined that scProtein was present in both Lysotracker positive and Lysotracker negative cell compartments (FIG. 4-B). To confirm +scProtein leakage from endosomes post-scEV exposure, we loaded +scProteins equipped with a nuclear localization signal (NLS) into EVs. The resulting NLS-scEVs were incubated for 3 days with HEK293T cells and screened for scProtein fluorescence in the nucleus (FIG. 4-C). Isolated nuclei from recipient HEK293T cells after 4 days of NLS-scEVs or NLS-scVLPs exposure were then compared to nuclei of HEK293T cells exposed to EVs and VLPs from the same EV batch, but which had not been supercharged (FIG. 4-D). A significant difference in +scProtein fluorescence of the isolated nuclei was observed for HEK293T scEVs (p<0.05), HeLa scEVs (p<0.05), VSV G-scVLPs (p<0.05), GAG-scVLPs (p<0.05), or VSV G/GAG-scVLPs (p<0.05), while for GL261 scEVs we only noted a trend (p=0.06) compared to non-supercharged EV or VLP controls. In line with +scProtein accumulation in cells between different EV sources (FIG. 9-H), no differences were observed for nuclear translocation between HEK, HeLa and GL261 scEVs. In contrast, loading multiple viral components such as VSV G and GAG increased nuclear translocation compared to GAG alone (p<0.05). These results indicate again that scEV uptake by cells does not predict +scProtein kinetics.

### Example 1.5 Supercharged Extracellular Vesicles, Supercharged Viral Like Particles, and Supercharged Lentivirus Vectors Mediate Plasmid DNA Expression in Recipient Cells

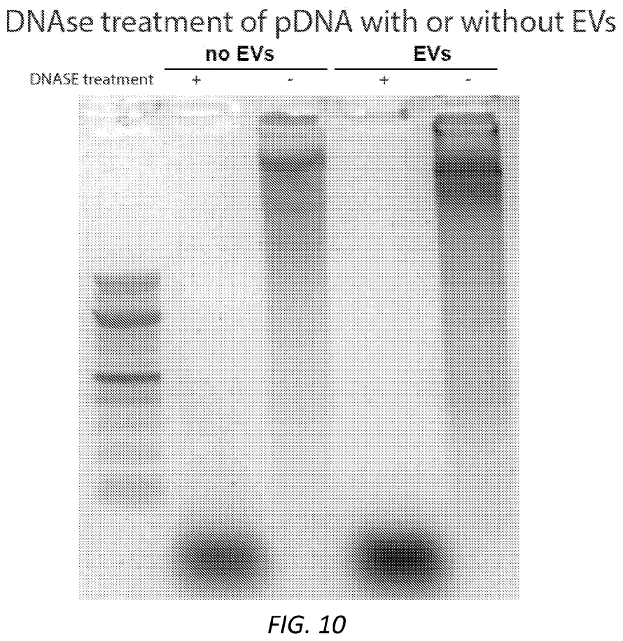

It has been reported that plasmid DNA (pDNA) can be piggybacked into cells with scProteins¹⁵. We tested whether EVs associated with both +scProtein and pDNA would generate a pDNA encoded signal in the pDNA-scEVs recipient cells (FIG. 4-E). The plasmid used for these experiments encoded a nanoluciferase reporter (see Table 1) that enables bioluminescent recordings upon expression by cells. We tested the stability of the pDNA-scEV assembly using DNase I treatment which was able to degrade pDNA in the presence of EVs without +scProtein (FIG. 10). We observed that when pDNA is associated with +scProteins, the pDNA is still susceptible to DNase I treatment, but not when that combination is assembled with EVs as a pDNA-scEV complex (FIG. 4-F). The HEK293T pDNA-scEVs assembly, containing all three components (EVs, scProtein, and pDNA), was incubated with HEK293T cells for 4 days (FIG. 4-G). pDNA expression in the recipient cells was established by a detectable bioluminescence signal. This signal was significantly higher compared to other conditions whereby cells were exposed to only two components (+scProtein and pDNA or EVs and pDNA, p<0.0001) or to single components (pDNA or EVs, p<0.0001). As published in earlier reports³⁰, the condition without EVs (pDNA and scProtein) was also able to significantly increase the bioluminescent signal compared to condition without scProtein (pDNA and EVs), but to a 4.5-fold lower extend than our pDNA-scEVs assembly (p<0.0001). Moreover, we evaluated whether this pDNA-mediated delivery of HEK293T pDNA-scEVs was solely restricted to HEK293T EVs (FIG. 4-H). Therefore, GL261, HeLa or HEK293T derived EVs were used to generate pDNA-scEVs and control suspensions (pDNA and EVs without +scProtein). Based on bioluminescent readings, GL261 pDNA-scEVs (p>0.05) and HeLa pDNA-scEVs (p>0.05) were also able to induce pDNA expression in HEK293T cells after 4 days of incubation compared to the control suspensions without +scProtein but with pDNA and GL261 EVs or HeLa EVs, respectively. Similar conclusions to the NLS-scEV experiments, there was no difference in nanoluc expression observed between HEK293T, HeLa, or GL261 pDNA-scEVs observed. The same observation was made with HEK293T pDNA-scVLPs and HEK293T recipient cells (FIG. 4-I). VSV G-scVLPs (p<0.05), GAG-scVLPs (p<0.001), or VSV G/GAG-scVLPs (p<0.01) increased bioluminescence in HEK293T cells compared to control suspensions without +scProtein, but with pDNA and VSV G-VLPs, GAG-VLPs, or VSV G/GAG-VLPs, respectively. No significant differences were observed between the different scVLPs conditions in terms of nanoluc bioluminescence. We also investigated whether HEK293T LVVs (Lenti Virus Vectors) carrying a mCherry transgene could be complexed to +scProteins and pDNA generating pDNA-scLVVs. Different conditions consisting of combinations of LVV, pDNA, and +scProteins were exposed to HEK293T cells for 3 days (FIG. 4-J). Only the pDNA-scLVV assembly with all components (scLVV=+scProtein, LVV, and pDNA) being present was able to express detectable levels of bioluminescence. The pDNA-scLVV induced signal was 119-169-fold higher as compared to controls. Data could be confirmed with immunohistochemistry detecting Flag-tag present in our pDNA encoded protein in less than 0.01% of the pDNA-scLVV recipient cells, and absent in cells after control treatments (FIG. 4-K). Overall, our data confirm that +scProteins assembled with EVs, VLPs, or LVV and pDNA mediate pDNA expression in recipient cells.

### Example 1.6 Supercharged Lentiviral Vectors as a Model for a Future Multi-Cargo Delivery Modality in the Brain

Our previous findings indicate that pDNA-scL VVs deliver both LVV- and pDNA-encoded transgenes using mCherry and a Nanoluc reporter, respectively. Here, we tested whether both transgenes (pDNA-transgene and LVV-transgene) can successfully be delivered to the same recipient cell and collaboratively generate a bioluminescent signal (FIG. 5-A). We developed a model with a LVV that encodes a nanoluciferase-reporter transgene here called FLEx. FLEx in our LVV is in the OFF-state and can only be activated to the ON-state when pDNA encoding for Cre recombinase is co-delivered. We tested whether FLEx activation is possible with pDNA-scLVVs, when LLVs are carrying the FLEx-OFF transgene. We compared in these experiments pDNA-scLVV loaded with a Cre plasmid preceded by either a mammalian promoter (mCre—test) or a bacterial promoter (bCre—control). As expected, only the pDNA-scLVV with the mCre could induce FLEx-reporter activity in our recipient HEK-293T cells (p<0.0001, FIG. 5-B). We confirmed activation of the FLEx reporter in the mCre condition, absent in the bCre condition, with qPCR utilizing primer pairs discriminating between the ON and OFF state of FLEx at the gDNA-level in the recipient cells (FIG. 5-C). Of note, under both pDNA-scLVV conditions (mCre and bCre) similar amount of LVV transgene integration (p>0.05) could be detected in the genome of the recipient cells as assessed by qPCR for WPRE.

We revisited our FLEx model to test whether pDNA-scL VV can co-deliver two payloads (pDNA transgene and LVV transgene) to brain cells (FIG. 5-D). mCre- or bCre-encoding pDNA-scLVVs suspensions were injected into the striatum of adult Ai9 reporter mice31, in which Cre can turn on tdTomato in mouse cells. Twenty-five days after injecting pDNA-scLVVs, Ai9 mice were sacrificed and checked for Cre and FLEx deployment in the brain. Sections (80 um) were taken from different areas in the Ai9 brains to verify FLEx-reporter delivery and FLEx-activation as a result of Cre activity. We confirmed FLEx activity in the section at the injection site as the nanoluciferase signal was significantly higher (FIG. 5-E, p>0.01) compared to sections in anterior or posterior brain regions. Furthermore, Ai9 animals receiving bCre or mCre pDNA-scLVVs were compared at the injection site (FIG. 5-F) and more distal brain regions (FIG. 5-G). FLEx-activity was significantly higher for mCre as compared to bCre or sham injected animals (p>0.05), while no difference was observed in nanoluciferase signal in other sections among the conditions (p>0.05). To confirm that Cre delivered by pDNA-scLVVs was not only able to flip our FLEx-switch, we also screened for tdTomato signal that results from activation of the mouse-encoded Ai9 reporter. pDNA-scLVV delivery to brain cells was identified at the injection site through GFP fluorescence encoded by the FLEx-transgene (FIG. 5-H). In these cells, Ai9 tdTomato fluorescence was visualized with immunohistochemistry. We quantified the tdTomato transcript levels at the injection site in pDNA-scLVV injected animals and thereby showed that the Ai9 reporter was significantly activated by mCre, and not by bCre (p>0.0001, FIG. 5-I). We confirmed that the tdTomato signal resulted from removal of a floxed region in Ai9 mice by Cre activity (FIG. 5-J). Indeed, qPCR with discriminating primers between floxed and unfloxed regions confirmed floxing by Cre in Ai9 brain sections with mCre pDNA-scLVV compared to bCre pDNA-scLVV (p>0.0001). As exemplified with pDNA-scLVVs, our data suggest that the delivery capacity of vesicles can be augmented through supercharging to deliver multiple types of biomolecules with a single carrier platform that work in a combinatorial way on recipient cells.

### Example 2. Extracellular Vesicles a Tattletale for Rare Gene Editing Events

Since its discovery, gene editing has provided the ability to meticulously change genes with a profound effect on both therapeutics and molecular research. Even with new tools constantly being developed to increase efficiency and precision of the technique, the repair mechanisms post-gene editing are still error prone making it critical to detect and/or select a desired gene corrected cell clone. Since the contents of extracellular vesicles (EVs) reflect the cells that produced them, if a gene editing event occurs, the EV cargo should contain the gene corrected products, such as a protein or RNA species. The catch lays in the fact that EVs are by their nature very heterogeneous and only a small fraction of the population may harbor the gene edited products.

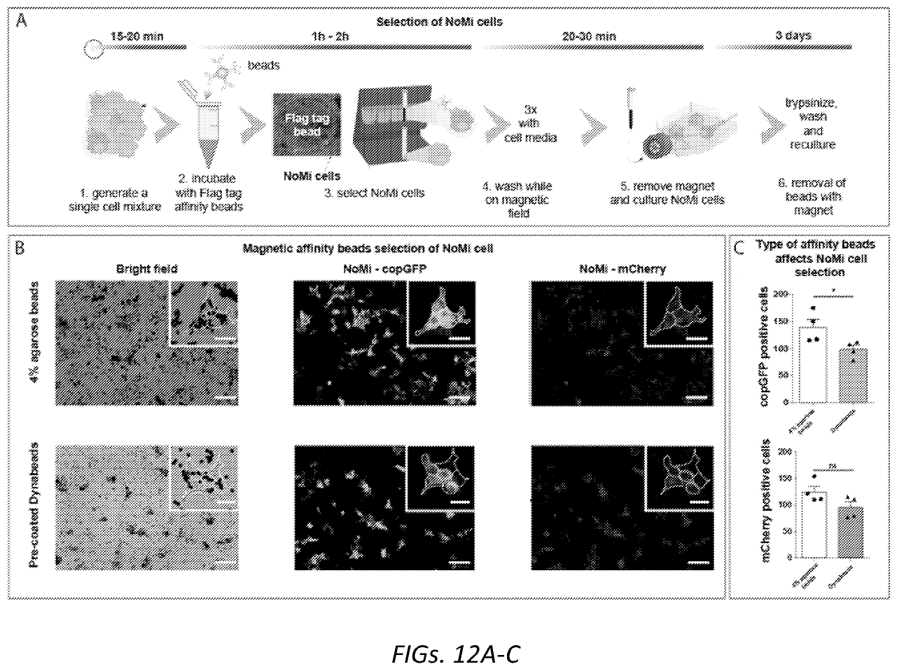

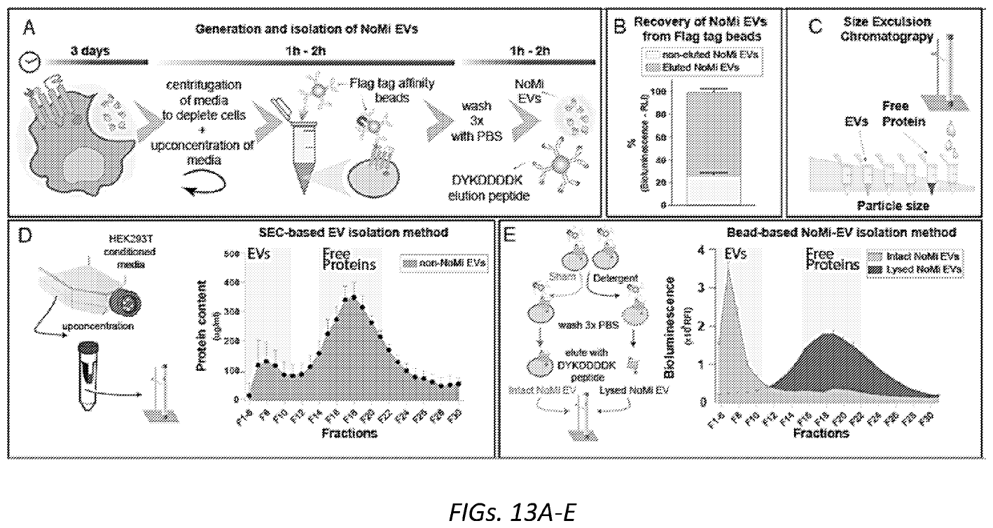

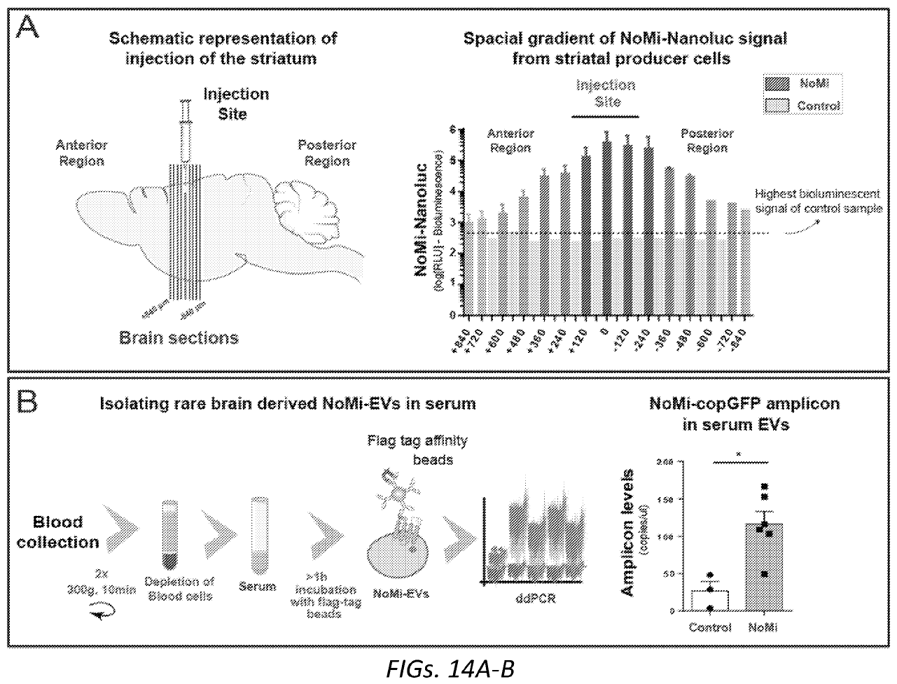

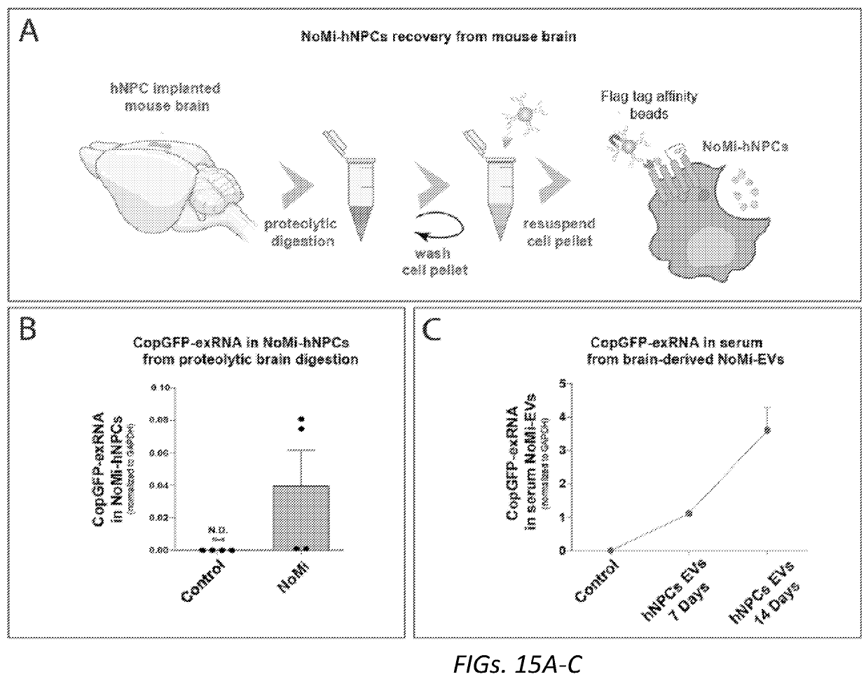

We designed a CD63 construct with a genomic DNA target sequence for detection of a desired gene editing event. See FIG. 11. The gene editing target harbors a premature stop codon. Only when the desired gene editing event occurs to correct stop codon truncations by genomic missense or frameshift mutations, is a bioluminescent signal detected, as it then allows the CD63 tethered luciferase reporter to be translated. See, FIG. 12A.

As shown in FIG. 12B, this reporter detected gene corrections 2 days post-introduction of Cas9 and a sgRNA targeting the stop codon. Next-generation sequencing confirmed that the signal resulted from 1.12% gDNA changes. Our observations highlight the sensitivity of our system to detect even highly inefficient non-homologous end joining repair after a double-strand break within the target DNA. The CD63 construct also contained a membrane surface immune affinity tag to facilitate isolation of cells that encode the full-length reporter, excluding the non-gene edited cells, without the need for single cell FACS. Moreover, the latter tag enabled isolation of a pure EV population from these corrected cells to be isolated in a 1-2 hr procedure from the cell media. These EVs were detectable with luminescence if the reporter was fully expressed in the target cells. Our data shows that with this construct, EVs could be selected out of a heterogeneous pool of EVs that contained RNA solely expressed in the corrected cell. The latter observation allows the EVs derived from corrected cells to report on RNA derived from CRISPR/Cas9 events without the need for cell lysis and gene sequencing.

Two exemplary CD63 constructs were produced that enable easy (e.g., a two-step procedure) isolation and detection of EVs derived from the media/biofluids of a low number of cells (approx 10e3-10e4 cells). The CD63 construct has been shown to work in vivo (with GL261 glioma tumor cells in mice) and in vitro. When glioma cells with the CD63-reporter were injected in the brain of mice, the EVs released from the implanted tumor were trackable in mouse biofluids. Injecting EVs containing these construct were trackable in a mouse using the bioluminscence tag even in difficult to reach organs such as the brain of mice. gRNA targeting sequences against a stop codon in the genome can easily be implemented in the CD63 construct to detect CRISPR/Cas9 events altering a stop codon. If an mRNA coding sequence is targeted in the genome of the reporter cell, the affected mRNA post-gene editing can be retrieved in the reporter EVs.

Thus, a CD63 transgenic reporter protein contained in the membrane of cells and EVs can be used to detect and select out correctly gene edited EV-donor cells early on, reducing effort in avoiding cells with off targets effects.

### Exemplary Sequences:

NoMi sequence to isolate EVs and cells expressing modified CD63 construct:

(SEQ ID NO: 9)
acgcgtgtagtcttatgcaatactcttgtagtcttgcaacatggtaacgatgAGTTAGCAACATGCCTT
ACAAGGAGAGAAAAAGCACCGTGCATGCCGATTGGTGGAAGTAAGGTGGTACGATCGTGCCTTATTAGG
AAGGCAACAGACGGGTCTGACATGGATTGGACGAACCACTGAATTGCCGCATTGCAGAGATATTGTATT
TAAGTGCCTAGCTCGATACAATAAACGGGTCTCTCTGGTTAGACCAGATCTGAGCCTGGGAGCTCTCTG
GCTAACTAGGGAACCCACTGCTTAAGCCTCAATAAAGCTTGCCTTGAGTGCTTCAAGTAGTGTGTGCCC
GTCTGTTGTGTGACTCTGGTAACTAGAGATCCCTCAGACCCTTTTAGTCAGTGTGGAAAATCTCTAGCA
GTGGCGCCCGAACAGGGACTTGAAAGCGAAAGGGAAACCAGAGGAGCTCTCTCGACGCAGGACTCGGCT
TGCTGAAGCGCGCACGGCAAGAGGCGAGGGGCGGCGACTGGTGAGTACGCCAAAAATTTTGACTAGCGG
AGGCTAGAAGGAGAGAGATGGGTGCGAGAGCGTCAGTATTAAGCGGGGGAGAATTAGATCGCGATGGGA
AAAAATTCGGTTAAGGCCAGGGGGAAAGAAAAAATATAAATTAAAACATATAGTATGGGCAAGCAGGGA
GCTAGAACGATTCGCAGTTAATCCTGGCCTGTTAGAAACATCAGAAGGCTGTAGACAAATACTGGGACA
GCTACAACCATCCCTTCAGACAGGATCAGAAGAACTTAGATCATTATATAATACAGTAGCAACCCTCTA
TTGTGTGCATCAAAGGATAGAGATAAAAGACACCAAGGAAGCTTTAGACAAGATAGAGGAAGAGCAAAA
CAAAAGTAAGACCACCGCACAGCAAGCGGCCACTGATCTTCAGACCTGGAGGAGGAGATATGAGGGACA
ATTGGAGAAGTGAATTATATAAATATAAAGTAGTAAAAATTGAACCATTAGGAGTAGCACCCACCAAGG
CAAAGAGAAGAGTGGTGCAGAGAGAAAAAAGAGCAGTGGGAATAGGAGCTTTGTTCCTTGGGTTCTTGG
GAGCAGCAGGAAGCACTATGGGCGCAGCGTCAATGACGCTGACGGTACAGGCCAGACAATTATTGTCTG
GTATAGTGCAGCAGCAGAACAATTTGCTGAGGGCTATTGAGGCGCAACAGCATCTGTTGCAACTCACAG
TCTGGGGCATCAAGCAGCTCCAGGCAAGAATCCTGGCTGTGGAAAGATACCTAAAGGATCAACAGCTCC
TGGGGATTTGGGGTTGCTCTGGAAAACTCATTTGCACCACTGCTGTGCCTTGGAATGCTAGTTGGAGTA
ATAAATCTCTGGAACAGATTTGGAATCACACGACCTGGATGGAGTGGGACAGAGAAATTAACAATTACA
CAAGCTTAATACACTCCTTAATTGAAGAATCGCAAAACCAGCAAGAAAAGAATGAACAAGAATTATTGG
AATTAGATAAATGGGCAAGTTTGTGGAATTGGTTTAACATAACAAATTGGCTGTGGTATATAAAATTAT
TCATAATGATAGTAGGAGGCTTGGTAGGTTTAAGAATAGTTTTTGCTGTACTTTCTATAGTGAATAGAG
TTAGGCAGGGATATTCACCATTATCGTTTCAGACCCACCTCCCAACCCCGAGGGGACCCGACAGGCCCG
AAGGAATAGAAGAAGAAGGTGGAGAGAGAGACAGAGACAGATCCATTCGATTAGTGAACGGATCTCGAC
GGTATCGGTTAACTTTTAAAAGAAAAGGGGGGATTGGGGGGTACAGTGCAGGGGAAAGAATAGTAGACA
TAATAGCAACAGACATACAAACTAAAGAATTACAAAAACAAATTACAAAATTCAAAATTTTATCGATAC
TAGTATTATGCCCAGTACATGACCTTATGGGACTTTCCTACTTGGCAGTACATCTACGTATTAGTCATC
GCTATTACCATGGTGATGCGGTTTTGGCAGTACATCAATGGGCGTGGATAGCGGTTTGACTCACGGGGA
TTTCCAAGTCTCCACCCCATTGACGTCAATGGGAGTTTGTTTTGGCACCAAAATCAACGGGACTTTCCA
AAATGTCGTAACAACTCCGCCCCATTGACGCAAATGGGCGGTAGGCGTGTACGGTGGGAGGTTTATATA
AGCAGAGCTCGTTTAGTGAACCGTCAGATCGCCTGGAGACGCCATCCACGCTGTTTTGACCTCCATAGA
AGATTGCGGCCGCATGGCGGTGGAAGGAGGAATGAAATGTGTGAAGTTCTTGCTCTACGTCCTCCTGCT
GGCCTTTTGCGCCTGTGCAGTGGGACTGATTGCCGTGGGTGTCGGGGCACAGCTTGTCCTGAGTCAGAC
CATATCTAGAGACTACAAAGACCATGACGGTGATTATAAAGATCATGACATCGATTACAAGGATGACGA
TGACAAGGGTGGTTCTGGTGGTGGTTCTGGTCGATCCACCATGGTCTTCACACTCGAAGATTTCGTTGG
GGACTGGCGACAGACAGCCGGCTACAACCTGGACCAAGTCCTTGAACAGGGAGGTGTGTCCAGTTTGTT
TCAGAATCTCGGGGTGTCCGTAACTCCGATCCAAAGGATTGTCCTGAGCGGTGAAAATGGGCTGAAGAT
CGACATCCATGTCATCATCCCGTATGAAGGTCTGAGCGGCGACCAAATGGGCCAGATCGAAAAAATTTT
TAAGGTGGTGTACCCTGTGGATGATCATCACTTTAAGGTGATCCTGCACTATGGCACACTGGTAATCGA
CGGGGTTACGCCGAACATGATCGACTATTTCGGACGGCCGTATGAAGGCATCGCCGTGTTCGACGGCAA
AAAGATCACTGTAACAGGGACCCTGTGGAACGGCAACAAAATTATCGACGAGCGCCTGATCAACCCCGA
CGGCTCCCTGCTGTTCCGAGTAACCATCAACGGAGTGACCGGCTGGCGGCTGTGCGAACGCATTCTGGC
GTCTAGAATCCAGGGGGCTACCCCTGGCTCTCTGTTGCCAGTGGTCATCATCGCAGTGGGTGTCTTCCT
CTTCCTGGTGGCTTTTGTGGGCTGCTGCGGGGCCTGCAAGGAGAACTATTGTCTTATGATCACGTTTGC
CATCTTTCTGTCTCTTATCATGTTGGTGGAGGTGGCCGCAGCCATTGCTGGCTATGTGTTTAGAGATAA
GGTGATGTCAGAGTTTAATAACAACTTCCGGCAGCAGATGGAGAATTACCCGAAAAATAACCACACTGC
TTCGATCCTGGACAGGATGCAGGCAGATTTTAAGTGCTGTGGGGCTGCTAACTACACAGATTGGGAGAA
AATCCCTTCCATGTCGAAGAACCGAGTCCCCGACTCCTGCTGCATTAATGTTACTGTGGGCTGTGGGAT
TAATTTCAACGAGAAGGCGATCCATAAGGAGGGCTGTGTGGAGAAGATTGGGGGCTGGCTGAGGAAAAA
TGTGCTGGTGGTAGCTGCAGCAGCCCTTGGAATTGCTTTTGTCGAGGTTTTGGGAATTGTCTTTGCCTG
CTGCCTTGTGAAGAGTATCAGAAGTGGCTACGAGGTGATGGAATTCGCTAGCGCTACCGGACTCAGATC
TCGAGCTCAAGCTTCGAATTCTGCAGTCGACGGTACCGCGGGCCCGGGATCCACCGGTATGGTGAGCAA
GGGCGAGGAGGATAACATGGCCATCATCAAGGAGTTCATGCGCTTCAAGGTGCACATGGAGGGCTCCGT
GAACGGCCACGAGTTCGAGATCGAGGGCGAGGGCGAGGGCCGCCCCTACGAGGGCACCCAGACCGCCAA
GCTGAAGGTGACCAAGGGTGGCCCCCTGCCCTTCGCCTGGGACATCCTGTCCCCTCAGTTCATGTACGG
CTCCAAGGCCTACGTGAAGCACCCCGCCGACATCCCCGACTACTTGAAGCTGTCCTTCCCCGAGGGCTT
CAAGTGGGAGCGCGTGATGAACTTCGAGGACGGCGGCGTGGTGACCGTGACCCAGGACTCCTCCCTGCA
GGACGGCGAGTTCATCTACAAGGTGAAGCTGCGCGGCACCAACTTCCCCTCCGACGGCCCCGTAATGCA
GAAGAAGACCATGGGCTGGGAGGCCTCCTCCGAGCGGATGTACCCCGAGGACGGCGCCCTGAAGGGCGA
GATCAAGCAGAGGCTGAAGCTGAAGGACGGCGGCCACTACGACGCTGAGGTCAAGACCACCTACAAGGC
CAAGAAGCCCGTGCAGCTGCCCGGCGCCTACAACGTCAACATCAAGTTGGACATCACCTCCCACAACGA
GGACTACACCATCGTGGAACAGTACGAACGCGCCGAGGGCCGCCACTCCACCGGCGGCATGGACGAGCT
GTACAAGGCTAGCGCTACCGGACTCAGATCTCGAGCTCAAGCTTCGAATTCTGCAGTCGACGGTACCGC
GGGCCCGGGATCCACCGGTAAGCTTGGAAGCGGAGCTACTAACTTCAGCCTGCTGAAGCAGGCTGGAGA
CGTGGAGGAGAACCCTGGACCTATGGAGAGCGACGAGAGCGGCCTGCCCGCCATGGAGATCGAGTGCCG
CATCACCGGCACCCTGAACGGCGTGGAGTTCGAGCTGGTGGGCGGCGGAGAGGGCACCCCCAAGCAGGG
CCGCATGACCAACAAGATGAAGAGCACCAAAGGCGCCCTGACCTTCAGCCCCTACCTGCTGAGCCACGT
GATGGGCTACGGCTTCTACCACTTCGGCACCTACCCCAGCGGCTACGAGAACCCCTTCCTGCACGCCAT
CAACAACGGCGGCTACACCAACACCCGCATCGAGAAGTACGAGGACGGCGGCGTGCTGCACGTGAGCTT
CAGCTACCGCTACGAGGCCGGCCGCGTGATCGGCGACTTCAAGGTGGTGGGCACCGGCTTCCCCGAGGA
CAGCGTGATCTTCACCGACAAGATCATCCGCAGCAACGCCACCGTGGAGCACCTGCACCCCATGGGCGA
TAACGTGCTGGTGGGCAGCTTCGCCCGCACCTTCAGCCTGCGCGACGGCGGCTACTACAGCTTCGTGGT
GGACAGCCACATGCACTTCAAGAGCGCCATCCACCCCAGCATCCTGCAGAACGGGGGCCCCATGTTCGC
CTTCCGCCGCGTGGAGGAGCTGCACAGCAACACCGAGCTGGGCATCGTGGAGTACCAGCACGCCTTCAA
GACCCCCATTGCCTTCGCCAGATCCCGCGCTCAGTCGTCCAATTCTGCCGTGGACGGCACCGCCGGACC
CGGCTCCACCGGATCTCGCGTCGACGGCAGTGGAGAGGGCAGAGGAAGTCTGCTAACATGCGGTGACGT
CGAGGAGAATCCTGGCCCAATGACCGAGTACAAGCCCACGGTGCGCCTCGCCACCCGCGACGACGTCCC
CAGGGCCGTACGCACCCTCGCCGCCGCGTTCGCCGACTACCCCGCCACGCGCCACACCGTCGATCCGGA
CCGCCACATCGAGCGGGTCACCGAGCTGCAAGAACTCTTCCTCACGCGCGTCGGGCTCGACATCGGCAA
GGTGTGGGTCGCGGACGACGGCGCCGCGGTGGCGGTCTGGACCACGCCGGAGAGCGTCGAAGCGGGGGC
GGTGTTCGCCGAGATCGGCCCGCGCATGGCCGAGTTGAGCGGTTCCCGGCTGGCCGCGCAGCAACAGAT
GGAAGGCCTCCTGGCGCCGCACCGGCCCAAGGAGCCCGCGTGGTTCCTGGCCACCGTCGGCGTCTCGCC
CGACCACCAGGGCAAGGGTCTGGGCAGCGCCGTCGTGCTCCCCGGAGTGGAGGCGGCCGAGCGCGCCGG
GGTGCCCGCCTTCCTGGAGACCTCCGCGCCCCGCAACCTCCCCTTCTACGAGCGGCTCGGCTTCACCGT
CACCGCCGACGTCGAGGTGCCCGAAGGACCGCGCACCTGGTGCATGACCCGCAAGCCCGGTGCCTGAGA
ATTCGTCGACAATCAACCTCTGGATTACAAAATTTGTGAAAGATTGACTGGTATTCTTAACTATGTTGC
TCCTTTTACGCTATGTGGATACGCTGCTTTAATGCCTTTGTATCATGCTATTGCTTCCCGTATGGCTTT
CATTTTCTCCTCCTTGTATAAATCCTGGTTGCTGTCTCTTTATGAGGAGTTGTGGCCCGTTGTCAGGCA
ACGTGGCGTGGTGTGCACTGTGTTTGCTGACGCAACCCCCACTGGTTGGGGCATTGCCACCACCTGTCA
GCTCCTTTCCGGGACTTTCGCTTTCCCCCTCCCTATTGCCACGGCGGAACTCATCGCCGCCTGCCTTGC
CCGCTGCTGGACAGGGGCTCGGCTGTTGGGCACTGACAATTCCGTGGTGTTGTCGGGGAAATCATCGTC
CTTTCCTTGGCTGCTCGCCTGTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGCCTGGTACCTTTAAGACCAATGA
CTTACAAGGCAGCTGTAGATCTTAGCCACTTTTTAAAAGAAAAGGGGGGACTGGAAGGGCTAATTCACT
CCCAACGAAGATAAGATCTGCTTTTTGCTTGTACTGGGTCTCTCTGGTTAGACCAGATCTGAGCCTGGG
AGCTCTCTGGCTAACTAGGGAACCCACTGCTTAAGCCTCAATAAAGCTTGCCTTGAGTGCTTCAAGTAG
TGTGTGCCCGTCTGTTGTGTGACTCTGGTAACTAGAGATCCCTCAGACCCTTTTAGTCAGTGTGGAAAA
TCTCTAGCAGTAGTAGTTCATGTCATCTTATTATTCAGTATTTATAACTTGCAAAGAAATGAATATCAG
AGAGTGAGAGGAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCTTATCATG
TCTGGCTCTAGCTATCCCGCCCCTAACTCCGCCCATCCCGCCCCTAACTCCGCCCAGTTCCGCCCATTC
TCCGCCCCATGGCTGACTAATTTTTTTTATTTATGCAGAGGCCGAGGCCGCCTCGGCCTCTGAGCTATT
CCAGAAGTAGTGAGGAGGCTTTTTTGGAGGCCTAGACTTTTGCAGAGACCAAATTCGTAATCATGTCAT
AGCTGTTTCCTGTGTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCACTGCCCGCTTTCC
AGTCGGGAAACCTGTCGTGCCAGCTGCATTAATGAATCGGCCAACGCGCGGGGAGAGGCGGTTTGCGTA
TTGGGCGCTCTTCCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGAAAGAACATGTGAG
CAAAAGGCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGCCTTAT
CCGGTAACTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAACTACGGCT
ACACTAGAAGAACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAACGAAA
ACTCACGTTAAGGGATTTTGGTCATGAGATTATCAAAAAGGATCTTCACCTAGATCCTTTTAAATTAAA
AATGAAGTTTTAAATCAATCTAAAGTATATATGAGTAAACTTGGTCTGACAGTTACCAATGCTTAATCA
GTGAGGCACCTATCTCAGCGATCTGTCTATTTCGTTCATCCATAGTTGCCTGACTCCCCGTCGTGTAGA
TAACTACGATACGGGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAGTGGTCCTGCAACTT
TATCCGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTCATTCA
GCTCCGGTTCCCAACGATCAAGGCGAGTTACATGATCCCCCATGTTGTGCAAAAAAGCGGTTAGCTCCT
TCGGTCCTCCGATCGTTGTCAGAAGTAAGTTGGCCGCAGTGTTATCACTCATGGTTATGGCAGCACTGC
ATAATTCTCTTACTGTCATGCCATCCGTAAGATGCTTTTCTGTGACTGGTGAGTACTCAACCAAGTCAT
TCTGAGAATAGTGTATGCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAACTCTCAAGGATCTTAC
CGCTGTTGAGATCCAGTTCGATGTAACCCACTCGTGCACCCAACTGATCTTCAGCATCTTTTACTTTCA
CCAGCGTTTCTGGGTGAGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGTTATTGTCTCATGA
GCGGATACATATTTGAATGTATTTAGAAAAATAAACAAATAGGGGTTCCGCGCACATTTCCCCGAAAAG
TGCCACCTGACGTCTAAGAAACCATTATTATCATGACATTAACCTATAAAAATAGGCGTATCACGAGGC
CCTTTCGTCTCGCGCGTTTCGGTGATGACGGTGAAAACCTCTGACACATGCAGCTCCCGGAGACGGTCA
CAGCTTGTCTGTAAGCGGATGCCGGGAGCAGACAAGCCCGTCAGGGCGCGTCAGCGGGTGTTGGCGGGT
GTCGGGGCTGGCTTAACTATGCGGCATCAGAGCAGATTGTACTGAGAGTGCACCATATGCGGTGTGAAA
TACCGCACAGATGCGTAAGGAGAAAATACCGCATCAGGCGCCATTCGCCATTCAGGCTGCGCAACTGTT
GGGAAGGGCGATCGGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCAGTGCCAAGCTGa
cgcgtgtagtcttatgcaatactcttgtagtcttgcaacatggtaacgatg

Example sequence of CRISPR reporter lentiviral vector:

(SEQ ID NO: 10)
ccaagtcattctgagaatagtgtatgcggcgaccgagttgctcttgcccggcGTCAATACGGGATAATA
CCGCGCCACATAGCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGATCTTCAGCATCTT
TTACTTTCACCAGCGTTTCTGGGTGAGCAAAAACAGGAAGGCAAAATGCCGCAAAAAAGGGAATAAGGG
CGACACGGAAATGTTGAATACTCATACTCTTCCTTTTTCAATATTATTGAAGCATTTATCAGGGTTATT
GTCTCATGAGCGGATACATATTTGAATGTATTTAGAAAAATAAACAAATAGGGGTTCCGCGCACATTTC
CCCGAAAAGTGCCACCTGACGTCGACGGATCGGGAGATCTCCCGATCCCCTATGGTGCACTCTCAGTAC
AATCTGCTCTGATGCCGCATAGTTAAGCCAGTATCTGCTCCCTGCTTGTGTGTTGGAGGTCGCTGAGTA
GTGCGCGAGCAAAATTTAAGCTACAACAAGGCAAGGCTTGACCGACAATTGCATGAAGAATCTGCTTAG
GGTTAGGCGTTTTGCGCTGCTTCGCGATGTACGGGCCAGATATACGCGTTGACATTGATTATTGACTAG
TTATTAATAGTAATCAATTACGGGGTCATTAGTTCATAGCCCATATATGGAGTTCCGCGTTACATAACT
TACGGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACATCTACGTATTAGT
CATCGCTATTACCATGGTGATGCGGTTTTGGCAGTACATCAATGGGCGTGGATAGCGGTTTGACTCACG
GGGATTTCCAAGTCTCCACCCCATTGACGTCAATGGGAGTTTGTTTTGGCACCAAAATCAACGGGACTT
TCCAAAATGTCGTAACAACTCCGCCCCATTGACGCAAATGGGCGGTAGGCGTGTACGGTGGGAGGTCTA
TATAAGCAGCGCGTTTTGCCTGTACTGGGTCTCTCTGGTTAGACCAGATCTGAGCCTGGGAGCTCTCTG
GCTAACTAGGGAACCCACTGCTTAAGCCTCAATAAAGCTTGCCTTGAGTGCTTCAAGTAGTGTGTGCCC
GTCTGTTGTGTGACTCTGGTAACTAGAGATCCCTCAGACCCTTTTAGTCAGTGTGGAAAATCTCTAGCA
GTGGCGCCCGAACAGGGACTTGAAAGCGAAAGGGAAACCAGAGGAGCTCTCTCGACGCAGGACTCGGCT
TGCTGAAGCGCGCACGGCAAGAGGCGAGGGGCGGCGACTGGTGAGTACGCCAAAAATTTTGACTAGCGG
AGGCTAGAAGGAGAGAGATGGGTGCGAGAGCGTCAGTATTAAGCGGGGGAGAATTAGATCGCGATGGGA
AAAAATTCGGTTAAGGCCAGGGGGAAAGAAAAAATATAAATTAAAACATATAGTATGGGCAAGCAGGGA
GCTAGAACGATTCGCAGTTAATCCTGGCCTGTTAGAAACATCAGAAGGCTGTAGACAAATACTGGGACA
GCTACAACCATCCCTTCAGACAGGATCAGAAGAACTTAGATCATTATATAATACAGTAGCAACCCTCTA
TTGTGTGCATCAAAGGATAGAGATAAAAGACACCAAGGAAGCTTTAGACAAGATAGAGGAAGAGCAAAA
CAAAAGTAAGACCACCGCACAGCAAGCGGCCGGCCGCTGATCTTCAGACCTGGAGGAGGAGATATGAGG
GACAATTGGAGAAGTGAATTATATAAATATAAAGTAGTAAAAATTGAACCATTAGGAGTAGCACCCACC
AAGGCAAAGAGAAGAGTGGTGCAGAGAGAAAAAAGAGCAGTGGGAATAGGAGCTTTGTTCCTTGGGTTC
TTGGGAGCAGCAGGAAGCACTATGGGCGCAGCGTCAATGACGCTGACGGTACAGGCCAGACAATTATTG
TCTGGTATAGTGCAGCAGCAGAACAATTTGCTGAGGGCTATTGAGGCGCAACAGCATCTGTTGCAACTC
ACAGTCTGGGGCATCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGGAATGCTAGTTGG
AGTAATAAATCTCTGGAACAGATTTGGAATCACACGACCTGGATGGAGTGGGACAGAGAAATTAACAAT
TACACAAGCTTAATACACTCCTTAATTGAAGAATCGCAAAACCAGCAAGAAAAGAATGAACAAGAATTA
TTGGAATTAGATAAATGGGCAAGTTTGTGGAATTGGTTTAACATAACAAATTGGCTGTGGTATATAAAA
TTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGG
CCCGAAGGAATAGAAGAAGAAGGTGGAGAGAGAGACAGAGACAGATCCATTCGATTAGTGAACGGATCG
GCACTGCGTGCGCCAATTCTGCAGACAAATGGCAGTATTCATCCACAATTTTAAAAGAAAAGGGGGGAT
TGGGGGGTACAGTGCAGGGGAAAGAATAGTAGACATAATAGCAACAGACATACAAACTAAAGAATTACA
AAAACAAATTACAAAAATTCAAAATTTTCGGGTTTATTACAGGGACAGCAGAGATCCAGTTTGGTTAGT
ACCGGGCCCGCTCTAGCGTGAGGCTCCGGTGCCCGTCAGTGGGCAGAGCGCACATCGCCCACAGTCCCC
GAGAAGTTGGGGGGAGGGGTCGGCAATTGAACCGGTGCCTAGAGAAGGTGGCGCGGGGTAAACTGGGAA
AGTGATGTCGTGTACTGGCTCCGCCTTTTTCCCGAGGGTGGGGGAGAACCGTATATAAGTGCAGTAGTC
GCCGTGAACGTTCTTTTTCGCAACGGGTTTGCCGCCAGAACACAGGTAAGTGCCGTGTGTGGTTCCCGC
GGGCCTGGCCTCTTTACGGGTTATGGCCCTTGCGTGCCTTGAATTACTTCCACCTGGCTGCAGTACGTG
ATTCTTGATCCCGAGCTTCGGGTTGGAAGTGGGTGGGAGAGTTCGAGGCCTTGCGCTTAAGGAGCCCCT
TCGCCTCGTGCTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGCTGCGACGCTT
TTTTTCTGGCAAGATAGTCTTGTAAATGCGGGCCAAGATCTGCACACTGGTATTTCGGTTTTTGGGGCC
GCGGGCGGCGACGGGGCCCGTGCGTCCCAGCGCACATGTTCGGCGAGGCGGGGCCTGCGAGCGCGGCCA
CCGAGAATCGGACGGGGGTAGTCTCAAGCTGGCCGGCCTGCTCTGGTGCCTGGCCTCGCGCCGCCGTGT
ATCGCCCCGCCCTGGGCGGCAAGGCTGGCCCGGTCGGCACCAGTTGCGTGAGCGGAAAGATGGCCGCTT
CCCGGCCCTGCTGCAGGGAGCTCAAAATGGAGGACGCGGCGCTCGGGAGAGCGGGCGGGTGAGTCACCC
ACACAAAGGAAAAGGGCCTTTCCGTCCTCAGCCGTCGCTTCATGTGACTCCACGGAGTACCGGGCGCCG
TCCAGGCACCTCGATTAGTTCTCGAGCTTTTGGAGTACGTCGTCTTTAGGTTGGGGGGAGGGGTTTTAT
GCGATGGAGTTTCCCCACACTGAGTGGGTGGAGACTGAAGTTAGGCCAGCTTGGCACTTGATGTAATTC
TCCTTGGAATTTGCCCTTTTTGAGTTTGGATCTTGGTTCATTCTCAAGCCTCAGACAGTGGTTCAAAGT
TTTTTTCTTCCATTTCAGGTGTCGTGAGCGGCCGCTGAGTTAACTATTATGGCGGTGGAAGGAGGAATG
AAATGTGTGAAGTTCTTGCTCTACGTCCTCCTGCTGGCCTTTTGCGCCTGTGCAGTGGGACTGATTGCC
GTGGGTGTCGGGGCACAGCTTGTCCTGAGTCAGACCTAAAAGGTCATACCCATACGATGTTCCTGACTA
TGCGGGCTATCCCTATGACGTCCCGGACTATGCAGGATCCTATCCATATGACGTTCCAGATTACGCTGT
TAACTCTGGTGTAGCAGGTCATGCCTCTGGCAGCCCCGCATTCGGGACCGCCTCTCATTCGAATTGCGA
ACATGAAGAGATCCACCTCGCCGGCTCGATCCAGCCGCATGGCGCGCTTCTGGTCGTCAGCGAACATGA
TCATCGCGTCATCCAGGCCAGCGCCAACGCCGCGGAATTTCTGAATCTCGGAAGCGTACTCGGCGTTCC
GCTCGCCGAGATCGACGGCGATCTGTTGATCAAGATCCTGCCGCATTTAGATCCAACCGCAGAAGGCAT
GCCGGTCGCGGTGCGCTGCCGGATCGGTAATCCCTCTACGGAGTACTGCGGTCTGATGCATCGGCCTCC
GGAAGGCGGTCTGATCATCGAACTCGAACGTGCAGGACCATCGATCGATCTGTCAGGCACGCTGGCGCC
GGCGTTAGAGCGGATCCGCACGGCGGGTTCACTGCGCGCACTGTGTGATGACACAGTACTGCTGTTTCA
GCAGTGCACCGGCTACGACCGGGTGATGGTGTATCGTTTCGATGAGCAAGGCCACGGATTAGTATTCTC
CGAGTGCCATGTGCCTGGGCTCGAATCCTATTTCGGCAACCGCTATCCGTCGTCGCTGGTCCCGCAGAT
GGCAAGACAGCTGTACGTGAGACAGCGCGTCCGCGTGCTGGTCGACGTAACTTATCAACCAGTGCCGCT
GGAGCCACGGCTGTCGCCGCTGACTGGAAGAGATCTCGACATGTCGGGCTGCTTCCTGCGCTCGATGTC
GCCGATCCATTTACAGTTCCTGAAGGACATGGGCGTGCGCGCCACCCTAGCAGTTTCACTGGTGGTCGG
CGGCAAGCTGTGGGGCCTGGTTGTCTGTCACCATTATCTGCCGCGCTTCATCCGTTTCGAGTTACGGGC
GATCTGCAAACGGCTCGCCGAAAGGATCGCGACGAGAATAACCGCACTTGAATCCATCCAGGGGGCTAC
CCCTGGCTCTCTGTTGCCAGTGGTCATCATCGCAGTGGGTGTCTTCCTCTTCCTGGTGGCTTTTGTGGG
CTGCTGCGGGGCCTGCAAGGAGAACTATTGTCTTATGATCACGTTTGCCATCTTTCTGTCTCTTATCAT
GTTGGTGGAGGTGGCCGCAGCCATTGCTGGCTATGTGTTTAGAGATAAGGTGATGTCAGAGTTTAATAA
CAACTTCCGGCAGCAGATGGAGAATTACCCGAAAAATAACCACACTGCTTCGATCCTGGACAGGATGCA
GGCAGATTTTAAGTGCTGTGGGGCTGCTAACTACACAGATTGGGAGAAAATCCCTTCCATGTCGAAGAA
CCGAGTCCCCGACTCCTGCTGCATTAATGTTACTGTGGGCTGTGGGATTAATTTCAACGAGAAGGCGAT
CCATAAGGAGGGCTGTGTGGAGAAGATTGGGGGCTGGCTGAGGAAAAATGTGCTGGTGGTAGCTGCAGC
AGCCCTTGGAATTGCTTTTGTCGAGGTTTTGGGAATTGTCTTTGCCTGCTGCCTCGTGAAGAGTATCAG
AAGTGGCTACGAGGTGATGGAATTCATGGAAGACGCCAAAAACATAAAGAAAGGCCCGGCGCCATTCTA
TCCGCTGGAAGATGGAACCGCTGGAGAGCAACTGCATAAGGCTATGAAGAGATACGCCCTGGTTCCTGG
AACAATTGCTTTTACAGATGCACATATCGAGGTGGACATCACTTACGCTGAGTACTTCGAAATGTCCGT
TCGGTTGGCAGAAGCTATGAAACGATATGGGCTGAATACAAATCACAGAATCGTCGTATGCAGTGAAAA
CTCTCTTCAATTCTTTATGCCGGTGTTGGGCGCGTTATTTATCGGAGTTGCAGTTGCGCCCGCGAACGA
CATTTATAATGAACGTGAATTGCTCAACAGTATGGGCATTTCGCAGCCTACCGTGGTGTTCGTTTCCAA
AAAGGGGTTGCAAAAAATTTTGAACGTGCAAAAAAAGCTCCCAATCATCCAAAAAATTATTATCATGGA
TTCTAAAACGGATTACCAGGGATTTCAGTCGATGTACACGTTCGTCACATCTCATCTACCTCCCGGTTT
TAATGAATACGATTTTGTGCCAGAGTCCTTCGATAGGGACAAGACAATTGCACTGATCATGAACTCCTC
TGGATCTACTGGTCTGCCTAAAGGTGTCGCTCTGCCTCATAGAACTGCCTGCGTGAGATTCTCGCATGC
CAGAGATCCTATTTTTGGCAATCAAATCATTCCGGATACTGCGATTTTAAGTGTTGTTCCATTCCATCA
CGGTTTTGGAATGTTTACTACACTCGGATATTTGATATGTGGATTTCGAGTCGTCTTAATGTATAGATT
TGAAGAAGAGCTGTTTCTGAGGAGCCTTCAGGATTACAAGATTCAAAGTGCGCTGCTGGTGCCAACCCT
ATTCTCCTTCTTCGCCAAAAGCACTCTGATTGACAAATACGATTTATCTAATTTACACGAAATTGCTTC
TGGTGGCGCTCCCCTCTCTAAGGAAGTCGGGGAAGCGGTTGCCAAGAGGTTCCATCTGCCAGGTATCAG
GCAAGGATATGGGCTCACTGAGACTACATCAGCTATTCTGATTACACCCGAGGGGGATGATAAACCGGG
CGCGGTCGGTAAAGTTGTTCCATTTTTTGAAGCGAAGGTTGTGGATCTGGATACCGGGAAAACGCTGGG
CGTTAATCAAAGAGGCGAACTGTGTGTGAGAGGTCCTATGATTATGTCCGGTTATGTAAACAATCCGGA
AGCGACCAACGCCTTGATTGACAAGGATGGATGGCTACATTCTGGAGACATAGCTTACTGGGACGAAGA
CGAACACTTCTTCATCGTTGACCGCCTGAAGTCTCTGATTAAGTACAAAGGCTATCAGGTGGCTCCCGC
TGAATTGGAATCCATCTTGCTCCAACACCCCAACATCTTCGACGCAGGTGTCGCAGGTCTTCCCGACGA
TGACGCCGGTGAACTTCCCGCCGCCGTTGTTGTTTTGGAGCACGGAAAGACGATGACGGAAAAAGAGAT
CGTGGATTACGTCGCCAGTCAAGTAACAACCGCGAAAAAGTTGCGCGGAGGAGTTGTGTTTGTGGACGA
AGTACCGAAAGGTCTTACCGGAAAACTCGACGCAAGAAAAATCAGAGAGATCCTCATAAAGGCCAAGAA
GGGCGGAAAGATCGCCGTGCTAGAGTACCCGGGCTAGGATCCGCCCCTCTCCCTCCCCCCCCCCTAACG
TTACTGGCCGAAGCCGCTTGGAATAAGGCCGGTGTGCGTTTGTCTATATGTTATTTTCCACCATATTGC
CGTCTTTTGGCAATGTGAGGGCCCGGAAACCTGGCCCTGTCTTCTTGACGAGCATTCCTAGGGGTCTTT
CCCCTCTCGCCAAAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGCGACAGGTGCCTCT
GCGGCCAAAAGCCACGTGTATAAGATACACCTGCAAAGGCGGCACAACCCCAGTGCCACGTTGTGAGTT
GGATAGTTGTGGAAAGAGTCAAATGGCTCTCCTCAAGCGTATTCAACAAGGGGCTGAAGGATGCCCAGA
AGGTACCCCATTGTATGGGATCTGATCTGGGGCCTCGGTACACATGCTTTACATGTGTTTAGTCGAGGT
TAAAAAAACGTCTAGGCCCCCCGAACCACGGGGACGTGGTTTTCCTTTGAAAAACACGATGATAATATG
GCCACAACCATGTCACCAGAGCCAGCGAAGTCTGCTCCCGCCCCGAAAAAGGGCTCCAAGAAGGCGGTG
ACTAAGGCGCAGAAGAAAGGCGGCAAGAAGCGCAAGCGCAGCCGCAAGGAGAGCTATTCCATCTATGTG
TACAAGGTTCTGAAGCAGGTCCACCCTGACACCGGCATTTCGTCCAAGGCCATGGGCATCATGAATTCG
TTTGTGAACGACATTTTCGAGCGCATCGCAGGTGAGGCTTCCCGCCTGGCGCATTACAACAAGCGCTCG
ACCATCACCTCCAGGGAGATCCAGACGGCCGTGCGCCTGCTGCTGCCTGGGGAGTTGGCCAAGCACGCC
GTGTCCGAGGGTACTAAGGCCATCACCAAGTACACCAGCGCTAAGGATCCACCGGTCGCCACCATGGTG
AGCAAGGGCGAGGAGCTGTTCACCGGGGTGGTGCCCATCCTGGTCGAGCTGGACGGCGACGTAAACGGC
CACAAGTTCAGCGTGTCCGGCGAGGGCGAGGGCGATGCCACCTACGGCAAGCTGACCCTGAAGTTCATC
TGCACCACCGGCAAGCTGCCCGTGCCCTGGCCCACCCTCGTGACCACCCTGACCTACGGCGTGCAGTGC
TTCAGCCGCTACCCCGACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGGGGCAC
AAGCTGGAGTACAACTACAACAGCCACAACGTCTATATCATGGCCGACAAGCAGAAGAACGGCATCAAG
GTGAACTTCAAGATCCGCCACAACATCGAGGACGGCAGCGTGCAGCTCGCCGACCACTACCAGCAGAAC
ACCCCCATCGGCGACGGCCCCGTGCTGCTGCCCGACAACCACTACCTGAGCACCCAGTCCGCCCTGAGC
AAAGACCCCAACGAGAAGCGCGATCACATGGTCCTGCTGGAGTTCGTGACCGCCGCCGGGATCACTCTC
GGCATGGACGAGCTGTACAAGTAAAGCGGCCGCGACATGACATCGATACCGTCGACCTCGATCGAGACC
TAGAAAAACATGGAGCAATCACAAGTAGCAATACAGCAGCTACCAATGCTGATTGTGCCTGGCTAGAAG
CACAAGAGGAGGAGGAGGTGGGTTTTCCAGTCACACCTCAGGTACCTTTAAGACCAATGACTTACAAGG
CAGCTGTAGATCTTAGCCACTTTTTAAAAGAAAAGGGGGGACTGGAAGGGCTAATTCACTCCCAACGAA
GACAAGATATCCTTGATCTGTGGATCTACCACACACAAGGCTACTTCCCTGATTGGCAGAACTACACAC
CAGGGCCAGGGATCAGATATCCACTGACCTTTGGATGGTGCTACAAGCTAGTACCAGTTGAGCAAGAGA
AGGTAGAAGAAGCCAATGAAGGAGAGAACACCCGCTTGTTACACCCTGTGAGCCTGCATGGGATGGATG
ACCCGGAGAGAGAAGTATTAGAGTGGAGGTTTGACAGCCGCCTAGCATTTCATCACATGGCCCGAGAGC
TGCATCCGGACTGTACTGGGTCTCTCTGGTTAGACCAGATCTGAGCCTGGGAGCTCTCTGGCTAACTAG
GGAACCCACTGCTTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCTAGCAGCATGTGAG
CAAAAGGCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGCCTTAT
CCGGTAACTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAACTACGGCT
ACACTAGAAGAACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAACGAAA
ACTCACGTTAAGGGATTTTGGTCATGAGATTATCAAAAAGGATCTTCACCTAGATCCTTTTAAATTAAA
AATGAAGTTTTAAATCAATCTAAAGTATATATGAGTAAACTTGGTCTGACAGTTACCAATGCTTAATCA
GTGAGGCACCTATCTCAGCGATCTGTCTATTTCGTTCATCCATAGTTGCCTGACTCCCCGTCGTGTAGA
TAACTACGATACGGGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAGTGGTCCTGCAACTT
TATCCGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTCATTCA
GCTCCGGTTCCCAACGATCAAGGCGAGTTACATGATCCCCCATGTTGTGCAAAAAAGCGGTTAGCTCCT
TCGGTCCTCCGATCGTTGTCAGAAGTAAGTTGGCCGCAGTGTTATCACTCATGGTTATGGCAGCACTGC
ATAATTCTCTTACTGTCATGCCATCCGTAAGATGCTTTTCTGTGACTGGTGAGTACTCAAccaagtcat
tctgagaatagtgtatgcggcgaccgagttgctcttgcccggc

### REFERENCES

1. Hoen, E. N., Cremer, T., Gallo, R. C. & Margolis, L. B. Extracellular vesicles and viruses: Are they close relatives? Proceedings of the National Academy of Sciences of the United States of America 113, 9155-9161 (2016).
2. Segel, M. et al. Mammalian retrovirus-like protein PEG10 packages its own mRNA and can be pseudotyped for mRNA delivery. Science (1979) 373, 882-889 (2021).
3. Mathieu, M., Martin-Jaular, L., Lavieu, G. & Théry, C. Specificities of secretion and uptake of exosomes and other extracellular vesicles for cell-to-cell communication. Nature Cell Biology 21, 9-17 (2019).
4. Resch, U. et al. A two-component regulatory system impacts extracellular membrane-derived vesicle production in group a Streptococcus. mBio 7, (2016).
5. Wiklander, O. P. B., Brennan, M., Lötvall, J., Breakefield, X. O. & Andaloussi, S. E. L. Advances in therapeutic applications of extracellular vesicles. Science Translational Medicine 11, 8521 (2019).
6. Murphy, D. E. et al. Extracellular vesicle-based therapeutics: natural versus engineered targeting and trafficking. Experimental and Molecular Medicine 51 1-12 (2019).
7. Banskota, S. et al. Engineered virus-like particles for efficient in vivo delivery of therapeutic proteins. Cell 185, 250-265.e16 (2022).
8. Abels, E. R. et al. Glioblastoma-Associated Microglia Reprogramming Is Mediated by Functional Transfer of Extracellular miR-21. Cell Reports 28, 3105-3119.e7 (2019).
9. O'Brien, K., Breyne, K., Ughetto, S., Laurent, L. C. & Breakefield, X. O. RNA delivery by extracellular vesicles in mammalian cells and its applications. Nature Reviews Molecular Cell Biology 21, (2020).
10. Shete, H. K., Prabhu, R. H. & Patravale, V. B. Endosomal escape: A bottleneck in intracellular delivery. Journal of Nanoscience and Nanotechnology 14 460-474 (2014).
11. Somiya, M. & Kuroda, S. Real-Time Luminescence Assay for Cytoplasmic Cargo Delivery of Extracellular Vesicles. Analytical Chemistry 93, 5612-5620 (2021).
12. Rufino-Ramos, D. et al. Extracellular vesicles: Novel promising delivery systems for therapy of brain diseases. Journal of Controlled Release 262, 247-258 (2017).
13. Nasiri Kenari, A., Cheng, L. & Hill, A. F. Methods for loading therapeutics into extracellular vesicles and generating extracellular vesicles mimetic-nanovesicles. Methods 177, 103-113 (2020).
14. Ma, C., Malessa, A., Boersma, A. J., Liu, K. & Herrmann, A. Supercharged Proteins and Polypeptides. Advanced Materials 32, 1905309 (2020).
15. McNaughton, B. R., Cronican, J. J., Thompson, D. B. & Liu, D. R. Mammalian cell penetration, siRNA transfection, and DNA transfection by supercharged proteins. Proc Natl Acad Sci U S A 106, 6111-6116 (2009).
16. Tsien, R. Y. THE GREEN FLUORESCENT PROTEIN. Annu. Rev. Biochem 67, 509-553 (1998).
17. Markwardt, M. L., Kremers, G .-J., Kraft, C. A., Ray, K. & Cranfill, P. J. C. An Improved Cerulean Fluorescent Protein with Enhanced Brightness and Reduced Reversible Photoswitching. PLOS ONE 6, 17896 (2011).
18. Lawrence, M. S., Phillips, K. J. & Liu, D. R. Supercharging proteins can impart unusual resilience. J Am Chem Soc 129, 10110-10112 (2007).
19. Kruse, T., Schneider, S., Reger, L. N., Kampmann, M. & Reif, O.-W. A novel approach for enumeration of extracellular vesicles from crude and purified cell culture samples. (2022) doi: 10.1002/elsc.202100149.
20. Fraser, K. et al. Characterization of single microvesicles in plasma from glioblastoma patients. 21, 606-615 (2018).
21. Rigaud, J. L. et al. Bio-Beads: An efficient strategy for two-dimensional crystallization of membrane proteins. Journal of Structural Biology 118, 226-235 (1997).
22. Mishin, V. P., Novikov, D., Hayden, F. G. & Gubareva, L. v. Effect of Hemagglutinin Glycosylation on Influenza Virus Susceptibility to Neuraminidase Inhibitors. Journal of Virology 79, 12416-12424 (2005).
23. Marcu, I. C. et al. Isolation of Human Small Extracellular Vesicles and Tracking of Their Uptake by Retinal Pigment Epithelial Cells In Vitro. International Journal of Molecular Sciences 21, 3799 (2020).
24. Trajkovic, K. et al. Ceramide triggers budding of exosome vesicles into multivesicular endosomes. Science (1979) 319, 1244-1247 (2008).
25. Ashfaq, U. A., Javed, T., Rehman, S., Nawaz, Z. & Riazuddin, S. Lysosomotropic agents as HCV entry inhibitors. Virology Journal 8, 163 (2011).
26. Lernia, G. di et al. Clinical Medicine Bortezomib Treatment Modulates Autophagy in Multiple Myeloma. doi: 10.3390/jcm9020552.
27. Takenokuchi, M., Miyamoto, K., Saigo, K. & Taniguchi, T. Bortezomib Causes ER Stress-related Death of Acute Promyelocytic Leukemia Cells Through Excessive Accumulation of PML-RARA. Anticancer Res 35, 3307-16 (2015).
28. Abu-Remaileh, M. et al. Lysosomal metabolomics reveals V-ATPase-and mTOR-dependent regulation of amino acid efflux from lysosomes. Science (1979) 358, 807-813 (2017).
29. Zhang, Q., Jeppesen, D. K. & Higginbotham, J. N. [symbol] . Supermeres are functional extracellular nanoparticles replete with disease biomarkers and therapeutic targets. doi:10.1038/s41556-021-00805-8.
30. Thompson, D. B., Cronican, J. J. & Liu, D. R. Engineering and identifying supercharged proteins for macromolecule delivery into mammalian cells. in Methods in Enzymology 503, 293-319 (Academic Press Inc., 2012).
31. Madisen, L. et al. A robust and high-throughput Cre reporting and characterization system for the whole mouse brain. Nature Neuroscience 13, 133-140 (2010).
32. Said Hassane, F., Saleh, A. F., Abes, R., Gait, M. J. & Lebleu, B. Cell penetrating peptides: Overview and applications to the delivery of oligonucleotides. Cellular and Molecular Life Sciences 67, 715-726 (2010).
33. Kim, Y. B., Zhao, K. T., Thompson, D. B. & Liu, D. R. An anionic human protein mediates cationic liposome delivery of genome editing proteins into mammalian cells. Nature Communications 10, 1-8 (2019).
34. Sharma, R., Huang, X., Brekken, R. A. & Schroit, A. J. Detection of phosphatidylserine-positive exosomes for the diagnosis of early-stage malignancies. British Journal of Cancer 117, 545-552 (2017).
35. Zhang, H. et al. Identification of distinct nanoparticles and subsets of extracellular vesicles by asymmetric flow field-flow fractionation. Nature Cell Biology 20, 332-343 (2018).
36. Fuchs, S. M. & Raines, R. T. Pathway for Polyarginine Entry into Mammalian Cells. Biochemistry 43, 2438-2444 (2004).
37. Williams, C. et al. Assessing the role of surface glycans of extracellular vesicles on cellular uptake. Scientific Reports 9, 1-14 (2019).
38. Huotari, J. & Helenius, A. Endosome maturation. EMBO Journal 30, 3481-3500 (2011).
39. Kudo, K. et al. Cell surface CD63 increased by up-regulated polylactosamine modification sensitizes human melanoma cells to the BRAF inhibitor PLX4032. FASEB Journal 33, 3851-3869 (2019).
40. van den Boorn, J. G., Schlee, M., Coch, C. & Hartmann, G. SiRNA delivery with exosome nanoparticles. Nature Biotechnology 29, 325-326 (2011).
41. Alvarez-Erviti, L. et al. Delivery of siRNA to the mouse brain by systemic injection of targeted exosomes. Nature Biotechnology (2011) doi: 10.1038/nbt. 1807.
42. Tünnemann, G. et al. Live-cell analysis of cell penetration ability and toxicity of oligo-arginines. Journal of Peptide Science 14, 469-476 (2008).
43. Bartnicki, F., Bonarek, P., Kowalska, E. & Strzalka, W. The Argi system: One-step purification of proteins tagged with arginine-rich cell-penetrating peptides. Scientific Reports 7, 1-12 (2017).
44. Brock, D. J. et al. Efficient cell delivery mediated by lipid-specific endosomal escape of supercharged branched peptides. Traffic 19, 421-435 (2018).
45. Cronican, J. J. et al. Potent delivery of functional proteins into mammalian cells in vitro and in vivo using a supercharged protein. ACS Chemical Biology 5, 747-752 (2010).
46. Stearns, N. A., Zhou, S., Petri, M., Binder, S. R. & Pisetsky, D. S. The Use of Poly-L-Lysine as a Capture Agent to Enhance the Detection of Antinuclear Antibodies by ELISA. PLOS ONE 11, e0161818 (2016).
47. Kim, S. M. et al. Cancer-derived exosomes as a delivery platform of CRISPR/Cas9 confer cancer cell tropism-dependent targeting. Journal of Controlled Release 266, 8-16 (2017).
48. Sutaria, D. S. et al. Low active loading of cargo into engineered extracellular vesicles results in inefficient miRNA mimic delivery. Journal of Extracellular Vesicles 6, 1333882 (2017).
49. Deshantri, A. K. et al. Liposomal dexamethasone inhibits tumor growth in an advanced human-mouse hybrid model of multiple myeloma. Journal of Controlled Release 296, 232-240 (2019).
50. Rufino-Ramos, D. et al. Using genetically modified extracellular vesicles as a non-invasive strategy to evaluate brain-specific cargo. Biomaterials 281, 121366 (2022).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising biovesicles, wherein the biovesicles comprise a lipid bilayer surrounding an aqueous lumen; a positively supercharged protein; and one or more cargo molecules complexed with the positively supercharged protein.

2. The composition of claim 1, wherein the biovesicles are extracellular vesicles (EVs), lentiviral vectors (LVVs), or virus-like particles (VLPs).

3. The composition of claim 2, wherein the EVs are obtained from a biofluid or tissue obtained from a living animal, preferably a mammal, more preferably a human.

4. The composition of claim 2, wherein the EVs are obtained from media in a cell culture.

5. The composition of claim 1, wherein the positively supercharged protein comprises supercharged eGFP or a variant thereof, preferably +scmCerulean3 or +scGFP.

6. The composition of claim 5, wherein the +scmCerulean3 is at least 95% identical to SEQ ID NO:4, and has a predicted surface charge of at least +11.

7. The composition of claim 5, wherein the +scGFP is at least 95% identical to SEQ ID NO:4, and has a predicted surface charge of at least +11.

8. The composition of claim 1, wherein the cargo comprises a nucleic acid, a protein, a Ribonucleoprotein (RNP), a combination of DNA and protein, or a small molecules.

9. The composition of claim 8, wherein the nucleic acid is DNA or RNA.

10. The composition of claim 8, wherein the cargo is a protein.

11. The composition of claim 10, wherein the protein is a genome editing or epigenome modulating protein.

12. The composition of claim 11, wherein the genome editing or epigenome modulating protein is or comprises a CRISPR based nuclease, zinc finger (ZF) or a TALE, optionally fused to an epigenome modulator.

13. The composition of claim 10, wherein the positively supercharged protein and the protein cargo are in a fusion protein, optionally wherein the positively supercharged protein is fused to the N or C terminus of the cargo, optionally with a polypeptide linker therebetween.

14. The composition of claim 10, wherein the protein is or comprises a CRISPR based nuclease, and the cargo further comprises a guide RNA complexed with the CRISPR based nuclease.

15. A method of delivering a cargo to a cell or tissues, the method comprising contacting the cell or tissue with the composition of claim 1.

16. A reporter construct comprising:
a transmembrane domain protein;
a reporter gene fused to an intracellular portion of the transmembrane domain protein;
an affinity tag fused to an extracellular portion of the transmembrane domain protein;
and a nucleotide sequence complementary to a target gRNA sequence comprising a premature stop codon, wherein the premature stop codon prevents expression of the reporter construct.

17. The reporter construct of claim 16, wherein the transmembrane domain protein comprises at least two transmembrane domains and at least one extracellular loop, and wherein the affinity tag is disposed in the at least one extracellular loop.

18. The reporter construct of claim 16, wherein the transmembrane domain is CD63 tetraspanin.

19. The reporter construct of claim 16, wherein the reporter gene comprises one or both of a fluorescent tag and a bioluminescent tag.

20. A method of detecting CRISPR editing of a sequence comprising a premature stop codon in a genome of a cell, the method comprising:
expressing in the cell with the reporter construct of claim 16;
isolating extracellular vesicles EVs from the cell; and
detecting expression of the reporter construct by detecting a signal from the reporter gene.

21. The method of claim 20, further comprising isolating the EVs or cells expressing the construct by contacting the cells or EVs with an affinity-tag binding reagent, and isolating cells or EVs that comprise the affinity tag.


SEQUENCE LISTING (10 sequences)

SEQ ID NO: 1 - protein, 239 aa, Artificial Sequence
  eGFP
MVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 2 - protein, 239 aa, Artificial Sequence
  positive supercharged eGFP
MASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYK

SEQ ID NO: 3 - protein, 238 aa, Artificial Sequence
  negative supercharged eGFP
MSKGEELFDGVVPILVELDGDVNGHEFSVRGEGEGDATEGELTLKFICTTGELPVPWPTLVTTLTYGVQCFSDYPDHMDQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEDRDHMVLLEFVTAAGIDHGMDELYK

SEQ ID NO: 4 - protein, 239 aa, Artificial Sequence
  supercharged mCerulean3
MVSKGERLFRGKVPILVELKGDVNGHKFSVRGKGKGDATRGKLTLKFICTTGKLPVPWPTLVTTLSWGVQCFARYPKHMKRHDFFKSAMPKGYVQERTISFKKDGKYKTRAEVKFEGRTLVNRIKLKGRDFKEKGNILGHKLRYNAIHGKVYITADKRKNGIKAKFGLNCNVKDGSVQLADHYQQNTPIGRGPVLLPRNHYLSTRSKLSKDPKEKRDHMVLLEFVTAAGIKLGRDERYK

SEQ ID NO: 5 - protein, 239 aa, Aequorea victoria
  MISC_FEATURE (1)..(239): wild type mCerulean3
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNAIHGNVYITADKQKNGIKANFGLNCNIEDGSVQLADHYQQNTPIGDGPVLLPDNHYLSTQSKLSKDPNEKRDHMVLLEFVTAAGITLGMDELYK

SEQ ID NO: 6 - protein, 7 aa, Artificial Sequence
  SV40 NLS
PKKKRKV

SEQ ID NO: 7 - protein, 16 aa, Artificial Sequence
  nucleoplasmin NLS
KRPAATKKAGQAKKKK

SEQ ID NO: 8 - protein, 8 aa, Artificial Sequence
  Flag-Tag
DYDKDDDK

SEQ ID NO: 9 - protein, 9987 aa, Artificial Sequence
  NoMi sequence
ACGCGTGTAGTCTTATGCAATACTCTTGTAGTCTTGCAACATGGTAACGATGAGTTAGCAACATGCCTTACAAGGAGAGAAAAAGCACCGTGCATGCCGATTGGTGGAAGTAAGGTGGTACGATCGTGCCTTATTAGGAAGGCAACAGACGGGTCTGACATGGATTGGACGAACCACTGAATTGCCGCATTGCAGAGATATTGTATTTAAGTGCCTAGCTCGATACAATAAACGGGTCTCTCTGGTTAGACCAGATCTGAGCCTGGGAGCTCTCTGGCTAACTAGGGAACCCACTGCTTAAGCCTCAATAAAGCTTGCCTTGAGTGCTTCAAGTAGTGTGTGCCCGTCTGTTGTGTGACTCTGGTAACTAGAGATCCCTCAGACCCTTTTAGTCAGTGTGGAAAATCTCTAGCAGTGGCGCCCGAACAGGGACTTGAAAGCGAAAGGGAAACCAGAGGAGCTCTCTCGACGCAGGACTCGGCTTGCTGAAGCGCGCACGGCAAGAGGCGAGGGGCGGCGACTGGTGAGTACGCCAAAAATTTTGACTAGCGGAGGCTAGAAGGAGAGAGATGGGTGCGAGAGCGTCAGTATTAAGCGGGGGAGAATTAGATCGCGATGGGAAAAAATTCGGTTAAGGCCAGGGGGAAAGAAAAAATATAAATTAAAACATATAGTATGGGCAAGCAGGGAGCTAGAACGATTCGCAGTTAATCCTGGCCTGTTAGAAACATCAGAAGGCTGTAGACAAATACTGGGACAGCTACAACCATCCCTTCAGACAGGATCAGAAGAACTTAGATCATTATATAATACAGTAGCAACCCTCTATTGTGTGCATCAAAGGATAGAGATAAAAGACACCAAGGAAGCTTTAGACAAGATAGAGGAAGAGCAAAACAAAAGTAAGACCACCGCACAGCAAGCGGCCACTGATCTTCAGACCTGGAGGAGGAGATATGAGGGACAATTGGAGAAGTGAATTATATAAATATAAAGTAGTAAAAATTGAACCATTAGGAGTAGCACCCACCAAGGCAAAGAGAAGAGTGGTGCAGAGAGAAAAAAGAGCAGTGGGAATAGGAGCTTTGTTCCTTGGGTTCTTGGGAGCAGCAGGAAGCACTATGGGCGCAGCGTCAATGACGCTGACGGTACAGGCCAGACAATTATTGTCTGGTATAGTGCAGCAGCAGAACAATTTGCTGAGGGCTATTGAGGCGCAACAGCATCTGTTGCAACTCACAGTCTGGGGCATCAAGCAGCTCCAGGCAAGAATCCTGGCTGTGGAAAGATACCTAAAGGATCAACAGCTCCTGGGGATTTGGGGTTGCTCTGGAAAACTCATTTGCACCACTGCTGTGCCTTGGAATGCTAGTTGGAGTAATAAATCTCTGGAACAGATTTGGAATCACACGACCTGGATGGAGTGGGACAGAGAAATTAACAATTACACAAGCTTAATACACTCCTTAATTGAAGAATCGCAAAACCAGCAAGAAAAGAATGAACAAGAATTATTGGAATTAGATAAATGGGCAAGTTTGTGGAATTGGTTTAACATAACAAATTGGCTGTGGTATATAAAATTATTCATAATGATAGTAGGAGGCTTGGTAGGTTTAAGAATAGTTTTTGCTGTACTTTCTATAGTGAATAGAGTTAGGCAGGGATATTCACCATTATCGTTTCAGACCCACCTCCCAACCCCGAGGGGACCCGACAGGCCCGAAGGAATAGAAGAAGAAGGTGGAGAGAGAGACAGAGACAGATCCATTCGATTAGTGAACGGATCTCGACGGTATCGGTTAACTTTTAAAAGAAAAGGGGGGATTGGGGGGTACAGTGCAGGGGAAAGAATAGTAGACATAATAGCAACAGACATACAAACTAAAGAATTACAAAAACAAATTACAAAATTCAAAATTTTATCGATACTAGTATTATGCCCAGTACATGACCTTATGGGACTTTCCTACTTGGCAGTACATCTACGTATTAGTCATCGCTATTACCATGGTGATGCGGTTTTGGCAGTACATCAATGGGCGTGGATAGCGGTTTGACTCACGGGGATTTCCAAGTCTCCACCCCATTGACGTCAATGGGAGTTTGTTTTGGCACCAAAATCAACGGGACTTTCCAAAATGTCGTAACAACTCCGCCCCATTGACGCAAATGGGCGGTAGGCGTGTACGGTGGGAGGTTTATATAAGCAGAGCTCGTTTAGTGAACCGTCAGATCGCCTGGAGACGCCATCCACGCTGTTTTGACCTCCATAGAAGATTGCGGCCGCATGGCGGTGGAAGGAGGAATGAAATGTGTGAAGTTCTTGCTCTACGTCCTCCTGCTGGCCTTTTGCGCCTGTGCAGTGGGACTGATTGCCGTGGGTGTCGGGGCACAGCTTGTCCTGAGTCAGACCATATCTAGAGACTACAAAGACCATGACGGTGATTATAAAGATCATGACATCGATTACAAGGATGACGATGACAAGGGTGGTTCTGGTGGTGGTTCTGGTCGATCCACCATGGTCTTCACACTCGAAGATTTCGTTGGGGACTGGCGACAGACAGCCGGCTACAACCTGGACCAAGTCCTTGAACAGGGAGGTGTGTCCAGTTTGTTTCAGAATCTCGGGGTGTCCGTAACTCCGATCCAAAGGATTGTCCTGAGCGGTGAAAATGGGCTGAAGATCGACATCCATGTCATCATCCCGTATGAAGGTCTGAGCGGCGACCAAATGGGCCAGATCGAAAAAATTTTTAAGGTGGTGTACCCTGTGGATGATCATCACTTTAAGGTGATCCTGCACTATGGCACACTGGTAATCGACGGGGTTACGCCGAACATGATCGACTATTTCGGACGGCCGTATGAAGGCATCGCCGTGTTCGACGGCAAAAAGATCACTGTAACAGGGACCCTGTGGAACGGCAACAAAATTATCGACGAGCGCCTGATCAACCCCGACGGCTCCCTGCTGTTCCGAGTAACCATCAACGGAGTGACCGGCTGGCGGCTGTGCGAACGCATTCTGGCGTCTAGAATCCAGGGGGCTACCCCTGGCTCTCTGTTGCCAGTGGTCATCATCGCAGTGGGTGTCTTCCTCTTCCTGGTGGCTTTTGTGGGCTGCTGCGGGGCCTGCAAGGAGAACTATTGTCTTATGATCACGTTTGCCATCTTTCTGTCTCTTATCATGTTGGTGGAGGTGGCCGCAGCCATTGCTGGCTATGTGTTTAGAGATAAGGTGATGTCAGAGTTTAATAACAACTTCCGGCAGCAGATGGAGAATTACCCGAAAAATAACCACACTGCTTCGATCCTGGACAGGATGCAGGCAGATTTTAAGTGCTGTGGGGCTGCTAACTACACAGATTGGGAGAAAATCCCTTCCATGTCGAAGAACCGAGTCCCCGACTCCTGCTGCATTAATGTTACTGTGGGCTGTGGGATTAATTTCAACGAGAAGGCGATCCATAAGGAGGGCTGTGTGGAGAAGATTGGGGGCTGGCTGAGGAAAAATGTGCTGGTGGTAGCTGCAGCAGCCCTTGGAATTGCTTTTGTCGAGGTTTTGGGAATTGTCTTTGCCTGCTGCCTTGTGAAGAGTATCAGAAGTGGCTACGAGGTGATGGAATTCGCTAGCGCTACCGGACTCAGATCTCGAGCTCAAGCTTCGAATTCTGCAGTCGACGGTACCGCGGGCCCGGGATCCACCGGTATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGGCTAGCGCTACCGGACTCAGATCTCGAGCTCAAGCTTCGAATTCTGCAGTCGACGGTACCGCGGGCCCGGGATCCACCGGTAAGCTTGGAAGCGGAGCTACTAACTTCAGCCTGCTGAAGCAGGCTGGAGACGTGGAGGAGAACCCTGGACCTATGGAGAGCGACGAGAGCGGCCTGCCCGCCATGGAGATCGAGTGCCGCATCACCGGCACCCTGAACGGCGTGGAGTTCGAGCTGGTGGGCGGCGGAGAGGGCACCCCCAAGCAGGGCCGCATGACCAACAAGATGAAGAGCACCAAAGGCGCCCTGACCTTCAGCCCCTACCTGCTGAGCCACGTGATGGGCTACGGCTTCTACCACTTCGGCACCTACCCCAGCGGCTACGAGAACCCCTTCCTGCACGCCATCAACAACGGCGGCTACACCAACACCCGCATCGAGAAGTACGAGGACGGCGGCGTGCTGCACGTGAGCTTCAGCTACCGCTACGAGGCCGGCCGCGTGATCGGCGACTTCAAGGTGGTGGGCACCGGCTTCCCCGAGGACAGCGTGATCTTCACCGACAAGATCATCCGCAGCAACGCCACCGTGGAGCACCTGCACCCCATGGGCGATAACGTGCTGGTGGGCAGCTTCGCCCGCACCTTCAGCCTGCGCGACGGCGGCTACTACAGCTTCGTGGTGGACAGCCACATGCACTTCAAGAGCGCCATCCACCCCAGCATCCTGCAGAACGGGGGCCCCATGTTCGCCTTCCGCCGCGTGGAGGAGCTGCACAGCAACACCGAGCTGGGCATCGTGGAGTACCAGCACGCCTTCAAGACCCCCATTGCCTTCGCCAGATCCCGCGCTCAGTCGTCCAATTCTGCCGTGGACGGCACCGCCGGACCCGGCTCCACCGGATCTCGCGTCGACGGCAGTGGAGAGGGCAGAGGAAGTCTGCTAACATGCGGTGACGTCGAGGAGAATCCTGGCCCAATGACCGAGTACAAGCCCACGGTGCGCCTCGCCACCCGCGACGACGTCCCCAGGGCCGTACGCACCCTCGCCGCCGCGTTCGCCGACTACCCCGCCACGCGCCACACCGTCGATCCGGACCGCCACATCGAGCGGGTCACCGAGCTGCAAGAACTCTTCCTCACGCGCGTCGGGCTCGACATCGGCAAGGTGTGGGTCGCGGACGACGGCGCCGCGGTGGCGGTCTGGACCACGCCGGAGAGCGTCGAAGCGGGGGCGGTGTTCGCCGAGATCGGCCCGCGCATGGCCGAGTTGAGCGGTTCCCGGCTGGCCGCGCAGCAACAGATGGAAGGCCTCCTGGCGCCGCACCGGCCCAAGGAGCCCGCGTGGTTCCTGGCCACCGTCGGCGTCTCGCCCGACCACCAGGGCAAGGGTCTGGGCAGCGCCGTCGTGCTCCCCGGAGTGGAGGCGGCCGAGCGCGCCGGGGTGCCCGCCTTCCTGGAGACCTCCGCGCCCCGCAACCTCCCCTTCTACGAGCGGCTCGGCTTCACCGTCACCGCCGACGTCGAGGTGCCCGAAGGACCGCGCACCTGGTGCATGACCCGCAAGCCCGGTGCCTGAGAATTCGTCGACAATCAACCTCTGGATTACAAAATTTGTGAAAGATTGACTGGTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTATAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGGGCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGGGGCTCGGCTGTTGGGCACTGACAATTCCGTGGTGTTGTCGGGGAAATCATCGTCCTTTCCTTGGCTGCTCGCCTGTGTTGCCACCTGGATTCTGCGCGGGACGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAGACGAGTCGGATCTCCCTTTGGGCCGCCTCCCCGCCTGGTACCTTTAAGACCAATGACTTACAAGGCAGCTGTAGATCTTAGCCACTTTTTAAAAGAAAAGGGGGGACTGGAAGGGCTAATTCACTCCCAACGAAGATAAGATCTGCTTTTTGCTTGTACTGGGTCTCTCTGGTTAGACCAGATCTGAGCCTGGGAGCTCTCTGGCTAACTAGGGAACCCACTGCTTAAGCCTCAATAAAGCTTGCCTTGAGTGCTTCAAGTAGTGTGTGCCCGTCTGTTGTGTGACTCTGGTAACTAGAGATCCCTCAGACCCTTTTAGTCAGTGTGGAAAATCTCTAGCAGTAGTAGTTCATGTCATCTTATTATTCAGTATTTATAACTTGCAAAGAAATGAATATCAGAGAGTGAGAGGAACTTGTTTATTGCAGCTTATAATGGTTACAAATAAAGCAATAGCATCACAAATTTCACAAATAAAGCATTTTTTTCACTGCATTCTAGTTGTGGTTTGTCCAAACTCATCAATGTATCTTATCATGTCTGGCTCTAGCTATCCCGCCCCTAACTCCGCCCATCCCGCCCCTAACTCCGCCCAGTTCCGCCCATTCTCCGCCCCATGGCTGACTAATTTTTTTTATTTATGCAGAGGCCGAGGCCGCCTCGGCCTCTGAGCTATTCCAGAAGTAGTGAGGAGGCTTTTTTGGAGGCCTAGACTTTTGCAGAGACCAAATTCGTAATCATGTCATAGCTGTTTCCTGTGTGAAATTGTTATCCGCTCACAATTCCACACAACATACGAGCCGGAAGCATAAAGTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGGCGCTCTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAATCTAAAGTATATATGAGTAAACTTGGTCTGACAGTTACCAATGCTTAATCAGTGAGGCACCTATCTCAGCGATCTGTCTATTTCGTTCATCCATAGTTGCCTGACTCCCCGTCGTGTAGATAACTACGATACGGGAGGGCTTACCATCTGGCCCCAGTGCTGCAATGATACCGCGAGACCCACGCTCACCGGCTCCAGATTTATCAGCAATAAACCAGCCAGCCGGAAGGGCCGAGCGCAGAAGTGGTCCTGCAACTTTATCCGCCTCCATCCAGTCTATTAATTGTTGCCGGGAAGCTAGAGTAAGTAGTTCGCCAGTTAATAGTTTGCGCAACGTTGTTGCCATTGCTACAGGCATCGTGGTGTCACGCTCGTCGTTTGGTATGGCTTCATTCAGCTCCGGTTCCCAACGATCAAGGCGAGTTACATGATCCCCCATGTTGTGCAAAAAAGCGGTTAGCTCCTTCGGTCCTCCGATCGTTGTCAGAAGTAAGTTGGCCGCAGTGTTATCACTCATGGTTATGGCAGCACTGCATAATTCTCTTACTGTCATGCCATCCGTAAGATGCTTTTCTGTGACTGGTGAGTACTCAACCAAGTCATTCTGAGAATAGTGTATGCGGCGACCGAGTTGCTCTTGCCCGGCGTCAATACGGGATAATACCGCGCCACATAGCAGAACTTTAAAAGTGCTCATCATTGGAAAACGTTCTTCGGGGCGAAAACTCTCAAGGATCTTACCGCTGTTGAGATCCAGTTCGATGTAACCCACTCGTGCACCCAACTGATCTTCAGCATCTTTTACTTTCACCAGCGTTTCTGGGTGAGCAAAAACAGGAAGGCAAAATGCCGCAAAAAAGGGAATAAGGGCGACACGGAAATGTTGAATACTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCCCGAAAAGTGCCACCTGACGTCTAAGAAACCATTATTATCATGACATTAACCTATAAAAATAGGCGTATCACGAGGCCCTTTCGTCTCGCGCGTTTCGGTGATGACGGTGAAAACCTCTGACACATGCAGCTCCCGGAGACGGTCACAGCTTGTCTGTAAGCGGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGCGGTGTGAAATACCGCACAGATGCGTAAGGAGAAAATACCGCATCAGGCGCCATTCGCCATTCAGGCTGCGCAACTGTTGGGAAGGGCGATCGGTGCGGGCCTCTTCGCTATTACGCCAGCTGGCGAAAGGGGGATGTGCTGCAAGGCGATTAAGTTGGGTAACGCCAGGGTTTTCCCAGTCACGACGTTGTAAAACGACGGCCAGTGCCAAGCTGACGCGTGTAGTCTTATGCAATACTCTTGTAGTCTTGCAACATGGTAACGATG

SEQ ID NO: 10 - protein, 11566 aa, Artificial Sequence
  CRISPR reporter lentiviral vector
CCAAGTCATTCTGAGAATAGTGTATGCGGCGACCGAGTTGCTCTTGCCCGGCGTCAATACGGGATAATACCGCGCCACATAGCAGAACTTTAAAAGTGCTCATCATTGGAAAACGTTCTTCGGGGCGAAAACTCTCAAGGATCTTACCGCTGTTGAGATCCAGTTCGATGTAACCCACTCGTGCACCCAACTGATCTTCAGCATCTTTTACTTTCACCAGCGTTTCTGGGTGAGCAAAAACAGGAAGGCAAAATGCCGCAAAAAAGGGAATAAGGGCGACACGGAAATGTTGAATACTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCCCGAAAAGTGCCACCTGACGTCGACGGATCGGGAGATCTCCCGATCCCCTATGGTGCACTCTCAGTACAATCTGCTCTGATGCCGCATAGTTAAGCCAGTATCTGCTCCCTGCTTGTGTGTTGGAGGTCGCTGAGTAGTGCGCGAGCAAAATTTAAGCTACAACAAGGCAAGGCTTGACCGACAATTGCATGAAGAATCTGCTTAGGGTTAGGCGTTTTGCGCTGCTTCGCGATGTACGGGCCAGATATACGCGTTGACATTGATTATTGACTAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGTACATGACCTTATGGGACTTTCCTACTTGGCAGTACATCTACGTATTAGTCATCGCTATTACCATGGTGATGCGGTTTTGGCAGTACATCAATGGGCGTGGATAGCGGTTTGACTCACGGGGATTTCCAAGTCTCCACCCCATTGACGTCAATGGGAGTTTGTTTTGGCACCAAAATCAACGGGACTTTCCAAAATGTCGTAACAACTCCGCCCCATTGACGCAAATGGGCGGTAGGCGTGTACGGTGGGAGGTCTATATAAGCAGCGCGTTTTGCCTGTACTGGGTCTCTCTGGTTAGACCAGATCTGAGCCTGGGAGCTCTCTGGCTAACTAGGGAACCCACTGCTTAAGCCTCAATAAAGCTTGCCTTGAGTGCTTCAAGTAGTGTGTGCCCGTCTGTTGTGTGACTCTGGTAACTAGAGATCCCTCAGACCCTTTTAGTCAGTGTGGAAAATCTCTAGCAGTGGCGCCCGAACAGGGACTTGAAAGCGAAAGGGAAACCAGAGGAGCTCTCTCGACGCAGGACTCGGCTTGCTGAAGCGCGCACGGCAAGAGGCGAGGGGCGGCGACTGGTGAGTACGCCAAAAATTTTGACTAGCGGAGGCTAGAAGGAGAGAGATGGGTGCGAGAGCGTCAGTATTAAGCGGGGGAGAATTAGATCGCGATGGGAAAAAATTCGGTTAAGGCCAGGGGGAAAGAAAAAATATAAATTAAAACATATAGTATGGGCAAGCAGGGAGCTAGAACGATTCGCAGTTAATCCTGGCCTGTTAGAAACATCAGAAGGCTGTAGACAAATACTGGGACAGCTACAACCATCCCTTCAGACAGGATCAGAAGAACTTAGATCATTATATAATACAGTAGCAACCCTCTATTGTGTGCATCAAAGGATAGAGATAAAAGACACCAAGGAAGCTTTAGACAAGATAGAGGAAGAGCAAAACAAAAGTAAGACCACCGCACAGCAAGCGGCCGGCCGCTGATCTTCAGACCTGGAGGAGGAGATATGAGGGACAATTGGAGAAGTGAATTATATAAATATAAAGTAGTAAAAATTGAACCATTAGGAGTAGCACCCACCAAGGCAAAGAGAAGAGTGGTGCAGAGAGAAAAAAGAGCAGTGGGAATAGGAGCTTTGTTCCTTGGGTTCTTGGGAGCAGCAGGAAGCACTATGGGCGCAGCGTCAATGACGCTGACGGTACAGGCCAGACAATTATTGTCTGGTATAGTGCAGCAGCAGAACAATTTGCTGAGGGCTATTGAGGCGCAACAGCATCTGTTGCAACTCACAGTCTGGGGCATCAAGCAGCTCCAGGCAAGAATCCTGGCTGTGGAAAGATACCTAAAGGATCAACAGCTCCTGGGGATTTGGGGTTGCTCTGGAAAACTCATTTGCACCACTGCTGTGCCTTGGAATGCTAGTTGGAGTAATAAATCTCTGGAACAGATTTGGAATCACACGACCTGGATGGAGTGGGACAGAGAAATTAACAATTACACAAGCTTAATACACTCCTTAATTGAAGAATCGCAAAACCAGCAAGAAAAGAATGAACAAGAATTATTGGAATTAGATAAATGGGCAAGTTTGTGGAATTGGTTTAACATAACAAATTGGCTGTGGTATATAAAATTATTCATAATGATAGTAGGAGGCTTGGTAGGTTTAAGAATAGTTTTTGCTGTACTTTCTATAGTGAATAGAGTTAGGCAGGGATATTCACCATTATCGTTTCAGACCCACCTCCCAACCCCGAGGGGACCCGACAGGCCCGAAGGAATAGAAGAAGAAGGTGGAGAGAGAGACAGAGACAGATCCATTCGATTAGTGAACGGATCGGCACTGCGTGCGCCAATTCTGCAGACAAATGGCAGTATTCATCCACAATTTTAAAAGAAAAGGGGGGATTGGGGGGTACAGTGCAGGGGAAAGAATAGTAGACATAATAGCAACAGACATACAAACTAAAGAATTACAAAAACAAATTACAAAAATTCAAAATTTTCGGGTTTATTACAGGGACAGCAGAGATCCAGTTTGGTTAGTACCGGGCCCGCTCTAGCGTGAGGCTCCGGTGCCCGTCAGTGGGCAGAGCGCACATCGCCCACAGTCCCCGAGAAGTTGGGGGGAGGGGTCGGCAATTGAACCGGTGCCTAGAGAAGGTGGCGCGGGGTAAACTGGGAAAGTGATGTCGTGTACTGGCTCCGCCTTTTTCCCGAGGGTGGGGGAGAACCGTATATAAGTGCAGTAGTCGCCGTGAACGTTCTTTTTCGCAACGGGTTTGCCGCCAGAACACAGGTAAGTGCCGTGTGTGGTTCCCGCGGGCCTGGCCTCTTTACGGGTTATGGCCCTTGCGTGCCTTGAATTACTTCCACCTGGCTGCAGTACGTGATTCTTGATCCCGAGCTTCGGGTTGGAAGTGGGTGGGAGAGTTCGAGGCCTTGCGCTTAAGGAGCCCCTTCGCCTCGTGCTTGAGTTGAGGCCTGGCCTGGGCGCTGGGGCCGCCGCGTGCGAATCTGGTGGCACCTTCGCGCCTGTCTCGCTGCTTTCGATAAGTCTCTAGCCATTTAAAATTTTTGATGACCTGCTGCGACGCTTTTTTTCTGGCAAGATAGTCTTGTAAATGCGGGCCAAGATCTGCACACTGGTATTTCGGTTTTTGGGGCCGCGGGCGGCGACGGGGCCCGTGCGTCCCAGCGCACATGTTCGGCGAGGCGGGGCCTGCGAGCGCGGCCACCGAGAATCGGACGGGGGTAGTCTCAAGCTGGCCGGCCTGCTCTGGTGCCTGGCCTCGCGCCGCCGTGTATCGCCCCGCCCTGGGCGGCAAGGCTGGCCCGGTCGGCACCAGTTGCGTGAGCGGAAAGATGGCCGCTTCCCGGCCCTGCTGCAGGGAGCTCAAAATGGAGGACGCGGCGCTCGGGAGAGCGGGCGGGTGAGTCACCCACACAAAGGAAAAGGGCCTTTCCGTCCTCAGCCGTCGCTTCATGTGACTCCACGGAGTACCGGGCGCCGTCCAGGCACCTCGATTAGTTCTCGAGCTTTTGGAGTACGTCGTCTTTAGGTTGGGGGGAGGGGTTTTATGCGATGGAGTTTCCCCACACTGAGTGGGTGGAGACTGAAGTTAGGCCAGCTTGGCACTTGATGTAATTCTCCTTGGAATTTGCCCTTTTTGAGTTTGGATCTTGGTTCATTCTCAAGCCTCAGACAGTGGTTCAAAGTTTTTTTCTTCCATTTCAGGTGTCGTGAGCGGCCGCTGAGTTAACTATTATGGCGGTGGAAGGAGGAATGAAATGTGTGAAGTTCTTGCTCTACGTCCTCCTGCTGGCCTTTTGCGCCTGTGCAGTGGGACTGATTGCCGTGGGTGTCGGGGCACAGCTTGTCCTGAGTCAGACCTAAAAGGTCATACCCATACGATGTTCCTGACTATGCGGGCTATCCCTATGACGTCCCGGACTATGCAGGATCCTATCCATATGACGTTCCAGATTACGCTGTTAACTCTGGTGTAGCAGGTCATGCCTCTGGCAGCCCCGCATTCGGGACCGCCTCTCATTCGAATTGCGAACATGAAGAGATCCACCTCGCCGGCTCGATCCAGCCGCATGGCGCGCTTCTGGTCGTCAGCGAACATGATCATCGCGTCATCCAGGCCAGCGCCAACGCCGCGGAATTTCTGAATCTCGGAAGCGTACTCGGCGTTCCGCTCGCCGAGATCGACGGCGATCTGTTGATCAAGATCCTGCCGCATTTAGATCCAACCGCAGAAGGCATGCCGGTCGCGGTGCGCTGCCGGATCGGTAATCCCTCTACGGAGTACTGCGGTCTGATGCATCGGCCTCCGGAAGGCGGTCTGATCATCGAACTCGAACGTGCAGGACCATCGATCGATCTGTCAGGCACGCTGGCGCCGGCGTTAGAGCGGATCCGCACGGCGGGTTCACTGCGCGCACTGTGTGATGACACAGTACTGCTGTTTCAGCAGTGCACCGGCTACGACCGGGTGATGGTGTATCGTTTCGATGAGCAAGGCCACGGATTAGTATTCTCCGAGTGCCATGTGCCTGGGCTCGAATCCTATTTCGGCAACCGCTATCCGTCGTCGCTGGTCCCGCAGATGGCAAGACAGCTGTACGTGAGACAGCGCGTCCGCGTGCTGGTCGACGTAACTTATCAACCAGTGCCGCTGGAGCCACGGCTGTCGCCGCTGACTGGAAGAGATCTCGACATGTCGGGCTGCTTCCTGCGCTCGATGTCGCCGATCCATTTACAGTTCCTGAAGGACATGGGCGTGCGCGCCACCCTAGCAGTTTCACTGGTGGTCGGCGGCAAGCTGTGGGGCCTGGTTGTCTGTCACCATTATCTGCCGCGCTTCATCCGTTTCGAGTTACGGGCGATCTGCAAACGGCTCGCCGAAAGGATCGCGACGAGAATAACCGCACTTGAATCCATCCAGGGGGCTACCCCTGGCTCTCTGTTGCCAGTGGTCATCATCGCAGTGGGTGTCTTCCTCTTCCTGGTGGCTTTTGTGGGCTGCTGCGGGGCCTGCAAGGAGAACTATTGTCTTATGATCACGTTTGCCATCTTTCTGTCTCTTATCATGTTGGTGGAGGTGGCCGCAGCCATTGCTGGCTATGTGTTTAGAGATAAGGTGATGTCAGAGTTTAATAACAACTTCCGGCAGCAGATGGAGAATTACCCGAAAAATAACCACACTGCTTCGATCCTGGACAGGATGCAGGCAGATTTTAAGTGCTGTGGGGCTGCTAACTACACAGATTGGGAGAAAATCCCTTCCATGTCGAAGAACCGAGTCCCCGACTCCTGCTGCATTAATGTTACTGTGGGCTGTGGGATTAATTTCAACGAGAAGGCGATCCATAAGGAGGGCTGTGTGGAGAAGATTGGGGGCTGGCTGAGGAAAAATGTGCTGGTGGTAGCTGCAGCAGCCCTTGGAATTGCTTTTGTCGAGGTTTTGGGAATTGTCTTTGCCTGCTGCCTCGTGAAGAGTATCAGAAGTGGCTACGAGGTGATGGAATTCATGGAAGACGCCAAAAACATAAAGAAAGGCCCGGCGCCATTCTATCCGCTGGAAGATGGAACCGCTGGAGAGCAACTGCATAAGGCTATGAAGAGATACGCCCTGGTTCCTGGAACAATTGCTTTTACAGATGCACATATCGAGGTGGACATCACTTACGCTGAGTACTTCGAAATGTCCGTTCGGTTGGCAGAAGCTATGAAACGATATGGGCTGAATACAAATCACAGAATCGTCGTATGCAGTGAAAACTCTCTTCAATTCTTTATGCCGGTGTTGGGCGCGTTATTTATCGGAGTTGCAGTTGCGCCCGCGAACGACATTTATAATGAACGTGAATTGCTCAACAGTATGGGCATTTCGCAGCCTACCGTGGTGTTCGTTTCCAAAAAGGGGTTGCAAAAAATTTTGAACGTGCAAAAAAAGCTCCCAATCATCCAAAAAATTATTATCATGGATTCTAAAACGGATTACCAGGGATTTCAGTCGATGTACACGTTCGTCACATCTCATCTACCTCCCGGTTTTAATGAATACGATTTTGTGCCAGAGTCCTTCGATAGGGACAAGACAATTGCACTGATCATGAACTCCTCTGGATCTACTGGTCTGCCTAAAGGTGTCGCTCTGCCTCATAGAACTGCCTGCGTGAGATTCTCGCATGCCAGAGATCCTATTTTTGGCAATCAAATCATTCCGGATACTGCGATTTTAAGTGTTGTTCCATTCCATCACGGTTTTGGAATGTTTACTACACTCGGATATTTGATATGTGGATTTCGAGTCGTCTTAATGTATAGATTTGAAGAAGAGCTGTTTCTGAGGAGCCTTCAGGATTACAAGATTCAAAGTGCGCTGCTGGTGCCAACCCTATTCTCCTTCTTCGCCAAAAGCACTCTGATTGACAAATACGATTTATCTAATTTACACGAAATTGCTTCTGGTGGCGCTCCCCTCTCTAAGGAAGTCGGGGAAGCGGTTGCCAAGAGGTTCCATCTGCCAGGTATCAGGCAAGGATATGGGCTCACTGAGACTACATCAGCTATTCTGATTACACCCGAGGGGGATGATAAACCGGGCGCGGTCGGTAAAGTTGTTCCATTTTTTGAAGCGAAGGTTGTGGATCTGGATACCGGGAAAACGCTGGGCGTTAATCAAAGAGGCGAACTGTGTGTGAGAGGTCCTATGATTATGTCCGGTTATGTAAACAATCCGGAAGCGACCAACGCCTTGATTGACAAGGATGGATGGCTACATTCTGGAGACATAGCTTACTGGGACGAAGACGAACACTTCTTCATCGTTGACCGCCTGAAGTCTCTGATTAAGTACAAAGGCTATCAGGTGGCTCCCGCTGAATTGGAATCCATCTTGCTCCAACACCCCAACATCTTCGACGCAGGTGTCGCAGGTCTTCCCGACGATGACGCCGGTGAACTTCCCGCCGCCGTTGTTGTTTTGGAGCACGGAAAGACGATGACGGAAAAAGAGATCGTGGATTACGTCGCCAGTCAAGTAACAACCGCGAAAAAGTTGCGCGGAGGAGTTGTGTTTGTGGACGAAGTACCGAAAGGTCTTACCGGAAAACTCGACGCAAGAAAAATCAGAGAGATCCTCATAAAGGCCAAGAAGGGCGGAAAGATCGCCGTGCTAGAGTACCCGGGCTAGGATCCGCCCCTCTCCCTCCCCCCCCCCTAACGTTACTGGCCGAAGCCGCTTGGAATAAGGCCGGTGTGCGTTTGTCTATATGTTATTTTCCACCATATTGCCGTCTTTTGGCAATGTGAGGGCCCGGAAACCTGGCCCTGTCTTCTTGACGAGCATTCCTAGGGGTCTTTCCCCTCTCGCCAAAGGAATGCAAGGTCTGTTGAATGTCGTGAAGGAAGCAGTTCCTCTGGAAGCTTCTTGAAGACAAACAACGTCTGTAGCGACCCTTTGCAGGCAGCGGAACCCCCCACCTGGCGACAGGTGCCTCTGCGGCCAAAAGCCACGTGTATAAGATACACCTGCAAAGGCGGCACAACCCCAGTGCCACGTTGTGAGTTGGATAGTTGTGGAAAGAGTCAAATGGCTCTCCTCAAGCGTATTCAACAAGGGGCTGAAGGATGCCCAGAAGGTACCCCATTGTATGGGATCTGATCTGGGGCCTCGGTACACATGCTTTACATGTGTTTAGTCGAGGTTAAAAAAACGTCTAGGCCCCCCGAACCACGGGGACGTGGTTTTCCTTTGAAAAACACGATGATAATATGGCCACAACCATGTCACCAGAGCCAGCGAAGTCTGCTCCCGCCCCGAAAAAGGGCTCCAAGAAGGCGGTGACTAAGGCGCAGAAGAAAGGCGGCAAGAAGCGCAAGCGCAGCCGCAAGGAGAGCTATTCCATCTATGTGTACAAGGTTCTGAAGCAGGTCCACCCTGACACCGGCATTTCGTCCAAGGCCATGGGCATCATGAATTCGTTTGTGAACGACATTTTCGAGCGCATCGCAGGTGAGGCTTCCCGCCTGGCGCATTACAACAAGCGCTCGACCATCACCTCCAGGGAGATCCAGACGGCCGTGCGCCTGCTGCTGCCTGGGGAGTTGGCCAAGCACGCCGTGTCCGAGGGTACTAAGGCCATCACCAAGTACACCAGCGCTAAGGATCCACCGGTCGCCACCATGGTGAGCAAGGGCGAGGAGCTGTTCACCGGGGTGGTGCCCATCCTGGTCGAGCTGGACGGCGACGTAAACGGCCACAAGTTCAGCGTGTCCGGCGAGGGCGAGGGCGATGCCACCTACGGCAAGCTGACCCTGAAGTTCATCTGCACCACCGGCAAGCTGCCCGTGCCCTGGCCCACCCTCGTGACCACCCTGACCTACGGCGTGCAGTGCTTCAGCCGCTACCCCGACCACATGAAGCAGCACGACTTCTTCAAGTCCGCCATGCCCGAAGGCTACGTCCAGGAGCGCACCATCTTCTTCAAGGACGACGGCAACTACAAGACCCGCGCCGAGGTGAAGTTCGAGGGCGACACCCTGGTGAACCGCATCGAGCTGAAGGGCATCGACTTCAAGGAGGACGGCAACATCCTGGGGCACAAGCTGGAGTACAACTACAACAGCCACAACGTCTATATCATGGCCGACAAGCAGAAGAACGGCATCAAGGTGAACTTCAAGATCCGCCACAACATCGAGGACGGCAGCGTGCAGCTCGCCGACCACTACCAGCAGAACACCCCCATCGGCGACGGCCCCGTGCTGCTGCCCGACAACCACTACCTGAGCACCCAGTCCGCCCTGAGCAAAGACCCCAACGAGAAGCGCGATCACATGGTCCTGCTGGAGTTCGTGACCGCCGCCGGGATCACTCTCGGCATGGACGAGCTGTACAAGTAAAGCGGCCGCGACATGACATCGATACCGTCGACCTCGATCGAGACCTAGAAAAACATGGAGCAATCACAAGTAGCAATACAGCAGCTACCAATGCTGATTGTGCCTGGCTAGAAGCACAAGAGGAGGAGGAGGTGGGTTTTCCAGTCACACCTCAGGTACCTTTAAGACCAATGACTTACAAGGCAGCTGTAGATCTTAGCCACTTTTTAAAAGAAAAGGGGGGACTGGAAGGGCTAATTCACTCCCAACGAAGACAAGATATCCTTGATCTGTGGATCTACCACACACAAGGCTACTTCCCTGATTGGCAGAACTACACACCAGGGCCAGGGATCAGATATCCACTGACCTTTGGATGGTGCTACAAGCTAGTACCAGTTGAGCAAGAGAAGGTAGAAGAAGCCAATGAAGGAGAGAACACCCGCTTGTTACACCCTGTGAGCCTGCATGGGATGGATGACCCGGAGAGAGAAGTATTAGAGTGGAGGTTTGACAGCCGCCTAGCATTTCATCACATGGCCCGAGAGCTGCATCCGGACTGTACTGGGTCTCTCTGGTTAGACCAGATCTGAGCCTGGGAGCTCTCTGGCTAACTAGGGAACCCACTGCTTAAGCCTCAATAAAGCTTGCCTTGAGTGCTTCAAGTAGTGTGTGCCCGTCTGTTGTGTGACTCTGGTAACTAGAGATCCCTCAGACCCTTTTAGTCAGTGTGGAAAATCTCTAGCAGCATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAATCTAAAGTATATATGAGTAAACTTGGTCTGACAGTTACCAATGCTTAATCAGTGAGGCACCTATCTCAGCGATCTGTCTATTTCGTTCATCCATAGTTGCCTGACTCCCCGTCGTGTAGATAACTACGATACGGGAGGGCTTACCATCTGGCCCCAGTGCTGCAATGATACCGCGAGACCCACGCTCACCGGCTCCAGATTTATCAGCAATAAACCAGCCAGCCGGAAGGGCCGAGCGCAGAAGTGGTCCTGCAACTTTATCCGCCTCCATCCAGTCTATTAATTGTTGCCGGGAAGCTAGAGTAAGTAGTTCGCCAGTTAATAGTTTGCGCAACGTTGTTGCCATTGCTACAGGCATCGTGGTGTCACGCTCGTCGTTTGGTATGGCTTCATTCAGCTCCGGTTCCCAACGATCAAGGCGAGTTACATGATCCCCCATGTTGTGCAAAAAAGCGGTTAGCTCCTTCGGTCCTCCGATCGTTGTCAGAAGTAAGTTGGCCGCAGTGTTATCACTCATGGTTATGGCAGCACTGCATAATTCTCTTACTGTCATGCCATCCGTAAGATGCTTTTCTGTGACTGGTGAGTACTCAACCAAGTCATTCTGAGAATAGTGTATGCGGCGACCGAGTTGCTCTTGCCCGGC